(19) United States
(12) Patent Application Publication
(10) Pub. No.: US 2006/0014212 A1
(43) Pub. Date: Jan. 19, 2006

(54) Proteome epitope tags and methods of use thereof in protein modification analysis

(75) Inventors: Stephen J. Benkovic, State College, PA (US); John W. Chan, Research Triangle Park, NC (US); Frank D. Lee, Chestnut Hill, MA (US); Xun Meng, Newton, MA (US); Neal Gordon, Lexington, MA (US)

Correspondence Address: FISH & NEAVE IP GROUP;ROPES & GRAY LLP, ONE INTERNATIONAL PLACE, BOSTON, MA, 02110-2624, US

(73) Assignee: Epitome Biosystems, Inc., Waltham, MA (US)

(21) Appl. No.: 11/066,967

(22) Filed: Feb. 25, 2005

Related U.S. Application Data
(63) Continuation-in-part of application No. 10/773,032, filed on Feb. 5, 2004.
Application No. 10/773,032 is a continuation-in-part of application No. 10/712,425, filed on Nov. 13, 2003.
Application No. 10/712,425 is a continuation-in-part of application No. 10/436,549, filed on May 12, 2003.
(60) Provisional application No. 60/379,626, filed on May 10, 2002.
(60) Provisional application No. 60/393,197, filed on Jul. 1, 2002.
(60) Provisional application No. 60/393,233, filed on Jul. 1, 2002.
(60) Provisional application No. 60/393,235, filed on Jul. 1, 2002.
(60) Provisional application No. 60/393,211, filed on Jul. 1, 2002.
(60) Provisional application No. 60/393,223, filed on Jul. 1, 2002.
(60) Provisional application No. 60/393,280, filed on Jul. 1, 2002.
(60) Provisional application No. 60/393,137, filed on Jul. 1, 2002.
(60) Provisional application No. 60/430,948, filed on Dec. 4, 2002.
(60) Provisional application No. 60/433,319, filed on Dec. 13, 2002.

(51) Int. Cl. G01N 33/53; G06F 19/00

(52) U.S. Cl. 435/7.1; 702/19

(57) ABSTRACT

Disclosed are reagents and methods for reliably detecting the presence and measuring the amount of proteins, including proteins with various post-translational modifications (phosphorylation, glycosylation, methylation, acetylation, etc.) in a sample by the use of one or more capture agents that recognize and interact with recognition sequences uniquely characteristic of a protein or a set of proteins (Proteome Epitope Tags, or PETs) in the sample. Arrays comprising these capture agents or PETs are also provided.

### CROSS-REFERENCE TO RELATED APPLICATIONS

This application is a continuation-in-part application of U.S. Ser. No. 10/773,032, filed on Feb. 5, 2004, which is a continuation-in-part application of U.S. Ser. No. 10/712,425, filed on Nov. 13, 2003, which is a continuation-in-part application of U.S. Ser. No. 10/436,549, filed on May 12, 2003, which claims priority to U.S. Provisional Application No. 60/379,626, filed on May 10, 2002; U.S. Provisional Application Nos. 60/393,137, 60/393,233, 60/393,235, 60/393,211, 60/393,223, 60/393,280, and 60/393,197, all filed on Jul. 1, 2002; U.S. Provisional Application No. 60/430,948, filed on Dec. 4, 2002; and U.S. Provisional Application No. 60/433,319 filed on Dec. 13, 2002, the entire contents of each of which are incorporated herein by reference.

### BACKGROUND OF THE INVENTION

Genomic studies are now approaching “industrial” speed and scale, thanks to advances in gene sequencing and the increasing availability of high-throughput methods for studying genes, the proteins they encode, and the pathways in which they are involved. The development of DNA microarrays has enabled massively parallel studies of gene expression as well as genomic DNA variations.

DNA microarrays have shown promise in advanced medical diagnostics. More specifically, several groups have shown that when the gene expression patterns of normal and diseased tissues are compared at the whole genome level, patterns of expression characteristic of the particular disease state can be observed. Bittner et al., (2000) Nature 406:536-540; Clark et al., (2000) Nature 406:532-535; Huang et al., (2001) Science 294:870-875; and Hughes et al., (2000) Cell 102:109-126. For example, tissue samples from patients with malignant forms of prostate cancer display a recognizably different pattern of mRNA expression to tissue samples from patients with a milder form of the disease. C.f., Dhanasekaran et al., (2001) Nature 412 (2001), pp. 822-826.

However, as James Watson pointed out recently proteins are really the “actors' in biology” (“A Cast of Thousands” Nature Biotechnology March 2003). A more attractive approach would be to monitor key proteins directly. These might be biomarkers identified by DNA microarray analysis. In this case, the assay required might be relatively simple, examining only 5-10 proteins. Another approach would be to use an assay that detects hundreds or thousands of protein features, such as for the direct analysis of blood, sputum or urine samples, etc. It is reasonable to believe that the body would react in a specific way to a particular disease state and produce a distinct “biosignature” in a complex data set, such as the levels of 500 proteins in the blood. One could imagine that in the future a single blood test could be used to diagnose most conditions.

The motivation for the development of large-scale protein detection assays as basic research tools is different to that for their development for medical diagnostics. The utility of biosignatures is one aspect researchers desire in order to understand the molecular basis of cellular response to a particular genetic, physiological or environmental stimulus. DNA microarrays do a good job in this role, but detection of proteins would allow for more accurate determination of protein levels and, more importantly, could be designed to quantitate the presence of different splice variants or isoforms. These events, to which DNA microarrays are largely or completely blind, often have pronounced effects on protein activities.

This has sparked great interest in the development of devices such as protein-detecting microarrays (PDMs) to allow similar experiments to be done at the protein level, particularly in the development of devices capable of monitoring the levels of hundreds or thousands of proteins simultaneously.

Prior to the present invention, PDMs that even approach the complexity of DNA microarrays do not exist. There are several problems with the current approaches to massively parallel, e.g., cell-wide or proteome wide, protein detection. First, reagent generation is difficult: One needs to first isolate every individual target protein in order to isolate a detection agent against every protein in an organism and then develop detection agents against the purified protein. Since the number of proteins in the human organism is currently estimated to be about 30,000 this requires a lot of time (years) and resources. Furthermore, detection agents against native proteins have less defined specificity since it is a difficult task to know which part of the proteins the detection agents recognize. This problem causes considerable cross-reactivity of when multiple detection agents are arrayed together, making large-scale protein detection array difficult to construct. Second, current methods achieve poor coverage of all possible proteins in an organism. These methods typically include only the soluble proteins in biological samples. They often fail to distinguish splice variants, which are now appreciated as being ubiquitous. They exclude a large number of proteins that are bound in organellar and cellular membranes or are insoluble when the sample is processed for detection. Third, current methods are not general to all proteins or to all types of biological samples. Proteins vary quite widely in their chemical character. Groups of proteins require different processing conditions in order to keep them stably solubilized for detection. Any one condition may not suit all the proteins. Further, biological samples vary in their chemical character. Individual cells considered identical express different proteins over the course of their generation and ultimate death. Physiological fluids like urine and blood serum are relatively simple, but biopsy tissue samples are very complex. Different protocols need to be used to process each type of sample and achieve maximal solubilization and stabilization of proteins.

Current detection methods are either not effective over all proteins uniformly or cannot be highly multiplexed to enable simultaneous detection of a large number of proteins (e.g., >5,000). Optical detection methods would be most cost effective but suffer from lack of uniformity over different proteins. Proteins in a sample have to be labeled with dye molecules and the different chemical character of proteins leads to inconsistency in efficiency of labeling. Labels may also interfere with the interactions between the detection agents and the analyte protein leading to further errors in quantitation. Non-optical detection methods have been developed but are quite expensive in instrumentation and are very difficult to multiplex for parallel detection of even moderately large samples (e.g., >100 samples).

Another problem with current technologies is that they are burdened by intracellular life processes involving a complex web of protein complex formation, multiple enzymatic reactions altering protein structure, and protein conformational changes. These processes can mask or expose binding sites known to be present in a sample. For example, prostate specific antigen (PSA) is known to exist in serum in multiple forms including free (unbound) forms, e.g., pro-PSA, BPSA (BPH-associated free PSA), and complexed forms, e.g., PSA-ACT, PSA-A2M (PSA-alpha₂-macroglobulin), and PSA-API (PSA-alpha₁-protease inhibitor) (see Stephan C. et al. (2002) Urology 59:2-8). Similarly, Cyclin E is known to exist not only as a full length 50 kD protein, but also in five other low molecular weight forms ranging in size from 34 to 49 kD. In fact, the low molecular weight forms of cyclin E are believed to be more sensitive markers for breast cancer than the full length protein (see Keyomarsi K. et al. (2002) N. Eng. J. Med. 347(20):1566-1575).

Sample collection and handling prior to a detection assay may also affect the nature of proteins that are present in a sample and, thus, the ability to detect these proteins. As indicated by Evans M. J. et al. (2001) Clinical Biochemistry 34:107-112 and Zhang D. J. et al. (1998) Clinical Chemistry 44(6):1325-1333, standardizing immunoassays is difficult due to the variability in sample handling and protein stability in plasma or serum. For example, PSA sample handling, such as sample freezing, affects the stability and the relative levels of the different forms of PSA in the sample (Leinonen J, Stenman U H (2000) Tumour Biol. 21(1):46-53).

Finally, current technologies are burdened by the presence of autoantibodies which affect the outcome of immunoassays in unpredictable ways, e.g., by leading to analytical errors (Fitzmaurice T. F. et al. (1998) Clinical Chemistry 44(10):2212-2214).

These problems prompted the question whether it is even possible to standardize immunoassays for hetergenous protein antigens. (Stenman U-H. (2001) Immunoassay Standardization: Is it possible? Who is responsible? Who is capable? Clinical Chemistry 47 (5) 815-820). Thus, a great need exists in the art for efficient and simple methods of parallel detection of proteins that are expressed in a biological sample and, particularly, for methods that can overcome the imprecisions caused by the complexity of protein chemistry and for methods which can detect all or a majority of the proteins expressed in a given cell type at a given time, or for proteome-wide detection and quantitation of proteins expressed in biological samples.

### SUMMARY OF THE INVENTION

The present invention is directed to methods and reagents for reproducible protein detection and quantitation, e.g., parallel detection and quantitation, in complex biological samples. Salient features to certain embodiments of the present invention reduce the complexity of reagent generation, achieve greater coverage of all protein classes in an organism, greatly simplify the sample processing and analyte stabilization process, and enable effective and reliable parallel detection, e.g., by optical or other automated detection methods, and quantitation of proteins and/or post-translationally modified forms, and, enable multiplexing of standardized capture agents for proteins with minimal cross-reactivity and well-defined specificity for large-scale, proteome-wide protein detection.

Embodiments of the present invention also overcome the imprecisions in detection methods caused by: the existence of proteins in multiple forms in a sample (e.g., various post-translationally modified forms or various complexed or aggregated forms); the variability in sample handling and protein stability in a sample, such as plasma or serum; and the presence of autoantibodies in samples. In certain embodiments, using a targeted fragmentation protocol, the methods of the present invention assure that a binding site on a protein of interest, which may have been masked due to one of the foregoing reasons, is made available to interact with a capture agent. In other embodiments, the sample proteins are subjected to conditions in which they are denatured, and optionally are alkylated, so as to render buried (or otherwise cryptic) PET moieties accessible to solvent and interaction with capture agents. As a result, the present invention allows for detection methods having increased sensitivity and more accurate protein quantitation capabilities. This advantage of the present invention will be particularly useful in, for example, protein marker-type disease detection assays (e.g., PSA or Cyclin E based assays) as it will allow for an improvement in the predictive value, sensitivity, and reproducibility of these assays. The present invention can standardize detection and measurement assays for all proteins from all samples.

For example, a recent study by Punglia et al. (N. Engl. J. Med. 349(4): 335-42, July, 2003) indicated that, in the standard PSA-based screening for prostate cancer, if the threshold PSA value for undergoing biopsy were set at 4.1 ng per milliliter, 82 percent of cancers in younger men and 65 percent of cancers in older men would be missed. Thus a lower threshold level of PSA for recommending prostate biopsy, particularly in younger men, may improve the clinical value of the PSA test. However, at lower detection limits, background can become a significant issue. It would be immensly advantageous if the sensitivity/selectivity of the assay can be improved by, for example, the method of the instant invention.

In a specific embodiment, the invention provides a method to detect and quantitate the presence of specific modified polypeptides in a sample. In a general sense, the invention provides a method to identify a URS or PET uniquely associated with a modification site on a peptide fragment, which PET can then be captured and detected/quantitated by specific capture agents. The method applies to virtually all kinds of post-translational modifications, including but are not limited to phosphorylation, glycosylation, etc., as long as the modification can be reliably detected, for example, by phospho-antibodies. The method also applies to the detection of alternative splicing forms of otherwise identical proteins.

The present invention is based, at least in part, on the realization that exploitation of unique recognition sequences (URSs) or Proteome Epitope Tags (PETs) present within individual proteins can enable reproducible detection and quantitation of individual proteins in parallel in a milieu of proteins in a biological sample. As a result of this PET-based approach, the methods of the invention detect specific proteins in a manner that does not require preservation of the whole protein, nor even its native tertiary structure, for analysis. Moreover, the methods of the invention are suitable for the detection of most or all proteins in a sample, including insoluble proteins such as cell membrane bound and organelle membrane bound proteins.

The present invention is also based, at least in part, on the realization that PETs can serve as Proteome Epitope Tags characteristic of a specific organism's proteome and can enable the recognition and detection of a specific organism.

The present invention is also based, at least in part, on the realization that high-affinity agents (such as antibodies) with predefined specificity can be generated for defined, short length peptides and when antibodies recognize protein or peptide epitopes, only 4-6 (on average) amino acids are critical. See, for example, Lerner R A (1984) Advances In Immunology. 36:1-45.

The present invention is also based, at least in part, on the realization that by denaturing (including thermo- and/or chemical-denaturation) and/or fragmenting (such as by protease digestion including digestion by thermo-protease) all proteins in a sample to produce a soluble set of protein analytes, e.g., in which even otherwise buried PETs including PETs in protein complexes/aggregates are solvent accessible, the subject method provides a reproducible and accurate (intra-assay and inter-assay) measurement of proteins.

The present invention is also based, at least in part, on the realization that protein modifications associated with PETs on a fragmented peptide can be readily detected and quantitated by isolating the associated PET followed by detection/quantitation of the modification.

Accordingly, in one aspect, the present invention provides a method for globally detecting the presence of a protein(s) (e.g., membrane bound protein(s)) in an organism's proteome. The method includes providing a sample which has been denatured and/or fragmented to generate a collection of soluble polypeptide analytes; contacting the polypeptide analytes with a plurality of capture agents (e.g., capture agents immobilized on a solid support such as an array) under conditions such that interaction of the capture agents with corresponding unique recognition sequences occurs, thereby globally detecting the presence of protein(s) in an organism's proteome.

The method is suitable for use in, for example, diagnosis (e.g., clinical diagnosis or environmental diagnosis), drug discovery, protein sequencing or protein profiling. In one embodiment, at least 50%, 55%, 60%, 65%, 70%, 75%, 80%, 85%, 90%, 95% or 100% of an organism's proteome is detectable from arrayed capture agents.

The capture agent may be a protein, a peptide, an antibody, e.g., a single chain antibody, an artificial protein, an RNA or DNA aptamer, an allosteric ribozyme, a small molecule or electronic means of capturing a PET.

The sample to be tested (e.g., a human, yeast, mouse, C. elegans, Drosophila melanogaster or Arabidopsis thaliana sample, such whole cell lysate) may be fragmented by the use of a proteolytic agent. The proteolytic agent can be any agent, which is capable of predictably cleaving polypeptides between specific amino acid residues (i.e., the proteolytic cleavage pattern). The predictability of cleavage allows a computer to generate fragmentation patterns in sillico, which will greatly aid the process of searching PETs unique to a sample.

According to one embodiment of this aspect of the present invention a proteolytic agent is a proteolytic enzyme. Examples of proteolytic enzymes, include but are not limited to trypsin, calpain, carboxypeptidase, chymotrypsin, V8 protease, pepsin, papain, subtilisin, thrombin, elastase, gluc-C, endo lys-C or proteinase K, caspase-1, caspase-2, caspase-3, caspase-4, caspase-5, caspase-6, caspase-7, caspase-8, MetAP-2, adenovirus protease, HIV protease and the like.

The following table summarizes the result of analyzing pentamer PETs in the human proteome using different proteases. A total of 23,446 sequences are tagged before protease digestion.

| | | Fragment | Tagged |
|---|---|---|---|
| Protease | Cleavage Site | Length | Proteins |
| Chymotrypsin | after W,F,Y | 12.7 | 21,990 |
| S.A. V-8 E specific | after E | 13.7 | 23,120 |
| Post-Proline Cleaving Enzyme | after P | 15.7 | 23,009 |
| Trypsin | after K, R | 8.5 | 22,408 |

According to another embodiment of this aspect of the present invention a proteolytic agent is a proteolytic chemical such as cyanogen bromide and 2-nitro-5-thiocyanobenzoate. In still other embodiments, the proteins of the test sample can be fragmented by physical shearing; by sonication, or some combination of these or other treatment steps.

An important feature for certain embodiments, particularly when analyzing complex samples, is to develop a fragmentation protocol that is known to reproducibly generate peptides, preferably soluble peptides, which serve as the unique recognition sequences. The collection of polypeptide analytes generated from the fragmentation may be 5-30, 5-20, 5-10, 10-20, 20-30, or 10-30 amino acids long, or longer. Ranges intermediate to the above recited values, e.g., 7-15 or 15-25 are also intended to be part of this invention. For example, ranges using a combination of any of the above recited values as upper and/or lower limits are intended to be included.

The unique recognition sequence may be a linear sequence or a non-contiguous sequence and may be 5, 6, 7, 8, 9, 10, 11, 12, 13, 14, 15, 16, 17, 18, 19, 20, 25, or 30 amino acids in length. In certain embodiments, the unique recognition sequence is selected from the group consisting of all or a sub-collection of the SEQ ID Nos disclosed herein.

In another aspect, the present invention provides a method for detecting the presence of a protein, preferably simultaneous or parallel detection of multiple proteins, in a sample. The method includes providing a sample which has been denatured and/or fragmented to generate a collection of soluble polypeptide analytes; providing an array comprising a support having a plurality of discrete regions to which are bound a plurality of capture agents, wherein each of the capture agents is bound to a different discrete region and wherein each of the capture agents is able to recognize and interact with a unique recognition sequence Within a protein; contacting the array of capture agents with the polypeptide analytes; and determining which discrete regions show specific binding to the sample, thereby detecting the presence of a protein in a sample.

Thus one aspect of the invention provides a method for obtaining one or more capture agent(s) for identifying one or more target proteins in a sample, the method comprising: (1) computationally identifying the amino acid sequences of one or more fragments of each the target proteins expected to be present in a variegated sample of proteins, the fragments predictably resulting from a treatment of the target proteins, and each of the fragments encompassing one or more unique PET (proteome epitope tag) sequences; (2) generating reference reagents for each the unique PET sequences; (3) obtaining a set of capture agents, each of which selectively binds a PET sequence of one of the reference reagents, wherein collectively the set of capture agents can bind and identify the occurrence of the target proteins present in the sample under conditions wherein the capture agents are contacted with the target proteins, or the fragments thereof, that have been rendered soluble in solution.

In one embodiment, the step of computationally identifying amino acid sequences may include a Nearest-Neighbor Analysis that identifies PET sequences based on criteria that may include one or more of pI, charge, steric, solubility, hydrophobicity, polarity and solvent exposed area.

In one embodiment, the PET sequence is about 5-30 amino acids in length, preferably about 5-10 amino acids in length, most preferably about 8 amino acids.

In one embodiment, the capture agents may be full-length antibodies, or functional antibody fragments, such as Fab fragments, F(ab′)2 fragments, Fd fragments, Fv fragments, dAb fragments, isolated complementarity determining regions (CDR), single chain antibodies (scFv), or derivatives thereof.

In one embodiment, at least about 50%, 60%, 70%, 80%, 90% or more of all of the antibodies or functional antibody fragments have affinity constants of no more than about 10 nM.

In one embodiment, the method further comprises determining the specificity of the antibodies or functional antibody fragments against one or more nearest neighbor antigens, if any, of the PETs, and selecting antibodies or functional antibody fragments that do not substantially cross-react with any other antigens, including their nearest neighbor antigens.

As used herein, “do not substantially cross-react” means that the PET anibodies are specific for the antigen PET sequences against which they are raised, respectively. These PET antobodies have affinity for the respective PET antigens that are at least about 10, 20, 50, 100, 200, 500, 1000-fold or higher as compared to those for any other antigens, including their respective nearest neighbors.

In one embodiment, the method further comprises derivatizing the capture agents with a detectable label, such as a fluorescent label, a stainable dye, a chemilumninescent compound, a colloidal particle, a radioactive isotope, a near-infrared dye, a DNA dendrimer, a water-soluble quantum dot, a latex bead, a selenium particle, or a europium nanoparticle.

In one embodiment, the reference reagents are natural or synthesized antigens comprising the PET sequence, and wherein the N- or C-terminus, or both, of the PET sequence (or reference reagents comprising the PET sequence) are blocked to eliminate free N- or C-terminus, or both. These reference reagents may be purified natural peptides, or their synthesized counterparts, or synthetic peptides of any desired sequences.

In one embodiment, step (3) is effectuated by screening libraries of antibodies or functional antibody fragments, or by de novo antibody production and screening using immunized animals.

Another aspect of the invention relates to a method for simultaneously detecting and/or measuring a plurality of target proteins in a sample, the method comprising: (1) using the method of the instant invention described above, obtaining a plurality of capture agents, each specific for one PET sequence of one of the target proteins, wherein each of the plurality of target proteins is recognized by at least one of the plurality of capture agents; (2) treating the sample with a predetermined protocol to generate a plurality of poplypeptide fragments, wherein for each of the target proteins, at least one of its polypeptide fragments comprises at least one PET sequence recognized by at least one of the capture agents; (3) contacting at least a portion of the treated sample with the plurality of capture agents, and, (4) detecting/measuring binding events, thereby simultaneously detecting and/or measuring the plurality of target proteins in the sample.

In one embodiment, the predetermined protocol comprises denaturing and/or proteolysis of the sample.

In one embodiment, the sample may be one or more (e.g. mixture) of: saliva, mucous, sweat, whole blood, plasma, serum, urine, amniotic fluid, genital fluid, fecal material, marrow, plasma, spinal fluid, pericardial fluid, gastric fluid, abdominal fluid, peritoneal fluid, pleural fluid, synovial fluid, cyst fluid, cerebrospinal fluid, lung lavage fluid, lymphatic fluid, tears, prostatitc fluid, extraction from other body parts, secretion from other glands, supernatant, whole cell lysate, cell fraction obtained by lysis and fractionation of cellular material, extract or fraction of cells obtained directly from a biological entity or cells grown in an artificial environment.

In one embodiment, the proteolysis is effected by a protease, such as trypsin, chymotrypsin, pepsin, papain, carboxypeptidase, calpain, subtilisin, gluc-C, endo lys-C or proteinase K.

In one embodiment, the denaturing is effected by thermo-denaturation or chemical denaturation.

In one embodiment, the thermo-denaturation is followed by or concurrent with proteolysis using thermo-stable proteases.

In one embodiment, each of the capture agents is immobilized on a solid support at an addressable location; and wherein for each of the target proteins, each of the at least one of its polypeptide fragments further comprises a second PET sequence or a post-translational modification site.

In one embodiment, the post-translational modification site is a phosphorylation site for phospho-Tyr, Phospho-Ser, or phospho-Thr.

In one embodiment, the phosphorylation site is phosphorylated, and wherein step (4) is effectuated by detecting/measuring the second PET and/or phospho-amino acid at the phosphorylation site by a detectable agent (e.g. labeled secondary antibody or a fluorescent dye).

In one embodiment, each of the capture agents is immobilized on a solid support at an addressable location; and wherein step (3) further comprises simultaneously contacting the capture agents with labeled standard competition peptides, the labeled standard competition peptides are detected/measured in (4).

In one embodiment, each of the capture agents is immobilized on a solid support at an addressable location; wherein step (2) further comprises labeling the plurality of polypeptide fragments with a first label; wherein step (3) further comprises simultaneously contacting the capture agents with standard competition peptides labeled by a second label, the first and second labels are detected/measured in (4).

In one embodiment, each of the capture agents is immobilized on a solid support at an addressable location; and wherein at least one of the target proteins is represented by at least two polypeptide fragments, each comprising at least one PET sequence recognized by at least one of the capture agents.

In one embodiment, the method further comprises generating an array of reference peptide fragments, each immobilized on an addressable location on a solid support, wherein each of the reference peptide fragments corresponds to one of the at least one polypeptide fragments; and wherein step (3) is carried out on the array.

In one embodiment, step (4) is effectuated by detecting/measuring the capture agents bound to the arrays.

All claims as recited in the summary sections in the parent applications, U.S. Ser. No. 10/773,032 (filed on Feb. 5, 2004), U.S. Ser. No. 10/712,425 (filed on Nov. 13, 2003), and U.S. Ser. No. 10/436,549 (filed on May 12, 2003), are incorporated herein by reference. It is also contemplated that all embodiments of the invention, including those specifically described herein and in the parent applications for different aspects of the invention, can be combined with any other embodiments of the invention as appropriate.

Other features and advantages of the invention will be apparent from the following detailed description and claims.

### BRIEF DESCRIPTION OF THE DRAWINGS

FIG. 1 is an alternative format for the parallel detection of PET from a complex sample. In this type of “virtual array” each of many different beads displays a capture agent directed against a different PET. Each different bead is color-coded by covalent linkage of two dyes (dye1 and dye2) at a characteristic ratio. Only two different beads are shown for clarity. Upon application of the sample, the capture agent binds a cognate PET, if present in the sample. Then a mixture of secondary binding ligands (in this case labeled PET peptides) conjugated to a third fluorescent tag is applied to the mixture of beads. The beads can then be analyzed using flow cytometry other detection method that can resolve, on a bead-by-bead basis, the ratio of dye1 and dye2 and thus identify the PET captured on the bead, while the fluorescence intensity of dye3 is read to quantitate the amount of labeled PET on the bead (which will in inversely reflect the analyte PET level).

FIG. 2 illustrates the result of extraction of intracellular and membrane proteins. Top Panel: M: Protein Size Marker; H-S: HELA-Supernatant; H-P: HELA-Pellet; M-S: MOLT4-Supernatant; M-P: MOLT4-Pellet. Bottom panel shows that >90% of the proteins are solublized. Briefly, cells were washed in PBS, then suspended (5×10⁶ cells/ml) in a buffer with 0.5% Triton X-100 and homogenized in a Dounce homogenizer (30 strokes). The homogenized cells were centrifuged to separate the soluble portion and the pellet, which were both loaded to the gel.

FIG. 3 illustrates the process for PET-specific antibody generation.

FIG. 4 illustrates a general scheme of sample preparation prior to its use in the methods of the instant invention. The left side shows the process for chemical denaturation followed by protease digestion, the right side illustrates the preferred thermo-denaturation and fragmentation. Although the most commonly used protease trypsin is depicted in this illustration, any other suitable proteases described in the instant application may be used. The process is simple, robust & reproducible, and is generally applicable to main sample types including serum, cell lysates and tissues.

FIG. 5 provides an illustrative example of serum sample pre-treatment using either the thermo-denaturation or the chemical denaturation as described in FIG. 4.

FIG. 6 shows the result of thermo-denaturation and chemical denaturation of serum proteins and cell lysates (MOLT4 and Hela cells).

FIG. 7 illustrates the structure of mature TGF-beta dimer, and one complex form of mature TGF-beta with LAP and LTBP.

FIG. 8 depicts PET-based array for (AKT) kinase substrate identification.

FIG. 9 illustrates a schematic drawing of fluorescence sandwich immunoassay for specific capture and quantitation of a targeted peptide in a complex peptide mixture, and results of readout fluorescent signal detected by the secondary antibody.

FIG. 10 illustrates the sandwich assay used to detect a tagged-human PSA protein.

FIG. 11 illustrates the PETs and their nearest neighbors for the detection of phospho-peptides in SHIP-2 and ABL.

FIG. 12 illustrates a general approach to use the sandwich assay for detecting N proteins with N+1 PET-specific antibodies.

FIG. 13 illustrates the common PETs and kinase-specific PETs useful for the detection of related kinases.

FIG. 14 shows a design for the PET-based assay for standardized serum TGF-beta measurement.

FIG. 15 is a schematic drawing showing the general principal of detecting PET-associated protein modification using sandwich assay.

FIG. 16 is a schematic diagram of one embodiment of the detection of post-translational modification (e.g., phosphorylation or glycosylation). A target peptide is digested by a protease, such as Trypsin to yield smaller, PET-containing fragments. One of the fragments (PTP2) also contains at least one modification of interest. Once the fragments are isolated by capture agents on a support, the presence of phosphorylation can be detected by, for example, HRP-conjugated anti-phospho-amino acid antibodies; and the presence of sugar modification can be detected by, for example, lectin.

FIG. 17 illustrates that PET-specific antibodies are highly specific for the PET antigen and do not bind the nearest neighbors of the PET antigen.

FIG. 18 shows PETs for ERK1/2 and MEK1/2. Two tryptic fragments are measured: the non-phoshorylated peptide for total protein concentration and the phosphopeptide for activated protein state measurement. The predicted tryptic peptide sequences are displayed with the PET sequences shown in either Blue or Red (or underlined). The phosphorylated residues of the activated proteins are shown as circles with Ps.

FIG. 19 is a Western blot analysis of cell extracts with an anti-PET antibody. Expected size (42 kDa) of ERK1/2 proteins is detected using an anti-A008 antibody.

FIG. 20 shows that anti-PET antibodies do not cross-react with selected nearest neighbor peptides. Anti-A007 and A008 antibodies are shown specifically to recognize the native A007 and A008 PET-containing peptides (blue colors) but show insignificant binding towards nearest neighbor peptides (red colors). The X axis shows the peptide sequences and Y axis is the fluorescence signal measured using fluorescently labeled antibodies binding to peptides on an array.

FIG. 21 shows PET peptide sandwich assays on an antibody chip. An antibody array with 16 individual reaction chambers is shown. Synthetic peptides (total and phosphorylated tryptic peptides) are used as quantification standards for developing the standard curve. A typical fluorescence image for sandwich assay is shown with increasing concentration of a peptide binding to the printed antibodies.

FIG. 22 shows Trypsin digestion of total protein extract from human cells. Protein extract from Jurkat cells was loaded at 20 μg (100%), 10 μg (50%), 4 μg (20%) and 1 μg (5%). The trypsin digested protein was loaded at 20 μg per lane (100% Digested). Since intact proteins on the 100% Digested lane are less than that from 5% lane, it is estimated that >95% of the proteins are digested by trypsin.

FIG. 23 A) LC/MS/MS data on trypsin digested MEK1 protein. Two expected tryptic peptides are detected as shown. B) The use of a sandwich assay on the PET chip to monitor the trypsin digestion of MEK1 protein. The fluorescence signal increases as the concentration of the digested MEK1 increases. The lowest detection limit is between 0 and 75 pM, consistent with data presented in Table A4.

FIG. 24 Western analysis of the activation of Ras pathway. Total MEK1/2 and ERK1/2 proteins from Jurkat cells are probed using anti-A011 and anti-A007 antibodies (Total Protein) and monoclonal antibodies for pMEK1/2 and pERK1/2. Unstimulated Cells are labeled as − and stimulated by PMA as +.

FIG. 25 shows fluorescence images of sandwich assays on PET chips for generating data in Table A5.

FIG. 26 shows specificity of PET-antibodies (A) on a 33-plex peptide array (each peptide is printed in 5 replicates, and the whole array is probed individually with one PET-antibody at a time), and (B) using a competitive assay format against top four abaundant serum proteins.

FIG. 27 is a schematic drawing illiustrating the general approach of the peptide array competition assay.

FIG. 28 shows exemplary standard competition curves for two of the arrayed PET peptides A024 (IL1-β) and A014 (Thyroglobulin).

FIG. 29 is a schematic drawing illiustrating the general approach of the antibody array competition assay.

FIG. 30 shows exemplary standard competition curves for two of the arrayed PET antibodies against A034 (C4) and A047 (Fibronectin), respectively.

FIG. 31 is a schematic drawing illiustrating the general approach of the ratio format antibody array competition assay.

FIG. 32 shows an exemplary result of ratio format antibody competition assay for TGF-β1.

FIG. 33 is a schematic drawing illiustrating the general approach of antibody sandwich assay.

FIG. 34 shows an exemplary result of sandwich assay for PSA detection. Two PET antibodies were used to detect a PSA tryptic fragment.

FIG. 35 shows SDS PAGE analysis of tryptic digestion for both human serum and E. coli lysate, stained with SyproRuby and imaged by fluorescence. A dilution series of intact sample is compared with a much higher concentration of the digest in order to quantify the amount if intact protein remaining in the digest. In all case the digest contained less intact protein than the 5% sample.

FIG. 36 shows precipitation upon denaturation/reduction due to high protein concentration: the “Fried Egg” effect. Human serum reduced and denatured at 100° C. after dilution in 200 mM NaHCO₃. At least a 1:2 dilution is required=20 mg/ml protein in serum.

FIG. 37 shows the rapid digestion time course. Serum (100 μl) was reduced and alkylated (diluted to 460 μl) and then digested with trypsin at 37° C. (30 μl Trypsin at 10 mg/ml). Removed aliquot for T0 prior to addition of trypsin, immediately removed aliquot for 0.1 min time point and then placed at 37° C. removing aliquots at the time points indicated. Removed additional aliquots at 1, 2, 5, and 10 min and a final aliquot after over night digestion at 37° C.

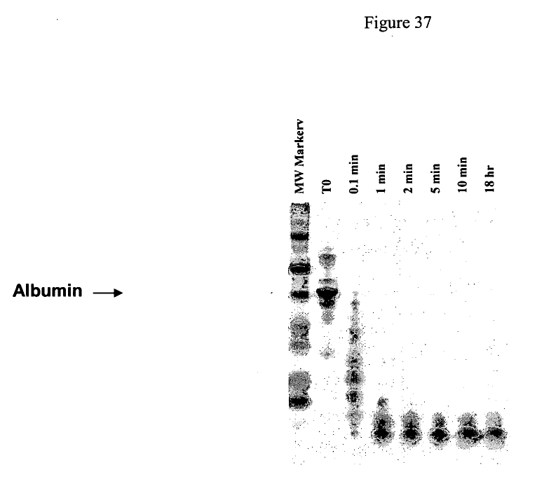

FIG. 38 shows an example of redundant protein measurement for Fibronectin.

### DETAILED DESCRIPTION OF THE INVENTION

The present invention provides methods, reagents and systems for detecting, e.g., globally detecting, the presence of a protein or a panel of proteins, especially protein with a specific type of modification (phosphorylation, glycosylation, alternative splicing, mutation, etc.) in a sample. In certain embodiments, the method may be used to quantitate the level of expression or post-translational modification of one or more proteins in the sample. The method includes providing a sample which has, preferably, been fragmented and/or denatured to generate a collection of peptides, and contacting the sample with a plurality of capture agents, wherein each of the capture agents is able to recognize and interact with a unique recognition sequence (URS) or PET characteristic of a specific protein or modified state. Through detection and deconvolution of binding data, the presence and/or amount of a protein in the sample is determined.

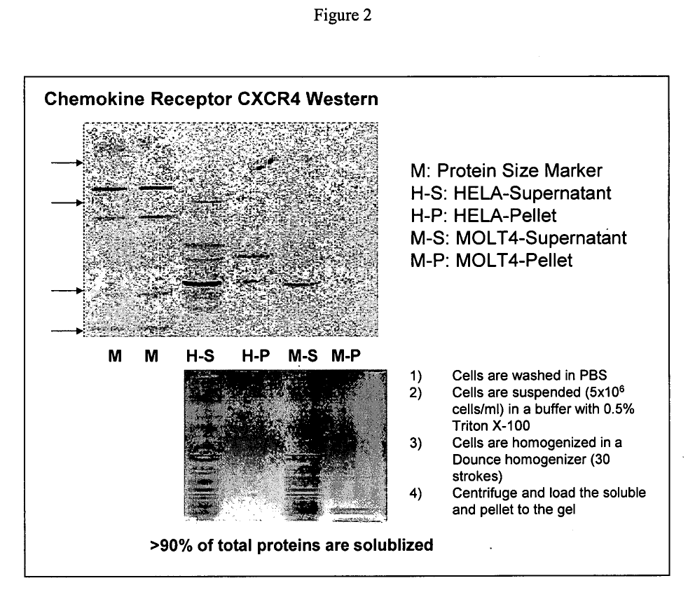

In the first step, a biological sample is obtained. The biological sample as used herein refers to any body sample such as blood (serum or plasma), sputum, ascites fluids, pleural effusions, urine, biopsy specimens, isolated cells and/or cell membrane preparation (see FIG. 2). Methods of obtaining tissue biopsies and body fluids from mammals are well known in the art.

Retrieved biological samples can be further solubilized using detergent-based or detergent free (i.e., sonication) methods, depending on the biological specimen and the nature of the examined polypeptide (i.e., secreted, membrane anchored or intracellular soluble polypeptide).

In certain embodiment, the sample may be denatured by detergent-free methods, such as thermo-denaturation. This is especially useful in applications where detergent needs to be removed or is preferably removed in future analysis.

In certain embodiments, the solubilized biological sample is contacted with one or more proteolytic agents. Digestion is effected under effective conditions and for a period of time sufficient to ensure complete digestion of the diagnosed polypeptide(s). Agents that are capable of digesting a biological sample under moderate conditions in terms of temperature and buffer stringency are preferred. Measures are taken not to allow non-specific sample digestion, thus the quantity of the digesting agent, reaction mixture conditions (i.e., salinity and acidity), digestion time and temperature are carefully selected. At the end of incubation time proteolytic activity is terminated to avoid non-specific proteolytic activity, which may evolve from elongated digestion period, and to avoid further proteolysis of other peptide-based molecules (i.e., protein-derived capture agents), which are added to the mixture in following steps.

If the sample is thermo-denatured, protease active at high temperatures, such as those isolated from thermophilic bacteria, can be used after the denaturation.

In the next method step the rendered biological sample is contacted with one or more capture agents, which are capable of discriminately binding one or more protein analytes through interaction via PET binding, and the products of such binding interactions examined and, as necessary, deconvolved, in order to identify and/or quantitate proteins found in the sample.

The present invention is based, at least in part, on the realization that unique recognition sequences (URSs) or PETs, which can be identified by computational analysis, can characterize individual proteins in a given sample, e.g., identify a particular protein from amongst others and/or identify a particular post-translationally modified form of a protein. The use of agents that bind PETs can be exploitated for the detection and quantitation of individual proteins from a milieu of several or many proteins in a biological sample. The subject method can be used to assess the status of proteins or protein modifications in, for example, bodily fluids, cell or tissue samples, cell lystates, cell membranes, etc. In certain embodiments, the method utilizes a set of capture agents which discriminate between splice variants, allelic variants and/or point mutations (e.g., altered amino acid sequences arising from single nucleotide polymorphisms).

As a result of the sample preparation, namely denaturation and/or proteolysis, the subject method can be used to detect specific proteins/modifications in a manner that does not require the homogeneity of the target protein for analysis and is relatively refractory to small but otherwise significant differences between samples. The methods of the invention are suitable for the detection of all or any selected subset of all proteins in a sample, including cell membrane bound and organelle membrane bound proteins.

In certain embodiments, the detection step(s) of the method are not sensitive to post-translational modifications of the native protein; while in other embodiments, the preparation steps are designed to preserve a post-translational modification of interest, and the detection step(s) use a set of capture agents able to discriminate between modified and unmodified forms of the protein. Exemplary post-translational modifications that the subject method can be used to detect and quantitate include acetylation, amidation, deamidation, prenylation (such as farnesylation or geranylation), formylation, glycosylation, hydroxylation, methylation, myristoylation, phosphorylation, ubiquitination, ribosylation and sulphation. In one specific embodiment, the phosphorylation to be assessed is phosphorylation on tyrosine, serine, threonine or histidine residue. In another specific embodiment, the addition of a hydrophobic group to be assessed is the addition of a fatty acid, e.g., myristate or palmitate, or addition of a glycosyl-phosphatidyl inositol anchor. In certain embodiment, the present method can be used to assess protein modification profile of a particular disease or disorder, such as infection, neoplasm (neoplasia), cancer, an immune system disease or disorder, a metabolism disease or disorder, a muscle and bone disease or disorder, a nervous system disease or disorder, a signal disease or disorder, or a transporter disease or disorder.

As used herein, the term “unique recognition sequence,” “URS,” “Proteome Epitope Tag,” or “PET” is intended to mean an amino acid sequence that, when detected in a particular sample (such as all proteins encoded by a genome or a subset thereof, etc.), unambiguously indicates that the protein from which it was derived is present in the sample. For instance, a PET is selected such that its presence in a sample, as indicated by detection of an authentic binding event with a capture agent designed to selectively bind with the sequence, necessarily means that the protein which comprises the sequence is present in the sample. A useful PET must present a binding surface that is solvent accessible, at least when a protein mixture is denatured and/or fragmented, and must bind with significant specificity to a selected capture agent with minimal cross reactivity. A unique recognition sequence or PET is present within the protein from which it is derived and in no other protein that may be present in the sample, cell type, or species under investigation. Moreover, a PET will preferably not have any closely related sequence (e.g., those with only 1, 2, or 3 amino acid sequence differences in a typical 8-10-amino acid PET sequence), such as determined by a nearest neighbor analysis, among the other proteins that may be present in the sample. A PET can be derived from a surface region of a protein, buried regions, splice junctions, or post translationally modified regions.

In some embodiments, the ideal PET is a peptide sequence which is present in only one protein in the proteome of a species. But a peptide comprising a PET useful in a human sample may in fact be present within the structure of proteins of other organisms. A PET useful in an adult cell sample is “unique” to that sample even though it may be present in the structure of other different proteins of the same organism at other times in its life, such as during embryonic development, or is present in other tissues or cell types different from the sample under investigation. A PET may be unique even though the same amino acid sequence is present in the sample from a different protein provided one or more of its amino acids are derivatized, and a binder can be developed which resolves the peptides.

In some embodiments, the ideal PET is a peptide sequence which is shared by the same protein (or a group of closely related proteins) from several different organisms, when it is of interest to, for example, determine the total amount of homologous proteins in the same sample, or to use the same PET (and its antibody) for samples from different species. Thus in this embodiment, it would be advantageous to select PETs such that one can detect the same set of proteins across different species. In other words, there will be no need to discriminate among the species since the sample may be restricted to a single species at a time. For example, when comparing biological mechanisms between animal models and human. Picking PETs that are shared in mouse, human and rat proteomes provides a practical advantage.

When referring herein to “uniqueness” with respect to a PET, the reference is always made in relation to the foregoing. Thus, within the human genome, a PET may be an amino acid sequence that is truly unique to the protein from which it is derived. Alternatively, it may be unique just to the sample from which it is derived, but the same amino acid sequence may be present in, for example, the murine genome. Likewise, when referring to a sample which may contain proteins from multiple different organism, uniqueness refers to the ability to unambiguously identify and discriminate between proteins from the different organisms, such as being from a host or from a pathogen.

Thus, a PET may be present within more than one protein in the species, provided it is unique to the sample from which it is derived. For example, a PET may be an amino acid sequence that is unique to: a certain cell type, e.g., a liver, brain, heart, kidney or muscle cell; a certain biological sample, e.g., a plasma, urine, amniotic fluid, genital fluid, marrow, spinal fluid, or pericardial fluid sample; a certain biological pathway, e.g., a G-protein coupled receptor signaling pathway or a tumor necrosis factor (TNF) signaling pathway.

In this sense, the instant invention provides a method to identify application-specific PETs, depending on the type of proteins present in a given sample. This information may be readily obtained from a variety of sources. For example, when the whole genome of an organism is concerned, the sequenced genome provides each and every protein sequences that can be encoded by this genome, sometimes even including hypothetical proteins. This “virtually translated proteome” obtained from the sequenced genome is expected to be the most comprehensive in terms of representing all proteins in the sample. Alternatively, the type of transcribed mRNA species (“virtually translated transcriptome”) within a sample may also provide useful information as to what type of proteins may be present within the sample. The mRNA species present may be identified by DNA microarrays, SNP analysis, or any other suitable RNA analysis tools available in the art of molecular biology. An added advantage of RNA analysis is that it may also provide information such as alternative splicing and mutations. Finally, direct protein analysis using techniques such as mass spectrometry may help to identify the presence of specific post-translation modifications and mutations, which may aid the design of specific PETs for specific applications. For example, WO 03/001879 A2 describes methods for determining the phosphorylaion status or sulfation state of a polypeptide or a cell using mass spectrometry, especially ICP-MS. In a related aspect, mass spectrometry, when coupled with separation techniques such as 2-D electrophoresis, GC/LC, etc., has provide a wealth of information regarding the profile of expressed proteins in specific samples.

For instance, plasma, the soluble component of the human blood, is believed to harbor thousands of distinct proteins, which originate from a variety of cells and tissues through either active secretion or leakage from blood cells or tissues. The dynamic range of plasma protein concentrations comprises at least nine orders of magnitude. Proteins involved in coagulation, immune defense, small molecule transport, and protease inhibition, many of them present in high abundance in this body fluid, have been functionally characterized and associated with disease processes. Pieper et al. (Proteomics 3: 1345-1364, 2003) fractionated blood serum proteins prior to display on two-dimensional electrophoresis (2-DE) gels using immunoaffinity chromatography to remove the most abundant serum proteins, followed by sequential anionexchange and size-exclusion chromatography. Serum proteins from 74 fractions were displayed on 2-DE gels. This approach succeeded in resolving approximately 3700 distinct protein spots, many of the post-translationally modified variants of plasma proteins. About 1800 distinct serum protein spots were identified by mass spectrometry. They collapsed into 325 distinct proteins, after sequence homology and similarity searches were carried out to eliminate redundant protein annotations. Coomassie Brillant Blue G-250 was used to visualize protein spots, and several proteins known to be present in serum in <10 ng/mL concentrations were identified such as interleukin-6, cathepsins, and peptide hormones.

The above article exemplifies a typical approach for MS-based protein profiling study. In a typical such study, proteins from a specific sample are first separated using a chosen appropriate method (such as 2-DE). To identify a separated protein, a gel spot or band is cut out, and in-gel tryptic digestion is performed thereafter. The gel must be stained with a mass spectrometry-compatible stain, for example colloidal Coommassie Brilliant Blue R-250 or Farmer's silver stain. The tryptic digest is then analyzed by MS such as MALDI-MS. The resulting mass spectrum of peptides, the peptide mass fingerprint or PMF, is searched against a sequence database. The PMF is compared to the masses of all theoretical tryptic peptides generated in silico by the search program. Programs such as Prospector, Sequest, and MasCot (Matrix Science, Ltd., London, UK) can be used for the database searching. For example, MasCot produces a statistically-based Mowse score indicates if any matches are significant or not. MS/MS is typically used to increase the likelihood of getting a database match. The PMF only contains the masses of the peptides. CID-MS/MS (collision induced dissociation of tandem MS) of peptides gives a spectrum of fragment ions that contain information about the amino-acid sequence. Adding this information to the peptide mass fingerprint allows Mascot to increase the statistical significance of a match. It is also possible in some cases to identify a protein by submitting only the raw MS/MS spectrum of a single peptide, a so-called MS/MS Ion Search, such is the amount of information contained in these spectra. MS/MS of peptides in a PMF can also greatly increase the confidence of a protein indentification, sometimes giving very high Mowse scores, especially with spectra from a TOF/TOF™.

Applied Biosystems 4700 Proteomics Analyzer, a MALDI-TOF/TOF™ tandem mass spectrometer, is unrivalled for the identification of proteins from tryptic digests, because of its sensitivity and speed. High-speed batch data acquisition is coupled to automated database searching using a locally-running copy of the Mascot search engine. When proteins cannot be identified by peptide mass mapping unambiguously, the digest can be further analyzed by a hybrid nanospray/ESI-Quadrupole-TOF-MS and MS/MS in a QSTAR mass spectrometer (Applied Biosystems Inc., Foster City, Calif.) for de novo peptide sequencing, sequence tag search, and/or MS/MS ion search. The static nanospray MS/MS is especially useful used when the target protein is not known (database absent). Applied Biosystems QSTAR® Pulsar i tandem mass spectrometer with a Dionex UltiMate capillary nanoLC system can be used for ES-LC-MS and MDLC (Multi-Dimensional Liquid Chromatography) analysis of peptide mixtures. A combination of these instruments can also perform MALDI-MS/MS, MDLC-ES-MS/MS, LC-MALDI, and Gel-C-MS/MS. With the Probot™ micro-fraction collector, HPLC can be interfaced with MALDI and spot peptides eluting from the nanoLC directly onto a MALDI target plate. This new LC-MALDI workflow for proteomics allows maximal potential for detecting proteins in complex mixtures by complementing the conventional 2-DE-based approach. For the traditional 2-DE approach, new and improved instruments, such as the Bio-Rad Protean 6-gel 2-DE apparatus and Packard MultiProbe II-EX robotic sample handler, in conjunction with the Applied Biosystems 4700 Proteomics Analyzer, allow higher sample throughputs for complete proteome characterizations.

Studies such as this, using equivalent instruments described above, have accumulated a large amount of MS data regarding expressed proteins and their specific protease digestion fragments, mostly tryptic fragment, stored in the form of many MS database. See, for example, MSDB (a non-identical protein sequence database maintained by the Proteomics Department at the Hammersmith Campus of Imperial College London. MSDB is designed specifically for mass spectrometry applications). PET analysis can be done on these tryptic peptides to identify PETs, which in turn is used for PET-specific antibody generation. The advantage of this approach is that it is known for certain that these (tryptic) peptide fragments will be generated in the sample of interest.

PETs identified based on the different methods described above may be combined. For example, in certain embodiments of the invention, multiple PETs need to be identified for any given target protein. Some of the PETs may be identified from sequenced genome data, while others may be identified from tryptic peptide databases.

The PET may be found in the native protein from which it is derived as a contiguous or as a non-contiguous amino acid sequence. It typically will comprise a portion of the sequence of a larger peptide or protein, recognizable by a capture agent either on the surface of an intact or partially degraded or digested protein, or on a fragment of the protein produced by a predetermined fragmentation protocol. The PET may be 5, 6, 7, 8, 9, 10, 11, 12, 13, 14, 15, 16, 17, 18, 19 or 20 amino acid residues in length. In a preferred embodiment, the PET is 6, 7, 8, 9 or 10 amino acid residues, preferably 8 amino acids in length.

The term “discriminate”, as in “capture agents able to discriminate between”, refers to a relative difference in the binding of a capture agent to its intended protein analyte and background binding to other proteins (or compounds) present in the sample. In particular, a capture agent can discriminate between two different species of proteins (or species of modifications) if the difference in binding constants is such that a statistically significant difference in binding is produced under the assay protocols and detection sensitivities. In preferred embodiments, the capture agent will have a discriminating index (D.I.) of at least 0.5, and even more preferably at least 0.1, 0.001, or even 0.0001, wherein D.I. is defined as K_{d}(a)/K_{d}(b), K_{d}(a) being the dissociation constant for the intended analyte, K_{d}(b) is the dissociation constant for any other protein (or modified form as the case may be) present in sample.

As used herein, the term “capture agent” includes any agent which is capable of binding to a protein that includes a unique recognition sequence, e.g., with at least detectable selectivity. A capture agent is capable of specifically interacting with (directly or indirectly), or binding to (directly or indirectly) a unique recognition sequence. The capture agent is preferably able to produce a signal that may be detected. In a preferred embodiment, the capture agent is an antibody or a fragment thereof, such as a single chain antibody, or a peptide selected from a displayed library. In other embodiments, the capture agent may be an artificial protein, an RNA or DNA aptamer, an allosteric ribozyme or a small molecule. In other embodiments, the capture agent may allow for electronic (e.g., computer-based or information-based) recognition of a unique recognition sequence. In one embodiment, the capture agent is an agent that is not naturally found in a cell.

As used herein, the term “globally detecting” includes detecting at least 40% of the proteins in the sample. In a preferred embodiment, the term “globally detecting” includes detecting at least 50%, 60%, 65%, 70%, 75%, 80%, 85%, 90%, 95% or 100% of the proteins in the sample. Ranges intermediate to the above recited values, e.g., 50%-70% or 75%-95%, are also intended to be part of this invention. For example, ranges using a combination of any of the above recited values as upper and/or lower limits are intended to be included.

As used herein, the term “proteome” refers to the complete set of chemically distinct proteins found in an organism.

As used herein, the term “organism” includes any living organism including animals, e.g., avians, insects, mammals such as humans, mice, rats, monkeys, or rabbits; microorganisms such as bacteria, yeast, and fungi, e.g., Escherichia coli, Campylobacter, Listeria, Legionella, Staphylococcus, Streptococcus, Salmonella, Bordatella, Pneumococcus, Rhizobium, Chlamydia, Rickettsia, Streptomyces, Mycoplasma, Helicobacter pylori, Chlamydia pneumoniae, Coxiella burnetii, Bacillus Anthracis, and Neisseria; protozoa, e.g., Trypanosoma brucei; viruses, e.g., human immunodeficiency virus, rhinoviruses, rotavirus, influenza virus, Ebola virus, simian immunodeficiency virus, feline leukemia virus, respiratory syncytial virus, herpesvirus, pox virus, polio virus, parvoviruses, Kaposi's Sarcoma-Associated Herpesvirus (KSHV), adeno-associated virus (AAV), Sindbis virus, Lassa virus, West Nile virus, enteroviruses, such as 23 Coxsackie A viruses, 6 Coxsackie B viruses, and 28 echoviruses, Epstein-Barr virus, caliciviruses, astroviruses, and Norwalk virus; fungi, e.g., Rhizopus, neurospora, yeast, or puccinia; tapeworms, e.g., Echinococcus granulosus, E. multilocularis, E. vogeli and E. oligarthrus; and plants, e.g., Arabidopsis thaliana, rice, wheat, maize, tomato, alfalfa, oilseed rape, soybean, cotton, sunflower or canola.

As used herein, “sample” refers to anything which may contain a protein analyte. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregates of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s). The sample may also be a mixture of target protein containing molecules prepared in vitro.

As used herein, “a comparable control sample” refers to a control sample that is only different in one or more defined aspects relative to a test sample, and the present methods, kits or arrays are used to identify the effects, if any, of these defined difference(s) between the test sample and the control sample, e.g., on the amounts and types of proteins expressed and/or on the protein modification profile. For example, the control biosample can be derived from physiological normal conditions and/or can be subjected to different physical, chemical, physiological or drug treatments, or can be derived from different biological stages, etc.

“Predictably result from a treatment” means that a peptide fragment can be reliably generated by certain treatments, such as site specific protease digestion or chemical fragmentation. Since the digestion sites are quite specific, the peptide fragment generated by specific treatments can be reliably predicted in silico.

A report by MacBeath and Schreiber (Science 289 (2000), pp. 1760-1763) in 2000 established that proteins could be printed and assayed in a microarray format, and thereby had a large role in renewing the excitement for the prospect of a protein chip. Shortly after this, Snyder and co-workers reported the preparation of a protein chip comprising nearly 6000 yeast gene products and used this chip to identify new classes of calmodulin- and phospholipid-binding proteins (Zhu et al., Science 293 (2001), pp. 2101-2105). The proteins were generated by cloning the open reading frames and overproducing each of the proteins as glutathione-S-transferase-(GST) and His-tagged fusions. The fusions were used to facilitate the purification of each protein and the His-tagged family were also used in the immobilization of proteins. This and other references in the art established that microarrays containing thousands of proteins could be prepared and used to discover binding interactions. They also reported that proteins immobilized by way of the His tag—and therefore uniformly oriented at the surface—gave superior signals to proteins randomly attached to aldehyde surfaces.

Related work has addressed the construction of antibody arrays (de Wildt et al., Antibody arrays for high-throughput screening of antibody-antigen interactions. Nat. Biotechnol. 18 (2000), pp. 989-994; Haab, B. B. et al. (2001) Protein microarrays for highly parallel detection and quantitation of specific proteins and antibodies in complex solutions. Genome Biol. 2, RESEARCH0004.1-RESEARCH0004.13). Specifically, in an early landmark report, de Wildt and Tomlinson immobilized phage libraries presenting scFv antibody fragments on filter paper to select antibodies for specific antigens in complex mixtures (supra). The use of arrays for this purpose greatly increased the throughput when evaluating antibodies, allowing nearly 20,000 unique clones to be screened in one cycle. Brown and co-workers extended this concept to create molecularly defined arrays wherein antibodies were directly attached to aldehyde-modified glass. They printed 115 commercially available antibodies and analyzed their interactions with cognate antigens with semi-quantitative results (supra). Kingsmore and co-workers used an analogous approach to prepare arrays of antibodies recognizing 75 distinct cytokines and, using the rolling-circle amplification strategy (Lizardi et al., Mutation detection and single molecule counting using isothermal rolling circle amplification. Nat. Genet. 19 (1998), pp. 225-233), could measure cytokines at femtomolar concentrations (Schweitzer et al., Multiplexed protein profiling on microarrays by rolling-circle amplification. Nat. Biotechnol. 20 (2002), pp. 359-365).

There are at least two array formats that can be used in competition assays for analyte concentration measurement.

In one embodiment (the PET peptide array), the method utilizes an array of peptide fragments immobilized on a support, the array comprising a plurality of peptide fragments, each of which represents one unique target protein within the sample. The peptide fragments each contain a PET sequence unique within the sample. When such an array is in contact with a mixture of capture agents specific for the immobilized peptides, the capture agents will specifically bind to their respective immobilized peptide fragments. Ideally, each capture agent only binds the peptide against which the capture agent is raised, but not any other peptides on the same array (e.g., no cross-reactivity). However, if soluble competition peptides are added to the binding mixture, the amount of capture agents remaining bound to the immobilized peptide fragments will be accordingly reduced, depending on the amount/concentration of soluble competition peptides in the binding mixture. A standard curve for each specific target protein may be generated based on the amount of soluble competition peptides within the binding mixture, and the amount of capture agents remaining bound to the immobilized PET-containing peptide fragment on the array. Such a standard curve may be used to determine the amount of that target protein in an unknown sample. The method may also be used to simultaneously quantitate more than one target proteins within the sample, by generating a standard competition curve for each of the many target proteins. In this embodiment, the capture agents are usually labeled (e.g. fluorescent dye) for detection. The same label can be used for different capture agents in the same reaction if there is virtually no cross-reactivity.

In an alternative embodiment (the capture agent array), an array of capture agents are immobilized on a support. Each of the capture agents is specific for a given PET-containing peptide fragment within a sample. When such an array is in contact with a treated sample with the target PET-containing peptides of the capture agents, the PET-containing peptides will be bound by the capture agents. However, if a labeled competition PET-containing peptide is also present in the binding mixture, the labeled and unlabeled PET-containing peptides will compete for binding to the capture agent, in a concentration dependent manner. The amount of labeled PET-containing peptides bound to the immobilized capture agents will depend on the concentration of the competing unlabeled PET-containing peptides. Thus, a standard competition curve can be established by using a known concentration of labeled PET-containing peptide and a series of known concentrations of unlabeled PET-containing peptides. This standard curve can then be used to measure the concentration of the target PET-containing peptide in the sample. The method may also be used to simultaneously quantitate more than one target proteins within the sample, by generating a standard competition curve for each of the many target proteins. The same (or different) label can be used for different target peptides since their respective capture agents are located on distinct addressable locations on the support, and thus the same kind of signal can be readily distinguished by their locations on the support (array). In this embodiment, the peptides are usually labeled for detection.

When assessing expression profile of the same analytes in two (or more) different samples, it may be useful to obtain a quantitative readout for each protein that is being measured, as well as a differential assessment between protein levels between two samples. Gene chips have set the standard on differential measurement, where two different labels (typically fluorescent dyes) are incorporated into two different samples to be measured (each sample gets its own label). The relative gene expression between these two samples can be determined. In this way, one can compare, for example, “normal” samples with “disease” samples. For quantification of each gene, specific probes may be used to amplify and analyze the signal by quantitative PCR.

A similar approach may be adapted for differential protein assessment. The main advantages of the differential approach are: a) no need to provide a standard curve for each analyte; and, b) ability to handle a large dynamic range, as even abundant proteins, which on their own would saturate their antibodies and hence be out of range, are measurable when two samples are analyzed simultaneously. The amount of each differently labeled protein is below the saturation level of the antibody. The relative amount of each dye bound to the antibody reflects the amount of protein in the starting sample. In this way, one determines the relative expression of protein between one sample and another (e.g. two fold higher). The downside of the differential measurement is that there is no reliable way to compare results generated in different labs or between samples analyzed on different days, unless exactly the same reference sample is used and the sample needs to be labeled prior to analysis.

On the other hand, quantitative assays are routinely employed for immunoassays. In this type of assay, an assay standard is provided with the assay kit and a standard curve is generated as part of each measurement. The subject antibody design approach (e.g. the PET peptide antibodies) provides the level of selectivity needed to minimize antibody cross-talk when multiple types of antibodies are used in the same assay.

The two assay platforms described above (either peptide array or antibody array) both provide a quantification standard curve for each antibody/antigen (e.g. peptide) pair. The standard curve may be constructed for all analytes (e.g. peptides) simultaneously, using several sample chambers on an array (e.g. a slide), while the remaining chambers can be used for different samples to be analyzed. Each chamber typically contains the same printing pattern of immobilized antigens or antibodies.

In certain embodiments, an improvement of the assay platforms combine aspects of both the differential and quantitative assay into one format, allowing capturing the benefits of both. For example, one labeling reagent may be used to label all the peptide standards (for example, using green dye for standard peptides 1, 2, and 3 to be measured). Meanwhile, a second, different labeling reagent (e.g. red dye) is used to label the sample to be measured. A mixture of the labeled peptide standards is provided in the assay kit at a known and predetermined concentration. The assay standard cocktail is combined with the labeled sample and applied to a single chamber that contains the immobilized antibody array. Each antibody in the chamber is consequently labeled with both dyes, where the quantity of the dyes reflects the relative amount of the analyte (e.g., peptide fragment containing the PET) between the peptide standard and the unknown sample. The data obtained may be reported in differential terms (e.g. “2 fold higher than standard” etc.) or in absolute terms (e.g. 0.01 mg/ml, etc.), since the concentration of each standard used is known. Since all results are calibrated to the standard provided, results can be compared across all measurements. This seeming straightforward approach is uniquely suited to the subject PET-based approach, since it is not practical to provide labeled whole proteins as standards due to complexities such as generating the whole proteins in the first place, and then keeping the labeled proteins stable. In addition, the total concentration of proteins in the labeled standard would be many folds higher (likely 10-100 fold higher) if whole proteins (instead of small PET-peptides) are used, practically limiting the number of standard peptides that may be included in the same reaction.

The benefits of this assay format include at least the following:
higher throughput—more chambers on each array/slide can be dedicated to samples, rather than being used to construct standard curves.
broader dynamic range—the low end of the detection range is determined by antibody affinity (k_{d}) and background relative to signal. The high end of the range is essentially infinite as long as the unknown sample and peptide standard can adequately compete for binding (e.g. one amount is not orders of magnitude greater than the other). User can adjust the concentration of the labeled peptide standard in their measurement to select the appropriate range for that sample. User can also adjust detector (e.g. PMT) settings to match the readout for each antibody within each sample chamber.
ability to accommodate chamber to chamber differences—it can be shown that the relative binding between two samples is insensitive to variability in antibody performance chamber to chamber, as any chamber-specific changes impact both the sample and the standard equally (the advantage of internal control). For the same reason, this assay format will be able to accommodate differences in antibody affinity between different lots of antibodies. Thus this assay represents a much more forgiving approach.

These examples demonstrate the many important roles that protein chips can play, and give evidence for the widespread activity in fabrication of these tools. The following subsections describes in further detail about various aspects of the invention.

I. Type of Capture Agents

In certain preferred embodiments, the capture agents used should be capable of selective affinity reactions with PET moieties. Generally, such ineraction will be non-covalent in nature, though the present invention also contemplates the use of capture reagents that become covalently linked to the PET.

Examples of capture agents which can be used include, but are not limited to: nucleotides; nucleic acids including oligonucleotides, double stranded or single stranded nucleic acids (linear or circular), nucleic acid aptamers and ribozymes; PNA (peptide nucleic acids); proteins, including antibodies (such as monoclonal or recombinantly engineered antibodies or antibody fragments), T cell receptor and MHC complexes, lectins and scaffolded peptides; peptides; other naturally occurring polymers such as carbohydrates; artificial polymers, including plastibodies; small organic molecules such as drugs, metabolites and natural products; and the like.

In certain embodiments, the capture agents are immobilized, permanently or reversibly, on a solid support such as a bead, chip, or slide. When employed to analyze a complex mixture of proteins, the immobilized capture agent are arrayed and/or otherwise labeled for deconvolution of the binding data to yield identity of the capture agent (and therefore of the protein to which it binds) and (optionally) to quantitate binding. Alternatively, the capture agents can be provided free in solution (soluble), and other methods can be used for deconvolving PET binding in parallel.

In one embodiment, the capture agents are conjugated with a reporter molecule such as a fluorescent molecule or an enzyme, and used to detect the presence of bound PET on a substrate (such as a chip or bead), in for example, a “sandwich” type assay in which one capture agent is immobilized on a support to capture a PET, while a second, labeled capture agent also specific for the captured PET may be added to detect/quantitate the captured PET. In this embodiment, the peptide fragment contains two unique, non-overlapping PETs, one recognized by the immobilized the capture agent, the other recognized by the labled detecting capture agent. In a related embodiment, one PET unique to the peptide fragment can be used in conjunction with a common PET shared among several protein family members. The spacial arrangement of these two PET is such that binding by one capture agent will not substantially affect the binding by the other capture agent. In addition, the length of the peptide fragment is such that it encompasses two PETs properly spaced from each other. Preferably, peptide fragments is at least about 15 residues for sandwich assay. In other embodiments a labeled-PET peptide is used in a competitive binding assay to determine the amount of unlabeled PET (from the sample) binds to the capture agent. In this embodiment, the peptide fragment need only be long enough to encompass one PET, so peptides as short as 5-8 residues may be suitable.

Generally, the sandwich assay tend to be more (e.g., about 10, 100, or 1000 fold more) sensitive than the competitive binding assay.

An important advantage of the invention is that useful capture agents can be identified and/or synthesized even in the absence of a sample of the protein to be detected. With the completion of the whole genome in a number of organisms, such as human, fly (Drosophila melanogaster) and nematode (C. elegans), PET of a given length or combination thereof can be identified for any single given protein in a certain organism, and capture agents for any of these proteins of interest can then be made without ever cloning and expressing the full length protein.

In addition, the suitability of any PET to serve as an antigen or target of a capture agent can be further checked against other available information. For example, since amino acid sequence of many proteins can now be inferred from available genomic data, sequence from the structure of the proteins unique to the sample can be determined by computer aided searching, and the location of the peptide in the protein, and whether it will be accessible in the intact protein, can be determined. Once a suitable PET peptide is found, it can be synthesized using known techniques. With a sample of the PET in hand, an agent that interacts with the peptide such as an antibody or peptidic binder, can be raised against it or panned from a library. In this situation, care must be taken to assure that any chosen fragmentation protocol for the sample does not restrict the protein in a way that destroys or masks the PET. This can be determined theoretically and/or experimentally, and the process can be repeated until the selected PET is reliably retrieved by a capture agent(s).

The PET set selected according to the teachings of the present invention can be used to generate peptides either through enzymatic cleavage of the protein from which they were generated and selection of peptides, or preferably through peptide synthesis methods.

Proteolytically cleaved peptides can be separated by chromatographic or electrophoretic procedures and purified and renatured via well known prior art methods.

Synthetic peptides can be prepared by classical methods known in the art, for example, by using standard solid phase techniques. The standard methods include exclusive solid phase synthesis, partial solid phase synthesis methods, fragment condensation, classical solution synthesis, and even by recombinant DNA technology. See, e.g., Merrifield, J. Am. Chem. Soc., 85:2149 (1963), incorporated herein by reference. Solid phase peptide synthesis procedures are well known in the art and further described by John Morrow Stewart and Janis Dillaha Young, Solid Phase Peptide Syntheses (2nd Ed., Pierce Chemical Company, 1984).

Synthetic peptides can be purified by preparative high performance liquid chromatography [Creighton T. (1983) Proteins, structures and molecular principles. WH Freeman and Co. N.Y.] and the composition of which can be confirmed via amino acid sequencing.

In addition, other additives such as stabilizers, buffers, blockers and the like may also be provided with the capture agent.

A. Antibodies

In one embodiment, the capture agent is an antibody or an antibody-like molecule (collectively “antibody”). Thus an antibody useful as capture agent may be a full length antibody or a fragment thereof, which includes an “antigen-binding portion” of an antibody. The term “antigen-binding portion,” as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al. (1988) Science 242:423-426; and Huston et al. (1988) Proc. Natl. Acad. Sci. USA 85:5879-5883; and Osbourn et al. 1998, Nature Biotechnology 16: 778). Such single chain antibodies are also intended to be encompassed within the term “antigen-binding portion” of an antibody. Any V_{H} and V_{L} sequences of specific scFv can be linked to human immunoglobulin constant region cDNA or genomic sequences, in order to generate expression vectors encoding complete IgG molecules or other isotypes. V_{H} and V_{L} can also be used in the generation of Fab, Fv or other fragments of immunoglobulins using either protein chemistry or recombinant DNA technology. Other forms of single chain antibodies, such as diabodies are also encompassed. Diabodies are bivalent, bispecific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see, e.g., Holliger, P., et al. (1993) Proc. Natl. Acad. Sci. USA 90:6444-6448; Poljak, R. J., et al. (1994) Structure 2:1121-1123).

Still further, an antibody or antigen-binding portion thereof may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques.

Antibodies may be polyclonal or monoclonal. The terms “monoclonal antibodies” and “monoclonal antibody composition,” as used herein, refer to a population of antibody molecules that contain only one species of an antigen binding site capable of immunoreacting with a particular epitope of an antigen, whereas the term “polyclonal antibodies” and “polyclonal antibody composition” refer to a population of antibody molecules that contain multiple species of antigen binding sites capable of interacting with a particular antigen. A monoclonal antibody composition, typically displays a single binding affinity for a particular antigen with which it immunoreacts.

Any art-recognized methods can be used to generate an PET-directed antibody. For example, a PET (alone or linked to a hapten) can be used to immunize a suitable subject, (e.g., rabbit, goat, mouse or other mammal or vertebrate). For example, the methods described in U.S. Pat. Nos. 5,422,110; 5,837,268; 5,708,155; 5,723,129; and 5,849,531 (the contents of each of which are incorporated herein by reference) can be used. The immunogenic preparation can further include an adjuvant, such as Freund's complete or incomplete adjuvant, or similar immunostimulatory agent. Immunization of a suitable subject with a PET induces a polyclonal anti-PET antibody response. The anti-PET antibody titer in the immunized subject can be monitored over time by standard techniques, such as with an enzyme linked immunosorbent assay (ELISA) using immobilized PET.

The antibody molecules directed against a PET can be isolated from the mammal (e.g., from the blood) and further purified by well known techniques, such as protein A chromatography to obtain the IgG fraction. At an appropriate time after immunization, e.g., when the anti-PET antibody titers are highest, antibody-producing cells can be obtained from the subject and used to prepare, e.g., monoclonal antibodies by standard techniques, such as the hybridoma technique originally described by Kohler and Milstein (1975) Nature 256:495-497) (see also, Brown et al. (1981) J. Immunol. 127:53946; Brown et al. (1980) J. Biol. Chem 0.255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol Today 4:72), or the EBV-hybridoma technique (Cole et al. (1985), Monoclonal Antibodies and Cancer Therapy, Alan R. Liss, Inc., pp. 77-96). The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with a PET immunogen as described above, and the culture supernatants of the resulting hybridoma cells are screened to identify a hybridoma producing a monoclonal antibody that binds a PET.

Any of the many well known protocols used for fusing lymphocytes and immortalized cell lines can be applied for the purpose of generating an anti-PET monoclonal antibody (see, e.g., G. Galfre et al. (1977) Nature 266:55052; Gefter et al. Somatic Cell Genet., cited supra; Lerner, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O—Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind a PET, e.g., using a standard ELISA assay.

In addition, automated screening of antibody or scaffold libraries against arrays of target proteins/PETs will be the most rapid way of developing thousands of reagents that can be used for protein expression profiling. Furthermore, polyclonal antisera, hybridomas or selection from library systems may also be used to quickly generate the necessary capture agents. A high-throughput process for antibody isolation is described by Hayhurst and Georgiou in Curr Opin Chem Biol 5(6):683-9, December 2001 (incorporated by reference).

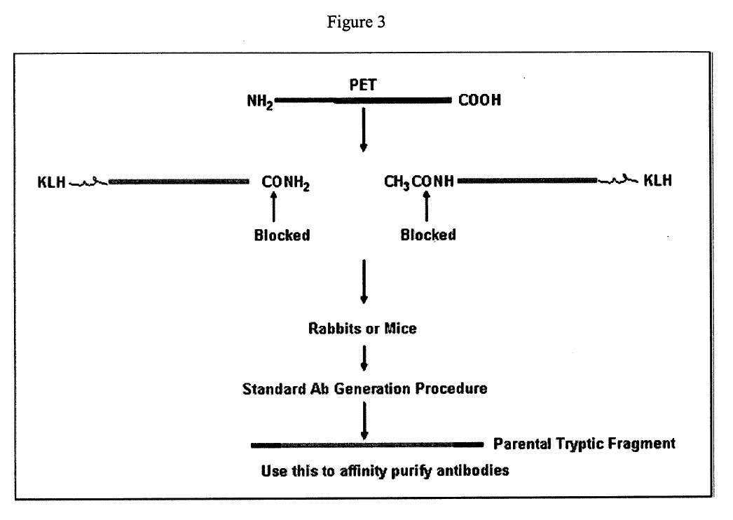

The PET antigens (either synthesized or natural) used for the generation of PET-specific antibodies are preferably blocked at either the N- or C-terminal end, most preferably at both ends (see FIG. 3) to generate neutral groups, since antibodies raised against peptides with non-neutralized ends may not be functional for the methods of the invention. The PET antigens can be most easily synthesized using standard molecular biology or chemical methods, for example, with a peptide synthesizer. The terminals can be blocked with NH₂— or COO— groups as appropriate, or any other blocking agents to eliminate free ends. In a preferred embodiment, one end (either N- or C-terminus) of the PET will be conjugated with a carrier protein such as KLH or BSA to facilitate antibody generation. KLH represents Keyhole-limpet hemocyanin, an oxygen carrying copper protein found in the keyhole-limpet (Megathura crenulata), a primitive mollusk sea snail. KLH has a complex molecular arrangement and contains a diverse antigenic structure and elicits a strong nonspecific immune response in host animals. Therefore, when small peptides (which may not be very immunogenic) are used as immunogens, they are preferably conjugated to KLH or other carrier proteins (BSA) for enhanced immune responses in the host animal. The resulting antibodies can be affinity purified using a polypeptide corresponding to the PET-containing tryptic peptide of interest (see FIG. 3).

Blocking the ends of PET in antibody generation may be advantageous, since in many (if not most) cases, the selected PETs are contained within larger (tryptic) fragments. In these cases, the PET-specific antibodies are required to bind PETs in the middle of a peptide fragment. Therefore, blocking both the C- and N-terminus of the PETs best simulates the antibody binding of peptide fragments in a digested sample. Similarly, if the selected PET sequence happens to be at the N- or C-terminal end of a target fragment, then only the other end of the immunogen needs to be blocked, preferably by a carrier such as KLH or BSA.

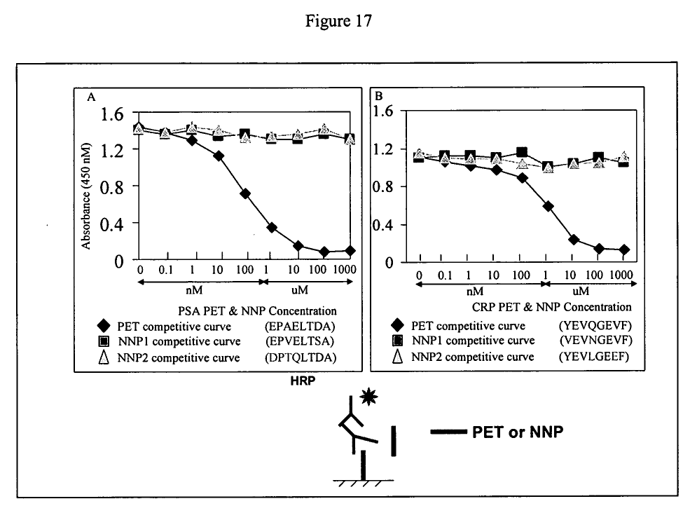

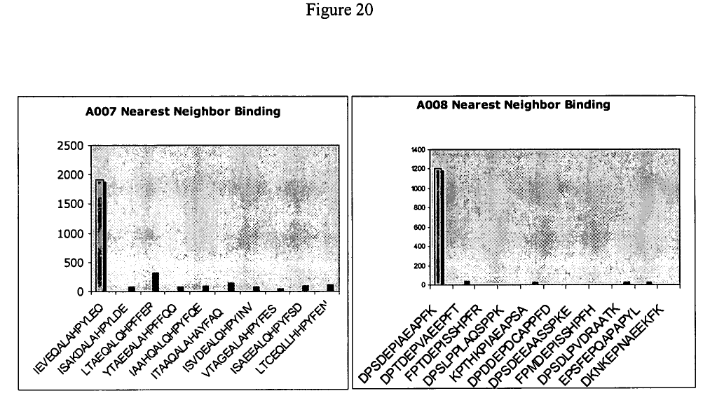

FIGS. 17 and 20 below show that PET-specific antibodies are highly specific and have high affinity for their respective PET-antigens.

When generating PET-specific antibodies, preferably monoclonal antibodies, a peptide immunogen comprising essentially of the target PET sequence may be administered to an animal according to standard antibody generation protocol for short peptide antigens. In one embodiment, the short peptide antigen may be conjugated with a carrier such as KLH. However, when screening for antibodies specific for the PET sequence, it is preferred that the parental peptide fragments containing the PET sequence (such as the fragment resulting from trypsin digestion) is used. This ensures that the identified antibodies will be not only specific for the original PET sequence, but also able to recognize the PET peptide fragment for which the antibody is designed. Optionally, the specificity of the identified antibody can be further verified by reacting with the original immunogen such as the end-blocked PET sequence itself.

In certain embodiments, several different immunogens for different PET sequences may be simultaneously administered to the same animal, so that different antibodies may be generated in one animal. Obviously, for each immunogen, a separate screen would be needed to identify antibodies specific for the immunogen.

In an alternative embodiment, different PETs may be linked together in a single, longer immunogen for administration to an animal. The linker sequence can be flexible linkers such as GS, GSSSS or repeats thereof (such as three-peats).

In both embodiments described above, the different immunogens may be from the same or different organisms or proteomes. These methods are all potential means of reducing costs in antibody generation. An unexpected advantage of using linked PET sequences as immunogen is that longer immunogens may at certain situations produce higher affinity antibodies than those produced using short PET sequences.

(i) PET-Specific Antibody Knowledge Database

The instant invention also provides an antibody knowledge database, which provides various important information pertaining to these antibodies. A specific subset of the antibodies will be PET-specific antibodies, which are either generated de novo based on the criteria set forth in the instant application, or generated by others in the prior art, which happens to recognize certain PETs.

Information to be included in the knowledge database can be quite comprehensive. Such knowledge may be further classified as public or proprietary. Examples of public information may include: target protein name, antibody source, catalog number, potential applications, etc. Exemplary proprietary information includes parental tryptic fragments in one or more organisms or specific samples, immunogen peptide sequences and whether or not they are PETs, affinity for the target PET, degree of cross-reactivity with other related epitopes (such as the closest nearest neighbors), and usefulness for various PET assays.

To this end, such information about 1000 anti-peptide antibodies are already collected/generated in the knowledge database. Among them, about 128 antibodies are deemed compatible for trypsin digested samples. Certain commercially available antibodies include: Anti-Cyclin F, Anti-phospho SHC (Tyr239), Anti-phospho-PP2A (Tyr307), and Anti-Cdk8, the immunogen and the PET sequences they happen to contain, and the nearest neighbors of these PETs are listed in the parent applications (incorporated herein by reference).

B. Proteins and Peptides

Other methods for generating the capture agents of the present invention include phage-display technology described in, for example, Dower et al., WO 91/17271, McCafferty et al., WO 92/01047, Herzig et al., U.S. Pat. No. 5,877,218, Winter et al., U.S. Pat. No. 5,871,907, Winter et al., U.S. Pat. No. 5,858,657, Holliger et al., U.S. Pat. No. 5,837,242, Johnson et al., U.S. Pat. No. 5,733,743 and Hoogenboom et al., U.S. Pat. No. 5,565,332 (the contents of each of which are incorporated by reference). In these methods, libraries of phage are produced in which members display different antibodies, antibody binding sites, or peptides on their outer surfaces. Antibodies are usually displayed as Fv or Fab fragments. Phage displaying sequences with a desired specificity are selected by affinity enrichment to a specific PET.

Methods such as yeast display and in vitro ribosome display may also be used to generate the capture agents of the present invention. The foregoing methods are described in, for example, Methods in Enzymology Vol 328-Part C: Protein-protein interactions & Genomics and Bradbury A. (2001) Nature Biotechnology 19:528-529, the contents of each of which are incorporated herein by reference.

In a related embodiment, proteins or polypeptides may also act as capture agents of the present invention. These peptide capture agents also specifically bind to an given PET, and can be identified, for example, using phage display screening against an immobilized PET, or using any other art-recognized methods. Once identified, the peptidic capture agents may be prepared by any of the well known methods for preparing peptidic sequences. For example, the peptidic capture agents may be produced in prokaryotic or eukaryotic host cells by expression of polynucleotides encoding the particular peptide sequence. Alternatively, such peptidic capture agents may be synthesized by chemical methods. Methods for expression of heterologous peptides in recombinant hosts, chemical synthesis of peptides, and in vitro translation are well known in the art and are described further in Maniatis et al., Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91:501; Chaiken, I. M. (1981) CRC Crit. Rev. Biochem. 11:255; Kaiser et al. (1989) Science 243:187; Merrifield, B. (1986) Science 232:342; Kent, S. B. H. (1988) Ann. Rev. Biochem. 57:957; and Offord, R. E. (1980) Semisynthetic Proteins, Wiley Publishing, which are incorporated herein in their entirety by reference).

The peptidic capture agents may also be prepared by any suitable method for chemical peptide synthesis, including solution-phase and solid-phase chemical synthesis. Preferably, the peptides are synthesized on a solid support. Methods for chemically synthesizing peptides are well known in the art (see, e.g., Bodansky, M. Principles of Peptide Synthesis, Springer Verlag, Berlin (1993) and Grant, G. A (ed.). Synthetic Peptides: A User's Guide, W.H. Freeman and Company, New York (1992). Automated peptide synthesizers useful to make the peptidic capture agents are commercially available.

C. Scaffolded Peptides

An alternative approach to generating capture agents for use in the present invention makes use of antibodies are scaffolded peptides, e.g., peptides displayed on the surface of a protein. The idea is that restricting the degrees of freedom of a peptide by incorporating it into a surface-exposed protein loop could reduce the entropic cost of binding to a target protein, resulting in higher affinity. Thioredoxin, fibronectin, avian pancreatic polypeptide (aPP) and albumin, as examples, are small, stable proteins with surface loops that will tolerate a great deal of sequence variation. To identify scaffolded peptides that selectively bind a target PET, libraries of chimeric proteins can be generated in which random peptides are used to replace the native loop sequence, and through a process of affinity maturation, those which selectively bind a PET of interest are identified.

D. Simple Peptides and Peptidomimetic Compounds

Peptides are also attractive candidates for capture agents because they combine advantages of small molecules and proteins. Large, diverse libraries can be made either biologically or synthetically, and the “hits” obtained in binding screens against PET moieties can be made synthetically in large quantities.

Peptide-like oligomers (Soth et al. (1997) Curr. Opin. Chem. Biol. 1:120-129) such as peptoids (Figliozzi et al., (1996) Methods Enzymol. 267:437-447) can also be used as capture reagents, and can have certain advantages over peptides. They are impervious to proteases and their synthesis can be simpler and cheaper than that of peptides, particularly if one considers the use of functionality that is not found in the 20 common amino acids.

E. Nucleic Acids

In another embodiment, aptamers binding specifically to a PET may also be used as capture agents. As used herein, the term “aptamer,” e.g., RNA aptamer or DNA aptamer, includes single-stranded oligonucleotides that bind specifically to a target molecule. Aptamers are selected, for example, by employing an in vitro evolution protocol called systematic evolution of ligands by exponential enrichment. Aptamers bind tightly and specifically to target molecules; most aptamers to proteins bind with a K_{d} (equilibrium dissociation constant) in the range of 1 pM to 1 nM. Aptamers and methods of preparing them are described in, for example, E. N. Brody et al. (1999) Mol. Diagn. 4:381-388, the contents of which are incorporated herein by reference.

In one embodiment, the subject aptamers can be generated using SELEX, a method for generating very high affinity receptors that are composed of nucleic acids instead of proteins. See, for example, Brody et al. (1999) Mol. Diagn. 4:381-388. SELEX offers a completely in vitro combinatorial chemistry alternative to traditional protein-based antibody technology. Similar to phage display, SELEX is advantageous in terms of obviating animal hosts, reducing production time and labor, and simplifying purification involved in generating specific binding agents to a particular target PET.

To further illustrate, SELEX can be performed by synthesizing a random oligonucleotide library, e.g., of greater than 20 bases in length, which is flanked by known primer sequences. Synthesis of the random region can be achieved by mixing all four nucleotides at each position in the sequence. Thus, the diversity of the random sequence is maximally 4ⁿ, where n is the length of the sequence, minus the frequency of palindromes and symmetric sequences. The greater degree of diversity conferred by SELEX affords greater opportunity to select for oligonuclotides that form 3-dimensional binding sites. Selection of high affinity oligonucleotides is achieved by exposing a random SELEX library to an immobilized target PET. Sequences, which bind readily without washing away, are retained and amplified by the PCR, for subsequent rounds of SELEX consisting of alternating affinity selection and PCR amplification of bound nucleic acid sequences. Four to five rounds of SELEX are typically sufficient to produce a high affinity set of aptamers.

Therefore, hundreds to thousands of aptamers can be made in an economically feasible fashion. Blood and urine can be analyzed on aptamer chips that capture and quantitate proteins. SELEX has also been adapted to the use of 5-bromo (5-Br) and 5-iodo (5-I) deoxyuridine residues. These halogenated bases can be specifically cross-linked to proteins. Selection pressure during in vitro evolution can be applied for both binding specificity and specific photo-cross-linkability. These are sufficiently independent parameters to allow one reagent, a photo-cross-linkable aptamer, to substitute for two reagents, the capture antibody and the detection antibody, in a typical sandwich array. After a cycle of binding, washing, cross-linking, and detergent washing, proteins will be specifically and covalently linked to their cognate aptamers. Because no other proteins are present on the chips, protein-specific stain will now show a meaningful array of pixels on the chip. Combined with learning algorithms and retrospective studies, this technique should lead to a robust yet simple diagnostic chip.

In yet another related embodiment, a capture agent may be an allosteric ribozyme. The term “allosteric ribozymes,” as used herein, includes single-stranded oligonucleotides that perform catalysis when triggered with a variety of effectors, e.g., nucleotides, second messengers, enzyme cofactors, pharmaceutical agents, proteins, and oligonucleotides. Allosteric ribozymes and methods for preparing them are described in, for example, S. Seetharaman et al. (2001) Nature Biotechnol. 19: 336-341, the contents of which are incorporated herein by reference. According to Seetharaman et al., a prototype biosensor array has been assembled from engineered RNA molecular switches that undergo ribozyme-mediated self-cleavage when triggered by specific effectors. Each type of switch is prepared with a 5′-thiotriphosphate moiety that permits immobilization on gold to form individually addressable pixels. The ribozymes comprising each pixel become active only when presented with their corresponding effector, such that each type of switch serves as a specific analyte sensor. An addressed array created with seven different RNA switches was used to report the status of targets in complex mixtures containing metal ion, enzyme cofactor, metabolite, and drug analytes. The RNA switch array also was used to determine the phenotypes of Escherichia coli strains for adenylate cyclase function by detecting naturally produced 3′,5′-cyclic adenosine monophosphate (cAMP) in bacterial culture media.

F. Plastibodies

In certain embodiments the subject capture agent is a plastibody. The term “plastibody” refers to polymers imprinted with selected template molecules. See, for example, Bruggemann (2002) Adv Biochem Eng Biotechnol 76:127-63; and Haupt et al. (1998) Trends Biotech. 16:468-475. The plastibody principle is based on molecular imprinting, namely, a recognition site that can be generated by stereoregular display of pendant functional groups that are grafted to the sidechains of a polymeric chain to thereby mimic the binding site of, for example, an antibody.

G. Chimeric Binding Agents Derived from Two Low-Affinity Ligands

Still another strategy for generating suitable capture agents is to link two or more modest-affinity ligands and generate high affinity capture agent. Given the appropriate linker, such chimeric compounds can exhibit affinities that approach the product of the affinities for the two individual ligands for the PET. To illustrate, a collection of compounds is screened at high concentrations for weak interactors of a target PET. The compounds that do not compete with one another are then identified and a library of chimeric compounds is made with linkers of different length. This library is then screened for binding to the PET at much lower concentrations to identify high affinity binders. Such a technique may also be applied to peptides or any other type of modest-affinity PET-binding compound.

H. Labels for Capture Agents

The capture agents of the present invention may be modified to enable detection using techniques known to one of ordinary skill in the art, such as fluorescent, radioactive, chromatic, optical, and other physical or chemical labels, as described herein below.

I. Miscellaneous

In addition, for any given PET, multiple capture agents belonging to each of the above described categories of capture agents may be available. These multiple capture agents may have different properties, such as affinity/avidity/specificity for the PET. Different affinities are useful in covering the wide dynamic ranges of expression which some proteins can exhibit. Depending on specific use, in any given array of capture agents, different types/amounts of capture agents may be present on a single chip/array to achieve optimal overall performance.

In a preferred embodiment, capture agents are raised against PETs that are located on the surface of the protein of interest, e.g., hydrophilic regions. PETs that are located on the surface of the protein of interest may be identified using any of the well known software available in the art. For example, the Naccess program may be used.

Naccess is a program that calculates the accessible area of a molecule from a PDB (Protein Data Bank) format file. It can calculate the atomic and residue accessibilities for both proteins and nucleic acids. Naccess calculates the atomic accessible area when a probe is rolled around the Van der Waal's surface of a macromolecule. Such three-dimensional co-ordinate sets are available from the PDB at the Brookhaven National laboratory. The program uses the Lee & Richards (1971) J. Mol. Biol., 55, 379-400 method, whereby a probe of given radius is rolled around the surface of the molecule, and the path traced out by its center is the accessible surface.

The solvent accessibility method described in Boger, J., Emini, E. A. & Schmidt, A., Surface probability profile-An heuristic approach to the selection of synthetic peptide antigens, Reports on the Sixth International Congress in Immunology (Toronto) 1986 p. 250 also may be used to identify PETs that are located on the surface of the protein of interest. The package MOLMOL (Koradi, R. et al. (1996) J. Mol. Graph. 14:51-55) and Eisenhaber's ASC method (Eisenhaber and Argos (1993) J. Comput. Chem. 14:1272-1280; Eisenhaber et al. (1995) J. Comput. Chem. 16:273-284) may also be used.

In another embodiment, capture agents are raised that are designed to bind with peptides generated by digestion of intact proteins rather than with accessible peptidic surface regions on the proteins. In this embodiment, it is preferred to employ a fragmentation protocol which reproducibly generates all of the PETs in the sample under study.

II. Tools Comprising Capture Agents (Arrays, etc.)

In certain embodiments, to construct arrays, e.g., high-density arrays, of capture agents for efficient screening of complex chemical or biological samples or large numbers of compounds, the capture agents need to be immobilized onto a solid support (e.g., a planar support or a bead). A variety of methods are known in the art for attaching biological molecules to solid supports. See, generally, Affinity Techniques, Enzyme Purification: Part B, Meth. Enz. 34 (ed. W. B. Jakoby and M. Wilchek, Acad. Press, N.Y. 1974) and Immobilized Biochemicals and Affinity Chromatography, Adv. Exp. Med. Biol. 42 (ed. R. Dunlap, Plenum Press, N.Y. 1974). The following are a few considerations when constructing arrays.

A. Formats and Surfaces Consideration

Protein arrays have been designed as a miniaturisation of familiar immunoassay methods such as ELISA and dot blotting, often utilizing fluorescent readout, and facilitated by robotics and high throughput detection systems to enable multiple assays to be carried out in parallel. Common physical supports include glass slides, silicon, microwells, nitrocellulose or PVDF membranes, and magnetic and other microbeads. While microdrops of protein delivered onto planar surfaces are widely used, related alternative architectures include CD centrifugation devices based on developments in microfluidics [Gyros] and specialized chip designs, such as engineered microchannels in a plate [The Living Chip™, Biotrove] and tiny 3D posts on a silicon surface [Zyomyx]. Particles in suspension can also be used as the basis of arrays, providing they are coded for identification; systems include color coding for microbeads [Luminex, Bio-Rad] and semiconductor nanocrystals [QDots™, Quantum Dots], and barcoding for beads [UltraPlex™, Smartbeads] and multimetal microrods [Nanobarcodes™ particles, Surromed]. Beads can also be assembled into planar arrays on semiconductor chips [LEAPS technology, BioArray Solutions].

B. Immobilisation Considerations

The variables in immobilization of proteins such as antibodies include both the coupling reagent and the nature of the surface being coupled to. Ideally, the immobilization method used should be reproducible, applicable to proteins of different properties (size, hydrophilic, hydrophobic), amenable to high throughput and automation, and compatible with retention of fully functional protein activity. Orientation of the surface-bound protein is recognized as an important factor in presenting it to ligand or substrate in an active state; for capture arrays the most efficient binding results are obtained with orientated capture reagents, which generally requires site-specific labeling of the protein.

The properties of a good protein array support surface are that it should be chemically stable before and after the coupling procedures, allow good spot morphology, display minimal nonspecific binding, not contribute a background in detection systems, and be compatible with different detection systems.

Both covalent and noncovalent methods of protein immobilization are used and have various pros and cons. Passive adsorption to surfaces is methodologically simple, but allows little quantitative or orientational control; it may or may not alter the functional properties of the protein, and reproducibility and efficiency are variable. Covalent coupling methods provide a stable linkage, can be applied to a range of proteins and have good reproducibility; however, orientation may be variable, chemical dramatization may alter the function of the protein and requires a stable interactive surface. Biological capture methods utilizing a tag on the protein provide a stable linkage and bind the protein specifically and in reproducible orientation, but the biological reagent must first be immobilized adequately and the array may require special handling and have variable stability.

Several immobilization chemistries and tags have been described for fabrication of protein arrays. Substrates for covalent attachment include glass slides coated with amino- or aldehyde-containing silane reagents [Telechem]. In the Versalinx™ system [Prolinx], reversible covalent coupling is achieved by interaction between the protein derivatized with phenyldiboronic acid, and salicylhydroxamic acid immobilized on the support surface. This also has low background binding and low intrinsic fluorescence and allows the immobilized proteins to retain function. Noncovalent binding of unmodified protein occurs within porous structures such as HydroGel™ [PerkinElmer], based on a 3-dimensional polyacrylamide gel; this substrate is reported to give a particularly low background on glass microarrays, with a high capacity and retention of protein function. Widely used biological capture methods are through biotin/streptavidin or hexahistidine/Ni interactions, having modified the protein appropriately. Biotin may be conjugated to a poly-lysine backbone immobilized on a surface such as titanium dioxide [Zyomyx] or tantalum pentoxide [Zeptosens].

Arenkov et al., for example, have described a way to immobilize proteins while preserving their function by using microfabricated polyacrylamide gel pads to capture proteins, and then accelerating diffusion through the matrix by microelectrophoresis (Arenkov et al. (2000), Anal Biochem 278(2):123-31). The patent literature also describes a number of different methods for attaching biological molecules to solid supports. For example, U.S. Pat. No. 4,282,287 describes a method for modifying a polymer surface through the successive application of multiple layers of biotin, avidin, and extenders. U.S. Pat. No. 4,562,157 describes a technique for attaching biochemical ligands to surfaces by attachment to a photochemically reactive arylazide. U.S. Pat. No. 4,681,870 describes a method for introducing free amino or carboxyl groups onto a silica matrix, in which the groups may subsequently be covalently linked to a protein in the presence of a carbodiimide. In addition, U.S. Pat. No. 4,762,881 describes a method for attaching a polypeptide chain to a solid substrate by incorporating a light-sensitive unnatural amino acid group into the polypeptide chain and exposing the product to low-energy ultraviolet light.

The surface of the support is chosen to possess, or is chemically derivatized to possess, at least one reactive chemical group that can be used for further attachment chemistry. There may be optional flexible adapter molecules interposed between the support and the capture agents. In one embodiment, the capture agents are physically adsorbed onto the support.

In certain embodiments of the invention, a capture agent is immobilized on a support in ways that separate the capture agent's PET binding site region and the region where it is linked to the support. In a preferred embodiment, the capture agent is engineered to form a covalent bond between one of its termini to an adapter molecule on the support. Such a covalent bond may be formed through a Schiff-base linkage, a linkage generated by a Michael addition, or a thioether linkage.

In order to allow attachment by an adapter or directly by a capture agent, the surface of the substrate may require preparation to create suitable reactive groups. Such reactive groups could include simple chemical moieties such as amino, hydroxyl, carboxyl, carboxylate, aldehyde, ester, amide, amine, nitrile, sulfonyl, phosphoryl, or similarly chemically reactive groups. Alternatively, reactive groups may comprise more complex moieties that include, but are not limited to, sulfo-N-hydroxysuccinimide, nitrilotriacetic acid, activated hydroxyl, haloacetyl (e.g., bromoacetyl, iodoacetyl), activated carboxyl, hydrazide, epoxy, aziridine, sulfonylchloride, trifluoromethyldiaziridine, pyridyldisulfide, N-acyl-imidazole, imidazolecarbamate, succinimidylcarbonate, arylazide, anhydride, diazoacetate, benzophenone, isothiocyanate, isocyanate, imidoester, fluorobenzene, biotin and avidin. Techniques of placing such reactive groups on a substrate by mechanical, physical, electrical or chemical means are well known in the art, such as described by U.S. Pat. No. 4,681,870, incorporated herein by reference.

Once the initial preparation of reactive groups on the substrate is completed (if necessary), adapter molecules optionally may be added to the surface of the substrate to make it suitable for further attachment chemistry. Such adapters covalently join the reactive groups already on the substrate and the capture agents to be immobilized, having a backbone of chemical bonds forming a continuous connection between the reactive groups on the substrate and the capture agents, and having a plurality of freely rotating bonds along that backbone. Substrate adapters may be selected from any suitable class of compounds and may comprise polymers or copolymers of organic acids, aldehydes, alcohols, thiols, amines and the like. For example, polymers or copolymers of hydroxy-, amino-, or di-carboxylic acids, such as glycolic acid, lactic acid, sebacic acid, or sarcosine may be employed. Alternatively, polymers or copolymers of saturated or unsaturated hydrocarbons such as ethylene glycol, propylene glycol, saccharides, and the like may be employed. Preferably, the substrate adapter should be of an appropriate length to allow the capture agent, which is to be attached, to interact freely with molecules in a sample solution and to form effective binding. The substrate adapters may be either branched or unbranched, but this and other structural attributes of the adapter should not interfere stereochemically with relevant functions of the capture agents, such as a PET interaction. Protection groups, known to those skilled in the art, may be used to prevent the adapter's end groups from undesired or premature reactions. For instance, U.S. Pat. No. 5,412,087, incorporated herein by reference, describes the use of photo-removable protection groups on a adapter's thiol group.

To preserve the binding affinity of a capture agent, it is preferred that the capture agent be modified so that it binds to the support substrate at a region separate from the region responsible for interacting with it's ligand, i.e., the PET.

Methods of coupling the capture agent to the reactive end groups on the surface of the substrate or on the adapter include reactions that form linkage such as thioether bonds, disulfide bonds, amide bonds, carbamate bonds, urea linkages, ester bonds, carbonate bonds, ether bonds, hydrazone linkages, Schiff-base linkages, and noncovalent linkages mediated by, for example, ionic or hydrophobic interactions. The form of reaction will depend, of course, upon the available reactive groups on both the substrate/adapter and capture agent.

C. Array Fabrication Consideration

Preferably, the immobilized capture agents are arranged in an array on a solid support, such as a silicon-based chip or glass slide. One or more capture agents designed to detect the presence (and optionally the concentration) of a given known protein (one previously recognized as existing) is immobilized at each of a plurality of cells/regions in the array. Thus, a signal at a particular cell/region indicates the presence of a known protein in the sample, and the identity of the protein is revealed by the position of the cell. Alternatively, capture agents for one or a plurality of PET are immobilized on beads, which optionally are labeled to identify their intended target analyte, or are distributed in an array such as a microwell plate.

In one embodiment, the microarray is high density, with a density over about 100, preferably over about 1000, 1500, 2000, 3000, 4000, 5000 and further preferably over about 9000, 10000, 11000, 12000 or 13000 spots per cm², formed by attaching capture agents onto a support surface which has been functionalized to create a high density of reactive groups or which has been functionalized by the addition of a high density of adapters bearing reactive groups. In another embodiment, the microarray comprises a relatively small number of capture agents, e.g., 10 to 50, selected to detect in a sample various combinations of specific proteins which generate patterns probative of disease diagnosis, cell type determination, pathogen identification, etc.

Although the characteristics of the substrate or support may vary depending upon the intended use, the shape, material and surface modification of the substrates must be considered. Although it is preferred that the substrate have at least one surface which is substantially planar or flat, it may also include indentations, protuberances, steps, ridges, terraces and the like and may have any geometric form (e.g., cylindrical, conical, spherical, concave surface, convex surface, string, or a combination of any of these). Suitable substrate materials include, but are not limited to, glasses, ceramics, plastics, metals, alloys, carbon, papers, agarose, silica, quartz, cellulose, polyacrylamide, polyamide, and gelatin, as well as other polymer supports, other solid-material supports, or flexible membrane supports. Polymers that may be used as substrates include, but are not limited to: polystyrene; poly(tetra)fluoroethylene (PTFE); polyvinylidenedifluoride; polycarbonate; polymethylmethacrylate; polyvinylethylene; polyethyleneimine; polyoxymethylene (POM); polyvinylphenol; polylactides; polymethacrylimide (PMI); polyalkenesulfone (PAS); polypropylene; polyethylene; polyhydroxyethylmethacrylate (HEMA); polydimethylsiloxane; polyacrylamide; polyimide; and various block co-polymers. The substrate can also comprise a combination of materials, whether water-permeable or not, in multi-layer configurations. A preferred embodiment of the substrate is a plain 2.5 cm×7.5 cm glass slide with surface Si—OH functionalities.

Array fabrication methods include robotic contact printing, ink-jetting, piezoelectric spotting and photolithography. A number of commercial arrayers are available [e.g. Packard Biosience] as well as manual equipment [V & P Scientific]. Bacterial colonies can be robotically gridded onto PVDF membranes for induction of protein expression in situ.

At the limit of spot size and density are nanoarrays, with spots on the nanometer spatial scale, enabling thousands of reactions to be performed on a single chip less than 1 mm square. BioForce Laboratories have developed nanoarrays with 1521 protein spots in 85 sq microns, equivalent to 25 million spots per sq cm, at the limit for optical detection; their readout methods are fluorescence and atomic force microscopy (AFM).

A microfluidics system for automated sample incubation with arrays on glass slides and washing has been codeveloped by NextGen and PerkinElmer Lifesciences.

For example, capture agent microarrays may be produced by a number of means, including “spotting” wherein small amounts of the reactants are dispensed to particular positions on the surface of the substrate. Methods for spotting include, but are not limited to, microfluidics printing, microstamping (see, e.g., U.S. Pat. No. 5,515,131, U.S. Pat. No. 5,731,152, Martin, B. D. et al. (1998), Langmuir 14: 3971-3975 and Haab, B B et al. (2001) Genome Biol 2 and MacBeath, G. et al. (2000) Science 289: 1760-1763), microcontact printing (see, e.g., PCT Publication WO 96/29629), inkjet head printing (Roda, A. et al. (2000) BioTechniques 28: 492-496, and Silzel, J. W. et al. (1998) Clin Chem 44: 2036-2043), microfluidic direct application (Rowe, C. A. et al. (1999) Anal Chem 71: 433-439 and Bernard, A. et al. (2001), Anal Chem 73: 8-12) and electrospray deposition (Morozov, V. N. et al. (1999) Anal Chem 71: 1415-1420 and Moerman R. et al. (2001) Anal Chem 73: 2183-2189). Generally, the dispensing device includes calibrating means for controlling the amount of sample deposition, and may also include a structure for moving and positioning the sample in relation to the support surface. The volume of fluid to be dispensed per capture agent in an array varies with the intended use of the array, and available equipment. Preferably, a volume formed by one dispensation is less than 100 nL, more preferably less than 10 nL, and most preferably about 1 nL. The size of the resultant spots will vary as well, and in preferred embodiments these spots are less than 20,000 μm in diameter, more preferably less than 2,000 μm in diameter, and most preferably about 150-200 μm in diameter (to yield about 1600 spots per square centimeter). Solutions of blocking agents may be applied to the microarrays to prevent non-specific binding by reactive groups that have not bound to a capture agent. Solutions of bovine serum albumin (BSA), casein, or nonfat milk, for example, may be used as blocking agents to reduce background binding in subsequent assays.

In preferred embodiments, high-precision, contact-printing robots are used to pick up small volumes of dissolved capture agents from the wells of a microtiter plate and to repetitively deliver approximately 1 nL of the solutions to defined locations on the surfaces of substrates, such as chemically-derivatized glass microscope slides. Examples of such robots include the GMS 417 Arrayer, commercially available from Affymetrix of Santa Clara, Calif., and a split pin arrayer constructed according to instructions downloadable from the Brown lab website at http://cmgm.stanford.edu/pbrown. This results in the formation of microscopic spots of compounds on the slides. It will be appreciated by one of ordinary skill in the art, however, that the current invention is not limited to the delivery of 1 nL volumes of solution, to the use of particular robotic devices, or to the use of chemically derivatized glass slides, and that alternative means of delivery can be used that are capable of delivering picoliter or smaller volumes. Hence, in addition to a high precision array robot, other means for delivering the compounds can be used, including, but not limited to, ink jet printers, piezoelectric printers, and small volume pipetting robots.

In one embodiment, the compositions, e.g., microarrays or beads, comprising the capture agents of the present invention may also comprise other components, e.g., molecules that recognize and bind specific peptides, metabolites, drugs or drug candidates, RNA, DNA, lipids, and the like. Thus, an array of capture agents only some of which bind a PET can comprise an embodiment of the invention.

As an alternative to planar microarrays, bead-based assays combined with fluorescence-activated cell sorting (FACS) have been developed to perform multiplexed immunoassays. Fluorescence-activated cell sorting has been routinely used in diagnostics for more than 20 years. Using mAbs, cell surface markers are identified on normal and neoplastic cell populations enabling the classification of various forms of leukemia or disease monitoring (recently reviewed by Herzenberg et al. Immunol Today 21 (2000), pp. 383-390).

Bead-based assay systems employ microspheres as solid support for the capture molecules instead of a planar substrate, which is conventionally used for microarray assays. In each individual immunoassay, the capture agent is coupled to a distinct type of microsphere. The reaction takes place on the surface of the microspheres. The individual microspheres are color-coded by a uniform and distinct mixture of red and orange fluorescent dyes. After coupling to the appropriate capture molecule, the different color-coded bead sets can be pooled and the immunoassay is performed in a single reaction vial. Product formation of the PET targets with their respective capture agents on the different bead types can be detected with a fluorescence-based reporter system. The signal intensities are measured in a flow cytometer, which is able to quantify the amount of captured targets on each individual bead. Each bead type and thus each immobilized target is identified using the color code measured by a second fluorescence signal. This allows the multiplexed quantification of multiple targets from a single sample. Sensitivity, reliability and accuracy are similar to those observed with standard microtiter ELISA procedures. Color-coded microspheres can be used to perform up to a hundred different assay types simultaneously (LabMAP system, Laboratory Muliple Analyte Profiling, Luminex, Austin, Tex., USA). For example, microsphere-based systems have been used to simultaneously quantify cytokines or autoantibodies from biological samples (Carson and Vignali, J Immunol Methods 227 (1999), pp. 41-52; Chen et al., Clin Chem 45 (1999), pp. 1693-1694; Fulton et al., Clin Chem 43 (1997), pp. 1749-1756). Bellisario et al. (Early Hum Dev 64 (2001), pp. 21-25) have used this technology to simultaneously measure antibodies to three HIV-1 antigens from newborn dried blood-spot specimens.

Bead-based systems have several advantages. As the capture molecules are coupled to distinct microspheres, each individual coupling event can be perfectly analyzed. Thus, only quality-controlled beads can be pooled for multiplexed immunoassays. Furthermore, if an additional parameter has to be included into the assay, one must only add a new type of loaded bead. No washing steps are required when performing the assay. The sample is incubated with the different bead types together with fluorescently labeled detection antibodies. After formation of the sandwich immuno-complex, only the fluorophores that are definitely bound to the surface of the microspheres are counted in the flow cytometer.

D. Related Non-Array Formats

An alternative to an array of capture agents is one made through the so-called “molecular imprinting” technology, in which peptides (e.g. selected PETs) are used as templates to generate structurally complementary, sequence-specific cavities in a polymerisable matrix; the cavities can then specifically capture (digested) proteins which have the appropriate primary amino acid sequence [ProteinPrint™, Aspira Biosystems]. To illustrate, a chosen PET can be synthesized, and a universal matrix of polymerizable monomers is allowed to self assemble around the peptide and crosslinked into place. The PET, or template, is then removed, leaving behind a cavity complementary in shape and functionality. The cavities can be formed on a film, discrete sites of an array or the surface of beads. When a sample of fragmented proteins is exposed to the capture agent, the polymer will selectively retain the target protein containing the PET and exclude all others. After the washing, only the bound PET-containing peptides remain. Common staining and tagging procedures, or any of the non-labeling techniques described below can be used to detect expression levels and/or post translational modifications. See, for example, WO 01/61354 A1 and WO 01/61355 A1.

Alternatively, the captured peptides can be eluted for further analysis such as mass spectrometry analysis. Although several well-established chemical methods for the sequencing of peptides, polypeptides and proteins are known (for example, the Edman degradation), mass spectrometric methods are becoming increasingly important in view of their speed and ease of use. Mass spectrometric methods have been developed to the point at which they are capable of sequencing peptides in a mixture even without any prior chemical purification or separation, typically using electrospray ionization and tandem mass spectrometry (MS/MS). For example, see Yates III (J. Mass Spectrom, 1998 vol. 33 pp. 1-19), Papayannopoulos (Mass Spectrom. Rev. 1995, vol. 14 pp. 49-73), and Yates III, McCormack, and Eng (Anal. Chem. 1996 vol. 68 (17) pp. 534A-540A). Thus, in a typical MS/MS sequencing experiment, molecular ions of a particular peptide are selected by the first mass analyzer and fragmented by collisions with neutral gas molecules in a collision cell. The second mass analyzer is then used to record the fragment ion spectrum that generally contains enough information to allow at least a partial, and often the complete, sequence to be determined. See, for example, U.S. Pat. Nos. 6,489,608, 5,470,753, 5,246,865, all incorporated hereion by reference, and related applications/patents.

Another methodology which can be used diagnostically and in expression profiling is the ProteinChip® array [Ciphergen], in which solid phase chromatographic surfaces bind proteins with similar characteristics of charge or hydrophobicity from mixtures such as plasma or tumor extracts, and SELDI-TOF mass spectrometry is used to detection the retained proteins. The ProteinChip® is credited with the ability to identify novel disease markers. However, this technology differs from the protein arrays under discussion here since, in general, it does not involve immobilization of individual proteins for detection of specific ligand interactions.

E. Single Assay Format

PET-specific affinity capture agents can also be used in a single assay format. For example, such agents can be used to develop a better assay for detecting circulating agents, such as PSA, by providing increased sensitivity, dynamic range and/or recovery rate. For instance, the single assays can have functional performance characteristics which exceed traditional ELISA and other immunoassays, such as one or more of the following: a regression coefficient (R2) of 0.95 or greater for a reference standard, e.g., a comparable control sample, more preferably an R2 greater than 0.97, 0.99 or even 0.995; a recovery rate of at least 50 percent, and more preferably at least 60, 75, 80 or even 90 percent; a positive predictive value for occurrence of the protein in a sample of at least 90 percent, more preferably at least 95, 98 or even 99 percent; a diagnostic sensitivity (DSN) for occurrence of the protein in a sample of 99 percent or higher, more preferably at least 99.5 or even 99.8 percent; a diagnostic specificity (DSP) for occurrence of the protein in a sample of 99 percent or higher, more preferably at least 99.5 or even 99.8 percent.

III. Methods of Detecting Binding Events

The capture agents of the invention, as well as compositions, e.g., microarrays or beads, comprising these capture agents have a wide range of applications in the health care industry, e.g., in therapy, in clinical diagnostics, in in vivo imaging or in drug discovery. The capture agents of the present invention also have industrial and environmental applications, e.g., in environmental diagnostics, industrial diagnostics, food safety, toxicology, catalysis of reactions, or high-throughput screening; as well as applications in the agricultural industry and in basic research, e.g., protein sequencing.

The capture agents of the present invention are a powerful analytical tool that enables a user to detect a specific protein, or group of proteins of interest present within complex samples. In addition, the invention allow for efficient and rapid analysis of samples; sample conservation and direct sample comparison. The invention enables “multi-parametric” analysis of protein samples. As used herein, a “multi-parametric” analysis of a protein sample is intended to include an analysis of a protein sample based on a plurality of parameters. For example, a protein sample may be contacted with a plurality of PETs, each of the PETs being able to detect a different protein within the sample. Based on the combination and, preferably the relative concentration, of the proteins detected in the sample the skilled artisan would be able to determine the identity of a sample, diagnose a disease or pre-disposition to a disease, or determine the stage of a disease

The capture agents of the present invention may be used in any method suitable for detection of a protein or a polypeptide, such as, for example, in immunoprecipitations, immunocytochemistry, Western Blots or nuclear magnetic resonance spectroscopy (NMR).

To detect the presence of a protein that interacts with a capture agent, a variety of art known methods may be used. The protein to be detected may be labeled with a detectable label, and the amount of bound label directly measured. The term “label” is used herein in a broad sense to refer to agents that are capable of providing a detectable signal, either directly or through interaction with one or more additional members of a signal producing system. Labels that are directly detectable and may find use in the present invention include, for example, fluorescent labels such as fluorescein, rhodamine, BODIPY, cyanine dyes (e.g. from Amersham Pharmacia), Alexa dyes (e.g. from Molecular Probes, Inc.), fluorescent dye phosphoramidites, beads, chemilumninescent compounds, colloidal particles, and the like. Suitable fluorescent dyes are known in the art, including fluoresceinisothiocyanate (FITC); rhodamine and rhodamine derivatives; Texas Red; phycoerythrin; allophycocyanin; 6-carboxyfluorescein (6-FAM); 2′,7′-dimethoxy-41,51-dichloro carboxyfluorescein (JOE); 6-carboxy-X-rhodamine (ROX); 6-carboxy-21,41,71,4,7-hexachlorofluorescein (HEX); 5-carboxyfluorescein (5-FAM); N,N,N1,N′-tetramethyl carboxyrhodamine (TAMRA); sulfonated rhodamine; Cy3; Cy5, etc. Radioactive isotopes, such as ³⁵S, ³²P, ³H, ¹²⁵I, etc., and the like can also be used for labeling. In addition, labels may also include near-infrared dyes (Wang et al., Anal. Chem., 72:5907-5917 (2000), upconverting phosphors (Hampl et al., Anal. Biochem., 288:176-187 (2001), DNA dendrimers (Stears et al., Physiol. Genomics 3: 93-99 (2000), quantum dots (Bruchez et al., Science 281:2013-2016 (1998), latex beads (Okana et al., Anal. Biochem. 202:120-125 (1992), selenium particles (Stimpson et al., Proc. Natl. Acad. Sci. 92:6379-6383 (1995), and europium nanoparticles (Harma et al., Clin. Chem. 47:561-568 (2001). The label is one that preferably does not provide a variable signal, but instead provides a constant and reproducible signal over a given period of time.

A very useful labeling agent is water-soluble quantum dots, or so-called “functionalized nanocrystals” or “semiconductor nanocrystals” as described in U.S. Pat. No. 6,114,038. Generally, quantum dots can be prepared which result in relative monodispersity (e.g., the diameter of the core varying approximately less than 10% between quantum dots in the preparation), as has been described previously (Bawendi et al., 1993, J. Am. Chem. Soc. 115:8706). Examples of quantum dots are known in the art to have a core selected from the group consisting of CdSe, CdS, and CdTe (collectively referred to as “CdX”)(see, e.g., Norris et al., 1996, Physical Review B. 53:16338-16346; Nirmal et al., 1996, Nature 383:802-804; Empedocles et al., 1996, Physical Review Letters 77:3873-3876; Murray et al., 1996, Science 270: 1355-1338; Effros et al., 1996, Physical Review B. 54:4843-4856; Sacra et al., 1996, J. Chem. Phys. 103:5236-5245; Murakoshi et al., 1998, J. Colloid Interface Sci. 203:225-228; Optical Materials and Engineering News, 1995, Vol. 5, No. 12; and Murray et al., 1993, J. Am. Chem. Soc. 115:8706-8714; the disclosures of which are hereby incorporated by reference).

CdX quantum dots have been passivated with an inorganic coating (“shell”) uniformly deposited thereon. Passivating the surface of the core quantum dot can result in an increase in the quantum yield of the luminescence emission, depending on the nature of the inorganic coating. The shell which is used to passivate the quantum dot is preferably comprised of YZ wherein Y is Cd or Zn, and Z is S, or Se. Quantum dots having a CdX core and a YZ shell have been described in the art (see, e.g., Danek et al., 1996, Chem. Mater. 8:173-179; Dabbousi et al., 1997, J. Phys. Chem. B 101:9463; Rodriguez-Viejo et al., 1997, Appl. Phys. Lett. 70:2132-2134; Peng et al., 1997, J. Am. Chem. Soc. 119:7019-7029; 1996, Phys. Review B. 53:16338-16346; the disclosures of which are hereby incorporated by reference). However, the above described quantum dots, passivated using an inorganic shell, have only been soluble in organic, non-polar (or weakly polar) solvents. To make quantum dots useful in biological applications, it is desirable that the quantum dots are water-soluble. “Water-soluble” is used herein to mean sufficiently soluble or suspendable in an aqueous-based solution, such as in water or water-based solutions or buffer solutions, including those used in biological or molecular detection systems as known by those skilled in the art.

U.S. Pat. No. 6,114,038 provides a composition comprising functionalized nanocrystals for use in non-isotopic detection systems. The composition comprises quantum dots (capped with a layer of a capping compound) that are water-soluble and functionalized by operably linking, in a successive manner, one or more additional compounds. In a preferred embodiment, the one or more additional compounds form successive layers over the nanocrystal. More particularly, the functionalized nanocrystals comprise quantum dots capped with the capping compound, and have at least a diaminocarboxylic acid which is operatively linked to the capping compound. Thus, the functionalized nanocrystals may have a first layer comprising the capping compound, and a second layer comprising a diaminocarboxylic acid; and may further comprise one or more successive layers including a layer of amino acid, a layer of affinity ligand, or multiple layers comprising a combination thereof. The composition comprises a class of quantum dots that can be excited with a single wavelength of light resulting in detectable luminescence emissions of high quantum yield and with discrete luminescence peaks. Such functionalized nanocrystal may be used to label capture agents of the instant invention for their use in the detection and/or quantitation of the binding events.

U.S. Pat. No. 6,326,144 describes quantum dots (QDs) having a characteristic spectral emission, which is tunable to a desired energy by selection of the particle size of the quantum dot. For example, a 2 nanometer quantum dot emits green light, while a 5 nanometer quantum dot emits red light. The emission spectra of quantum dots have linewidths as narrow as 25-30 nm depending on the size heterogeneity of the sample, and lineshapes that are symmetric, gaussian or nearly gaussian with an absence of a tailing region. The combination of tunability, narrow linewidths, and symmetric emission spectra without a tailing region provides for high resolution of multiply-sized quantum dots within a system and enables researchers to examine simultaneously a variety of biological moieties tagged with QDs. In addition, the range of excitation wavelengths of the nanocrystal quantum dots is broad and can be higher in energy than the emission wavelengths of all available quantum dots. Consequently, this allows the simultaneous excitation of all quantum dots in a system with a single light source, usually in the ultraviolet or blue region of the spectrum. QDs are also more robust than conventional organic fluorescent dyes and are more resistant to photobleaching than the organic dyes. The robustness of the QD also alleviates the problem of contamination of the degradation products of the organic dyes in the system being examined. These QDs can be used for labeling capture agents of protein, nucleic acid, and other biological molecules in nature. Cadmium Selenide quantum dot nanocrystals are available from Quantum Dot Corporation of Hayward, Calif.

Alternatively, the sample to be tested is not labeled, but a second stage labeled reagent is added in order to detect the presence or quantitate the amount of protein in the sample. Such “sandwich based” methods of detection have the disadvantage that two capture agents must be developed for each protein, one to capture the PET and one to label it once captured. Such methods have the advantage that they are characterized by an inherently improved signal to noise ratio as they exploit two binding reactions at different points on a peptide, thus the presence and/or concentration of the protein can be measured with more accuracy and precision because of the increased signal to noise ratio.

In yet another embodiment, the subject capture array can be a “virtual arrays”. For example, a virtual array can be generated in which antibodies or other capture agents are immobilized on beads whose identity, with respect to the particular PET it is specific for as a consequence to the associated capture agent, is encoded by a particular ratio of two or more covalently attached dyes. Mixtures of encoded PET-beads are added to a sample, resulting in capture of the PET entities recognized by the immobilized capture agents.

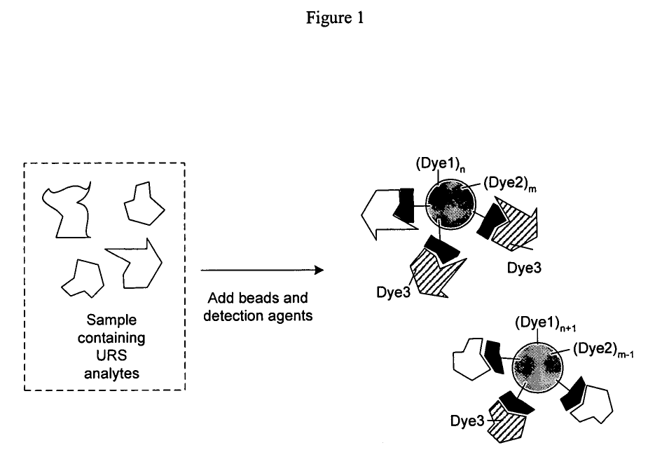

To quantitate the captured species, a sandwich assay with fluorescently labeled antibodies that bind the captured PET, or a competitive binding assay with a fluorescently labeled ligand for the capture agent, are added to the mix. In one embodiment, the labeled ligand is a labeled PET that competes with the analyte PET for binding to the capture agent. The beads are then introduced into an instrument, such as a flow cytometer, that reads the intensity of the various fluorescence signals on each bead, and the identity of the bead can be determined by measuring the ratio of the dyes (FIG. 1). This technology is relatively fast and efficient, and can be adapted by researchers to monitor almost any set of PET of interest.

In another embodiment, an array of capture agents are embedded in a matrix suitable for ionization (such as described in Fung et al. (2001) Curr. Opin. Biotechnol. 12:65-69). After application of the sample and removal of unbound molecules (by washing), the retained PET proteins are analyzed by mass spectrometry. In some instances, further proteolytic digestion of the bound species with trypsin may be required before ionization, particularly if electrospray is the means for ionizing the peptides.

All the above named reagents may be used to label the capture agents. Preferably, the capture agent to be labeled is combined with an activated dye that reacts with a group present on the protein to be detected, e.g., amine groups, thiol groups, or aldehyde groups.

The label may also be a covalently bound enzyme capable of providing a detectable product signal after addition of suitable substrate. Examples of suitable enzymes for use in the present invention include horseradish peroxidase, alkaline phosphatase, malate dehydrogenase and the like.

Enzyme-Linked Immunosorbent Assay (ELISA) may also be used for detection of a protein that interacts with a capture agent. In an ELISA, the indicator molecule is covalently coupled to an enzyme and may be quantified by determining with a spectrophotometer the initial rate at which the enzyme converts a clear substrate to a correlated product. Methods for performing ELISA are well known in the art and described in, for example, Perlmann, H. and Perlmann, P. (1994). Enzyme-Linked Immunosorbent Assay. In: Cell Biology: A Laboratory Handbook. San Diego, Calif., Academic Press, Inc., 322-328; Crowther, J. R. (1995). Methods in Molecular Biology, Vol. 42-ELISA: Theory and Practice. Humana Press, Totowa, N.J.; and Harlow, E. and Lane, D. (1988). Antibodies: A Laboratory Manual. Cold Spring Harbor Laboratory Press, Cold Spring Harbor, N.Y., 553-612, the contents of each of which are incorporated by reference. Sandwich (capture) ELISA may also be used to detect a protein that interacts with two capture agents. The two capture agents may be able to specifically interact with two PETs that are present on the same peptide (e.g., the peptide which has been generated by fragmentation of the sample of interest, as described above). Alternatively, the two capture agents may be able to specifically interact with one PET and one non-unique amino acid sequence, both present on the same peptide (e.g., the peptide which has been generated by fragmentation of the sample of interest, as described above). Sandwich ELISAs for the quantitation of proteins of interest are especially valuable when the concentration of the protein in the sample is low and/or the protein of interest is present in a sample that contains high concentrations of contaminating proteins.

A fully-automated, microarray-based approach for high-throughput, ELISAs was described by Mendoza et al. (BioTechniques 27:778-780, 782-786, 788, 1999). This system consisted of an optically flat glass plate with 96 wells separated by a Teflon mask. More than a hundred capture molecules were immobilized in each well. Sample incubation, washing and fluorescence-based detection were performed with an automated liquid pipettor. The microarrays were quantitatively imaged with a scanning charge-coupled device (CCD) detector. Thus, the feasibility of multiplex detection of arrayed antigens in a high-throughput fashion using marker antigens could be successfully demonstrated. In addition, Silzel et al. (Clin Chem 44 pp. 2036-2043, 1998) could demonstrate that multiple IgG subclasses can be detected simultaneously using microarray technology. Wiese et al. (Clin Chem 47 pp. 1451-1457, 2001) were able to measure prostate-specific antigen (PSA), -(1)-antichymotrypsin-bound PSA and interleukin-6 in a microarray format. Arenkov et al. (supra) carried out microarray sandwich immunoassays and direct antigen or antibody detection experiments using a modified polyacrylamide gel as substrate for immobilized capture molecules.

Most of the microarray assay formats described in the art rely on chemiluminescence- or fluorescence-based detection methods. A further improvement with regard to sensitivity involves the application of fluorescent labels and waveguide technology. A fluorescence-based array immunosensor was developed by Rowe et al. (Anal Chem 71 (1999), pp. 433-439; and Biosens Bioelectron 15 (2000), pp. 579-589) and applied for the simultaneous detection of clinical analytes using the sandwich immunoassay format. Biotinylated capture antibodies were immobilized on avidin-coated waveguides using a flow-chamber module system. Discrete regions of capture molecules were vertically arranged on the surface of the waveguide. Samples of interest were incubated to allow the targets to bind to their capture molecules. Captured targets were then visualized with appropriate fluorescently labeled detection molecules. This array immunosensor was shown to be appropriate for the detection and measurement of targets at physiologically relevant concentrations in a variety of clinical samples.

A further increase in the sensitivity using waveguide technology was achieved with the development of the planar waveguide technology (Duveneck et al., Sens Actuators B B38 (1997), pp. 88-95). Thin-film waveguides are generated from a high-refractive material such as Ta₂O₅ that is deposited on a transparent substrate. Laser light of desired wavelength is coupled to the planar waveguide by means of diffractive grating. The light propagates in the planar waveguide and an area of more than a square centimeter can be homogeneously illuminated. At the surface, the propagating light generates a so-called evanescent field. This extends into the solution and activates only fluorophores that are bound to the surface. Fluorophores in the surrounding solution are not excited. Close to the surface, the excitation field intensities can be a hundred times higher than those achieved with standard confocal excitation. A CCD camera is used to identify signals simultaneously across the entire area of the planar waveguide. Thus, the immobilization of the capture molecules in a microarray format on the planar waveguide allows the performance of highly sensitive miniaturized and parallelized immunoassays. This system was successfully employed to detect interleukin-6 at concentrations as low as 40 fM and has the additional advantage that the assay can be performed without washing steps that are usually required to remove unbound detection molecules (Weinberger et al., Pharmacogenomics 1 (2000), pp. 395-416).

Alternative strategies pursued to increase sensitivity are based on signal amplification procedures. For example, immunoRCA (immuno rolling circle amplification) involves an oligonucleotide primer that is covalently attached to a detection molecule (such as a second capture agent in a sandwich-type assay format). Using circular DNA as template, which is complementary to the attached oligonucleotide, DNA polymerase will extend the attached oligonucleotide and generate a long DNA molecule consisting of hundreds of copies of the circular DNA, which remains attached to the detection molecule. The incorporation of thousands of fluorescently labeled nucleotides will generate a strong signal. Schweitzer et al. (Proc Natl Acad Sci USA 97 (2000), pp. 10113-10119) have evaluated this detection technology for use in microarray-based assays. Sandwich immunoassays for huIgE and prostate-specific antigens were performed in a microarray format. The antigens could be detected at femtomolar concentrations and it was possible to score single, specifically captured antigens by counting discrete fluorescent signals that arose from the individual antibody-antigen complexes. The authors demonstrated that immunoassays employing rolling circle DNA amplification are a versatile platform for the ultra-sensitive detection of antigens and thus are well suited for use in protein microarray technology.

A novel technology for protein detection, proximity ligation, has recently been developed, along with improved methods for in situ synthesis of DNA microarrays. Proximity ligation may be another amplification strategy that can be employed with anti-PET antibodies. Proximity ligation enables a specific and quantitative transformation of proteins present in a sample into nucleic acid sequences. As pairs of so-called proximity probes bind the individual target molecules at distinct sites (say two adjacent epitopes on the same target molecule), these proximity probes are brought in close proximity. The probes consist of a protein specific binding part coupled to an oligonucleotide with either a free 3′- or 5′-end capable of hybridizing to a common connector oligonucleotide. When the probes are in proximity, promoted by target binding, the polynucleotide strands can be joined by enzymatic ligation. The nucleic acid sequence that is formed can then be amplified and quantitatively detected in a real-time monitored polymerase chain reaction or any type of polynucleotide amplification method (such as rolling circle amplification, etc.). In certain embodiments, the common connector oligonucleotide may be omitted, and the ends of the oligonucleotides on the proximity probes may be directly ligated by, for example, T4 DNA ligase. This convenient assay is simple to perform and allows highly sensitive protein detection. It also eliminates or significantly reduces background issue associated with the immuno-PCR method (Sano et al., Chemtech January 1995, pp 24-30), where non-specifically bound oligonucleotides may also be accidentally amplified by the very sensitive PCR method. See WO 97/00446, WO 01/61037 and WO 03/044231, entire contents of which are all incorporated herein by reference.

In certain embodiments, immuno-PCR method such as those described in Sano et al., Chemtech January 1995, pp 24-30 (incorporated herein by reference) may be used to detect any capture agents (e.g. Ab) that specifically bind the immobilized target analytes.

Radioimmunoassays (RIA) may also be used for detection of a protein that interacts with a capture agent. In a RIA, the indicator molecule is labeled with a radioisotope and it may be quantified by counting radioactive decay events in a scintillation counter. Methods for performing direct or competitive RIA are well known in the art and described in, for example, Cell Biology: A Laboratory Handbook. San Diego, Calif., Academic Press, Inc., the contents of which are incorporated herein by reference.

Other immunoassays commonly used to quantitate the levels of proteins in cell samples, and are well-known in the art, can be adapted for use in the instant invention. The invention is not limited to a particular assay procedure, and therefore is intended to include both homogeneous and heterogeneous procedures. Exemplary other immunoassays which can be conducted according to the invention include fluorescence polarization immunoassay (FPIA), fluorescence immunoassay (FIA), enzyme immunoassay (EIA), nephelometric inhibition immunoassay (NIA). An indicator moiety, or label group, can be attached to the subject antibodies and is selected so as to meet the needs of various uses of the method which are often dictated by the availability of assay equipment and compatible immunoassay procedures. General techniques to be used in performing the various immunoassays noted above are known to those of ordinary skill in the art. In one embodiment, the determination of protein level in a biological sample may be performed by a microarray analysis (protein chip).

In several other embodiments, detection of the presence of a protein that interacts with a capture agent may be achieved without labeling. For example, determining the ability of a protein to bind to a capture agent can be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore).

In another embodiment, a biosensor with a special diffractive grating surface may be used to detect/quantitate binding between non-labeled PET-containing peptides in a treated (digested) biological sample and immobilized capture agents at the surface of the biosensor. Details of the technology is described in more detail in B. Cunningham, P. Li, B. Lin, J. Pepper, “Colorimetric resonant reflection as a direct biochemical assay technique,” Sensors and Actuators B, Volume 81, p. 316-328, Jan. 5 2002, and in PCT No. WO 02/061429 A2 and U.S. 2003/0032039. Briefly, a guided mode resonant phenomenon is used to produce an optical structure that, when illuminated with collimated white light, is designed to reflect only a single wavelength (color). When molecules are attached to the surface of the biosensor, the reflected wavelength (color) is shifted due to the change of the optical path of light that is coupled into the grating. By linking receptor molecules to the grating surface, complementary binding molecules can be detected/quantitated without the use of any kind of fluorescent probe or particle label. The spectral shifts may be analyzed to determine the expression data provided, and to indicate the presence or absence of a particular indication.

The biosensor typically comprises: a two-dimensional grating comprised of a material having a high refractive index, a substrate layer that supports the two-dimensional grating, and one or more detection probes immobilized on the surface of the two-dimensional grating opposite of the substrate layer. When the biosensor is illuminated a resonant grating effect is produced on the reflected radiation spectrum. The depth and period of the two-dimensional grating are less than the wavelength of the resonant grating effect.

A narrow band of optical wavelengths can be reflected from the biosensor when it is illuminated with a broad band of optical wavelengths. The substrate can comprise glass, plastic or epoxy. The two-dimensional grating can comprise a material selected from the group consisting of zinc sulfide, titanium dioxide, tantalum oxide, and silicon nitride.

The substrate and two-dimensional grating can optionally comprise a single unit. The surface of the single unit comprising the two-dimensional grating is coated with a material having a high refractive index, and the one or more detection probes are immobilized on the surface of the material having a high refractive index opposite of the single unit. The single unit can be comprised of a material selected from the group consisting of glass, plastic, and epoxy.

The biosensor can optionally comprise a cover layer on the surface of the two-dimensional grating opposite of the substrate layer. The one or more detection probes are immobilized on the surface of the cover layer opposite of the two-dimensional grating. The cover layer can comprise a material that has a lower refractive index than the high refractive index material of the two-dimensional grating. For example, a cover layer can comprise glass, epoxy, and plastic.

A two-dimensional grating can be comprised of a repeating pattern of shapes selected from the group consisting of lines, squares, circles, ellipses, triangles, trapezoids, sinusoidal waves, ovals, rectangles, and hexagons. The repeating pattern of shapes can be arranged in a linear grid, i.e., a grid of parallel lines, a rectangular grid, or a hexagonal grid. The two-dimensional grating can have a period of about 0.01 microns to about 1 micron and a depth of about 0.01 microns to about 1 micron.

To illustrate, biochemical interactions occurring on a surface of a calorimetric resonant optical biosensor embedded into a surface of a microarray slide, microtiter plate or other device, can be directly detected and measured on the sensor's surface without the use of fluorescent tags or calorimetric labels. The sensor surface contains an optical structure that, when illuminated with collimated white light, is designed to reflect only a narrow band of wavelengths (color). The narrow wavelength is described as a wavelength “peak.” The “peak wavelength value” (PWV) changes when biological material is deposited or removed from the sensor surface, such as when binding occurs. Such binding-induced change of PWV can be measured using a measurement instrument disclosed in U.S. 2003/0032039.

In one embodiment, the instrument illuminates the biosensor surface by directing a collimated white light on to the sensor structure. The illuminated light may take the form of a spot of collimated light. Alternatively, the light is generated in the form of a fan beam. The instrument collects light reflected from the illuminated biosensor surface. The instrument may gather this reflected light from multiple locations on the biosensor surface simultaneously. The instrument can include a plurality of illumination probes that direct the light to a discrete number of positions across the biosensor surface. The instrument measures the Peak Wavelength Values (PWVs) of separate locations within the biosensor-embedded microtiter plate using a spectrometer. In one embodiment, the spectrometer is a single-point spectrometer. Alternatively, an imaging spectrometer is used. The spectrometer can produce a PWV image map of the sensor surface. In one embodiment, the measuring instrument spatially resolves PWV images with less than 200 micron resolution.

In one embodiment, a subwavelength structured surface (SWS) may be used to create a sharp optical resonant reflection at a particular wavelength that can be used to track with high sensitivity the interaction of biological materials, such as specific binding substances or binding partners or both. A colormetric resonant diffractive grating surface acts as a surface binding platform for specific binding substances (such as immobilized capture agents of the instant invention). SWS is an unconventional type of diffractive optic that can mimic the effect of thin-film coatings. (Peng & Morris, “Resonant scattering from two-dimensional gratings,” J. Opt. Soc. Am. A, Vol. 13, No. 5, p. 993, May; Magnusson, & Wang, “New principle for optical filters,” Appl. Phys. Lett., 61, No. 9, p. 1022, August, 1992; Peng & Morris, “Experimental demonstration of resonant anomalies in diffraction from two-dimensional gratings,” Optics Letters, Vol. 21, No. 8, p. 549, April, 1996). A SWS structure contains a surface-relief, two-dimensional grating in which the grating period is small compared to the wavelength of incident light so that no diffractive orders other than the reflected and transmitted zeroth orders are allowed to propagate. A SWS surface narrowband filter can comprise a two-dimensional grating sandwiched between a substrate layer and a cover layer that fills the grating grooves. Optionally, a cover layer is not used. When the effective index of refraction of the grating region is greater than the substrate or the cover layer, a waveguide is created. When a filter is designed accordingly, incident light passes into the waveguide region. A two-dimensional grating structure selectively couples light at a narrow band of wavelengths into the waveguide. The light propagates only a short distance (on the order of 10-100 micrometers), undergoes scattering, and couples with the forward- and backward-propagating zeroth-order light. This sensitive coupling condition can produce a resonant grating effect on the reflected radiation spectrum, resulting in a narrow band of reflected or transmitted wavelengths (colors). The depth and period of the two-dimensional grating are less than the wavelength of the resonant grating effect.

The reflected or transmitted color of this structure can be modulated by the addition of molecules such as capture agents or their PET-containing binding partners or both, to the upper surface of the cover layer or the two-dimensional grating surface. The added molecules increase the optical path length of incident radiation through the structure, and thus modify the wavelength (color) at which maximum reflectance or transmittance will occur. Thus in one embodiment, a biosensor, when illuminated with white light, is designed to reflect only a single wavelength. When specific binding substances are attached to the surface of the biosensor, the reflected wavelength (color) is shifted due to the change of the optical path of light that is coupled into the grating. By linking specific binding substances to a biosensor surface, complementary binding partner molecules can be detected without the use of any kind of fluorescent probe or particle label. The detection technique is capable of resolving changes of, for example, about 0.1 nm thickness of protein binding, and can be performed with the biosensor surface either immersed in fluid or dried. This PWV change can be detected by a detection system consists of, for example, a light source that illuminates a small spot of a biosensor at normal incidence through, for example, a fiber optic probe. A spectrometer collects the reflected light through, for example, a second fiber optic probe also at normal incidence. Because no physical contact occurs between the excitation/detection system and the biosensor surface, no special coupling prisms are required. The biosensor can, therefore, be adapted to a commonly used assay platform including, for example, microtiter plates and microarray slides. A spectrometer reading can be performed in several milliseconds, thus it is possible to efficiently measure a large number of molecular interactions taking place in parallel upon a biosensor surface, and to monitor reaction kinetics in real time.

Various embodiments, variations of the biosensor described above can be found in U.S. 2003/0032039, incorporated herein by reference in its entirety.

One or more specific capture agents may be immobilized on the two-dimensional grating or cover layer, if present. Immobilization may occur by any of the above described methods. Suitable capture agents can be, for example, a nucleic acid, polypeptide, antigen, polyclonal antibody, monoclonal antibody, single chain antibody (scFv), F(ab) fragment, F(ab′)2 fragment, Fv fragment, small organic molecule, even cell, virus, or bacteria. A biological sample can be obtained and/or deribed from, for example, blood, plasma, serum, gastrointestinal secretions, homogenates of tissues or tumors, synovial fluid, feces, saliva, sputum, cyst fluid, amniotic fluid, cerebrospinal fluid, peritoneal fluid, lung lavage fluid, semen, lymphatic fluid, tears, or prostatitc fluid. Preferably, one or more specific capture agents are arranged in a microarray of distinct locations on a biosensor. A microarray of capture agents comprises one or more specific capture agents on a surface of a biosensor such that a biosensor surface contains a plurality of distinct locations, each with a different capture agent or with a different amount of a specific capture agent. For example, an array can comprise 1, 10, 100, 1,000, 10,000, or 100,000 distinct locations. A biosensor surface with a large number of distinct locations is called a microarray because one or more specific capture agents are typically laid out in a regular grid pattern in x-y coordinates. However, a microarray can comprise one or more specific capture agents laid out in a regular or irregular pattern.

A microarray spot can range from about 50 to about 500 microns in diameter. Alternatively, a microarray spot can range from about 150 to about 200 microns in diameter. One or more specific capture agents can be bound to their specific PET-containing binding partners.

In one biosensor embodiment, a microarray on a biosensor is created by placing microdroplets of one or more specific capture agents onto, for example, an x-y grid of locations on a two-dimensional grating or cover layer surface. When the biosensor is exposed to a test sample comprising one or more PET binding partners, the binding partners will be preferentially attracted to distinct locations on the microarray that comprise capture agents that have high affinity for the PET binding partners. Some of the distinct locations will gather binding partners onto their surface, while other locations will not. Thus a specific capture agent specifically binds to its PET binding partner, but does not substantially bind other PET binding partners added to the surface of a biosensor. In an alternative embodiment, a nucleic acid microarray (such as an aptamer array) is provided, in which each distinct location within the array contains a different aptamer capture agent. By application of specific capture agents with a microarray spotter onto a biosensor, specific binding substance densities of 10,000 specific binding substances/in² can be obtained. By focusing an illumination beam of a fiber optic probe to interrogate a single microarray location, a biosensor can be used as a label-free microarray readout system.

For the detection of PET binding partners at concentrations of less than about 0.1 ng/ml, one may amplify and transduce binding partners bound to a biosensor into an additional layer on the biosensor surface. The increased mass deposited on the biosensor can be detected as a consequence of increased optical path length. By incorporating greater mass onto a biosensor surface, an optical density of binding partners on the surface is also increased, thus rendering a greater resonant wavelength shift than would occur without the added mass. The addition of mass can be accomplished, for example, enzymatically, through a “sandwich” assay, or by direct application of mass (such as a second capture agent specific for the PET peptide) to the biosensor surface in the form of appropriately conjugated beads or polymers of various size and composition. Since the capture agents are PET-specific, multiple capture agents of different types and specificity can be added together to the captured PETs. This principle has been exploited for other types of optical biosensors to demonstrate sensitivity increases over 1500× beyond sensitivity limits achieved without mass amplification. See, e.g., Jenison et al., “Interference-based detection of nucleic acid targets on optically coated silicon,” Nature Biotechnology, 19: 62-65, 2001.

In an alternative embodiment, a biosensor comprises volume surface-relief volume diffractive structures (a SRVD biosensor). SRVD biosensors have a surface that reflects predominantly at a particular narrow band of optical wavelengths when illuminated with a broad band of optical wavelengths. Where specific capture agents and/or PET binding partners are immobilized on a SRVD biosensor, the reflected wavelength of light is shifted. One-dimensional surfaces, such as thin film interference filters and Bragg reflectors, can select a narrow range of reflected or transmitted wavelengths from a broadband excitation source. However, the deposition of additional material, such as specific capture agents and/or PET binding partners onto their upper surface results only in a change in the resonance linewidth, rather than the resonance wavelength. In contrast, SRVD biosensors have the ability to alter the reflected wavelength with the addition of material, such as specific capture agents and/or binding partners to the surface.

A SRVD biosensor comprises a sheet material having a first and second surface. The first surface of the sheet material defines relief volume diffraction structures. Sheet material can comprise, for example, plastic, glass, semiconductor wafer, or metal film. A relief volume diffractive structure can be, for example, a two-dimensional grating, as described above, or a three-dimensional surface-relief volume diffractive grating. The depth and period of relief volume diffraction structures are less than the resonance wavelength of light reflected from a biosensor. A three-dimensional surface-relief volume diffractive grating can be, for example, a three-dimensional phase-quantized terraced surface relief pattern whose groove pattern resembles a stepped pyramid. When such a grating is illuminated by a beam of broadband radiation, light will be coherently reflected from the equally spaced terraces at a wavelength given by twice the step spacing times the index of refraction of the surrounding medium. Light of a given wavelength is resonantly diffracted or reflected from the steps that are a half-wavelength apart, and with a bandwidth that is inversely proportional to the number of steps. The reflected or diffracted color can be controlled by the deposition of a dielectric layer so that a new wavelength is selected, depending on the index of refraction of the coating.

A stepped-phase structure can be produced first in photoresist by coherently exposing a thin photoresist film to three laser beams, as described previously. See e.g., Cowen, “The recording and large scale replication of crossed holographic grating arrays using multiple beam interferometry,” in International Conference on the Application, Theory, and Fabrication of Periodic Structures, Diffraction Gratings, and Moire Phenomena II, Lerner, ed., Proc. Soc. Photo-Opt. Instrum. Eng., 503, 120-129, 1984; Cowen, “Holographic honeycomb microlens,” Opt. Eng. 24, 796-802 (1985); Cowen & Slafer, “The recording and replication of holographic micropatterns for the ordering of photographic emulsion grains in film systems,” J Imaging Sci. 31, 100-107, 1987. The nonlinear etching characteristics of photoresist are used to develop the exposed film to create a three-dimensional relief pattern. The photoresist structure is then replicated using standard embossing procedures. For example, a thin silver film may be deposited over the photoresist structure to form a conducting layer upon which a thick film of nickel can be electroplated. The nickel “master” plate is then used to emboss directly into a plastic film, such as vinyl, that has been softened by heating or solvent. A theory describing the design and fabrication of three-dimensional phase-quantized terraced surface relief pattern that resemble stepped pyramids is described: Cowen, “Aztec surface-relief volume diffractive structure,” J. Opt. Soc. Am. A, 7:1529 (1990). An example of a three-dimensional phase-quantized terraced surface relief pattern may be a pattern that resembles a stepped pyramid. Each inverted pyramid is approximately 1 micron in diameter. Preferably, each inverted pyramid can be about 0.5 to about 5 microns diameter, including for example, about 1 micron. The pyramid structures can be close-packed so that a typical microarray spot with a diameter of 150-200 microns can incorporate several hundred stepped pyramid structures. The relief volume diffraction structures have a period of about 0.1 to about 1 micron and a depth of about 0.1 to about 1 micron.

One or more specific binding substances, as described above, are immobilized on the reflective material of a SRVD biosensor. One or more specific binding substances can be arranged in microarray of distinct locations, as described above, on the reflective material.

A SRVD biosensor reflects light predominantly at a first single optical wavelength when illuminated with a broad band of optical wavelengths, and reflects light at a second single optical wavelength when one or more specific binding substances are immobilized on the reflective surface. The reflection at the second optical wavelength results from optical interference. A SRVD biosensor also reflects light at a third single optical wavelength when the one or more specific capture agents are bound to their respective PET binding partners, due to optical interference. Readout of the reflected color can be performed serially by focusing a microscope objective onto individual microarray spots and reading the reflected spectrum with the aid of a spectrograph or imaging spectrometer, or in parallel by, for example, projecting the reflected image of the microarray onto an imaging spectrometer incorporating a high resolution color CCD camera.

A SRVD biosensor can be manufactured by, for example, producing a metal master plate, and stamping a relief volume diffractive structure into, for example, a plastic material like vinyl. After stamping, the surface is made reflective by blanket deposition of, for example, a thin metal film such as gold, silver, or aluminum. Compared to MEMS-based biosensors that rely upon photolithography, etching, and wafer bonding procedures, the manufacture of a SRVD biosensor is very inexpensive.

A SWS or SRVD biosensor embodiment can comprise an inner surface. In one preferred embodiment, such an inner surface is a bottom surface of a liquid-containing vessel. A liquid-containing vessel can be, for example, a microtiter plate well, a test tube, a petri dish, or a microfluidic channel. In one embodiment, a SWS or SRVD biosensor is incorporated into a microtiter plate. For example, a SWS biosensor or SRVD biosensor can be incorporated into the bottom surface of a microtiter plate by assembling the walls of the reaction vessels over the resonant reflection surface, so that each reaction “spot” can be exposed to a distinct test sample. Therefore, each individual microtiter plate well can act as a separate reaction vessel. Separate chemical reactions can, therefore, occur within adjacent wells without intermixing reaction fluids and chemically distinct test solutions can be applied to individual wells.

This technology is useful in applications where large numbers of biomolecular interactions are measured in parallel, particularly when molecular labels would alter or inhibit the functionality of the molecules under study. High-throughput screening of pharmaceutical compound libraries with protein targets, and microarray screening of protein-protein interactions for proteomics are examples of applications that require the sensitivity and throughput afforded by the compositions and methods of the invention.

Unlike surface plasmon resonance, resonant mirrors, and waveguide biosensors, the described compositions and methods enable many thousands of individual binding reactions to take place simultaneously upon the biosensor surface. This technology is useful in applications where large numbers of biomolecular interactions are measured in parallel (such as in an array), particularly when molecular labels alter or inhibit the functionality of the molecules under study. These biosensors are especially suited for high-throughput screening of pharmaceutical compound libraries with protein targets, and microarray screening of protein-protein interactions for proteomics. A biosensor of the invention can be manufactured, for example, in large areas using a plastic embossing process, and thus can be inexpensively incorporated into common disposable laboratory assay platforms such as microtiter plates and microarray slides.

Other similar biosensors may also be used in the instant invention. Numerous biosensors have been developed to detect a variety of biomolecular complexes including oligonucleotides, antibody-antigen interactions, hormone-receptor interactions, and enzyme-substrate interactions. In general, these biosensors consist of two components: a highly specific recognition element and a transducer that converts the molecular recognition event into a quantifiable signal. Signal transduction has been accomplished by many methods, including fluorescence, interferometry (Jenison et al., “Interference-based detection of nucleic acid targets on optically coated silicon,” Nature Biotechnology, 19, p. 62-65; Lin et al., “A porous silicon-based optical interferometric biosensor,” Science, 278, p. 840-843, 1997), and gravimetry (A. Cunningham, Bioanalytical Sensors, John Wiley & Sons (1998)). Of the optically-based transduction methods, direct methods that do not require labeling of analytes with fluorescent compounds are of interest due to the relative assay simplicity and ability to study the interaction of small molecules and proteins that are not readily labeled.

These direct optical methods include surface plasmon resonance (SPR) (Jordan & Corn, “Surface Plasmon Resonance Imaging Measurements of Electrostatic Biopolymer Adsorption onto Chemically Modified Gold Surfaces,” Anal. Chem., 69:1449-1456 (1997); plasmom-resonant particles (PRPs) (Schultz et al., Proc. Nat. Acad. Sci., 97: 996-1001 (2000); grating couplers (Morhard et al., “Immobilization of antibodies in micropatterns for cell detection by optical diffraction,” Sensors and Actuators B, 70, p. 232-242, 2000); ellipsometry (Jin et al., “A biosensor concept based on imaging ellipsometry for visualization of biomolecular interactions,” Analytical Biochemistry, 232, p. 69-72, 1995), evanascent wave devices (Huber et al., “Direct optical immunosensing (sensitivity and selectivity),” Sensors and Actuators B, 6, p. 122.126, 1992), resonance light scattering (Bao et al., Anal. Chem., 74:1792-1797 (2002), and reflectometry (Brecht & Gauglitz, “Optical probes and transducers,” Biosensors and Bioelectronics, 10, p. 923-936, 1995). Changes in the optical phenomenon of surface plasmon resonance (SPR) can be used as an indication of real-time reactions between biological molecules. Theoretically predicted detection limits of these detection methods have been determined and experimentally confirmed to be feasible down to diagnostically relevant concentration ranges.

Surface plasmon resonance (SPR) has been successfully incorporated into an immunosensor format for the simple, rapid, and nonlabeled assay of various biochemical analytes. Proteins, complex conjugates, toxins, allergens, drugs, and pesticides can be determined directly using either natural antibodies or synthetic receptors with high sensitivity and selectivity as the sensing element. Immunosensors are capable of real-time monitoring of the antigen-antibody reaction. A wide range of molecules can be detected with lower limits ranging between 10⁻⁹ and 10⁻¹³ mol/L. Several successful commercial developments of SPR immunosensors are available and their web pages are rich in technical information. Wayne et al. (Methods 22: 77-91, 2000) reviewed and highlighted many recent developments in SPR-based immunoassay, functionalizations of the gold surface, novel receptors in molecular recognition, and advanced techniques for sensitivity enhancement.

Utilization of the optical phenomenon surface plasmon resonance (SPR) has seen extensive growth since its initial observation by Wood in 1902 (Phil. Mag. 4 (1902), pp. 396-402). SPR is a simple and direct sensing technique that can be used to probe refractive index (η) changes that occur in the very close vicinity of a thin metal film surface (Otto Z. Phys. 216 (1968), p. 398). The sensing mechanism exploits the properties of an evanescent field generated at the site of total internal reflection. This field penetrates into the metal film, with exponentially decreasing amplitude from the glass-metal interface. Surface plasmons, which oscillate and propagate along the upper surface of the metal film, absorb some of the plane-polarized light energy from this evanescent field to change the total internal reflection light intensity Iᵣ. A plot of Iᵣ versus incidence (or reflection) angle θ produces an angular intensity profile that exhibits a sharp dip. The exact location of the dip minimum (or the SPR angle θᵣ) can be determined by using a polynomial algorithm to fit the Iᵣ signals from a few diodes close to the minimum. The binding of molecules on the upper metal surface causes a change in η of the surface medium that can be observed as a shift in θᵣ.

The potential of SPR for biosensor purposes was realized in 1982-1983 by Liedberg et al., who adsorbed an immunoglobulin G (IgG) antibody overlayer on the gold sensing film, resulting in the subsequent selective binding and detection of IgG (Nylander et al., Sens. Actuators 3 (1982), pp. 79-84; Liedberg et al., Sens. Actuators 4 (1983), pp. 229-304). The principles of SPR as a biosensing technique have been reviewed previously (Daniels et al., Sens. Actuators 15 (1988), pp. 11-18; VanderNoot and Lai, Spectroscopy 6 (1991), pp. 28-33; Lundström Biosens. Bioelectron. 9 (1994), pp. 725-736; Liedberg et al., Biosens. Bioelectron. 10 (1995); Morgan et al., Clin. Chem. 42 (1996), pp. 193-209; Tapuchi et al., S. Afr. J. Chem. 49 (1996), pp. 8-25). Applications of SPR to biosensing were demonstrated for a wide range of molecules, from virus particles to sex hormone-binding globulin and syphilis. Most importantly, SPR has an inherent advantage over other types of biosensors in its versatility and capability of monitoring binding interactions without the need for fluorescence or radioisotope labeling of the biomolecules. This approach has also shown promise in the real-time determination of concentration, kinetic constant, and binding specificity of individual biomolecular interaction steps. Antibody-antigen interactions, peptide/protein-protein interactions, DNA hybridization conditions, biocompatibility studies of polymers, biomolecule-cell receptor interactions, and DNA/receptor-ligand interactions can all be analyzed (Pathak and Savelkoul, Immunol. Today 18 (1997), pp. 464-467). Commercially, the use of SPR-based immunoassay has been promoted by companies such as Biacore (Uppsala, Sweden) (Jönsson et al., Ann. Biol. Clin. 51 (1993), pp. 19-26), Windsor Scientific (U.K.) (WWW URL for Windsor Scientific IBIS Biosensor), Quantech (Minnesota) (WWW URL for Quantech), and Texas Instruments (Dallas, Tex.) (WWW URL for Texas Instruments).

In yet another embodiment, a fluorescent polymer superquenching-based bioassays as disclosed in WO 02/074997 may be used for detecting binding of the unlabeled PET to its capture agents. In this embodiment, a capture agent that is specific for both a target PET peptide and a chemical moiety is used. The chemical moiety includes (a) a recognition element for the capture agent, (b) a fluorescent property-altering element, and (c) a tethering element linking the recognition element and the property-altering element. A composition comprising a fluorescent polymer and the capture agent are co-located on a support. When the chemical moiety is bound to the capture agent, the property-altering element of the chemical moiety is sufficiently close to the fluorescent polymer to alter (quench) the fluorescence emitted by the polymer. When an analyte sample is introduced, the target PET peptide, if present, binds to the capture agent, thereby displacing the chemical moiety from the receptor, resulting in de-quenching and an increase of detected fluorescence. Assays for detecting the presence of a target biological agent are also disclosed in the application.

In another related embodiment, the binding event between the capture agents and the PET can be detected by using a water-soluble luminescent quantum dot as described in U.S. 2003/0008414A1. In one embodiment, a water-soluble luminescent semiconductor quantum dot comprises a core, a cap and a hydrophilic attachment group. The “core” is a nanoparticle-sized semiconductor. While any core of the IIB-VIB, IIIB-VB or IVB-IVB semiconductors can be used in this context, the core must be such that, upon combination with a cap, a luminescent quantum dot results. A IIB-VIB semiconductor is a compound that contains at least one element from Group IEB and at least one element from Group VIB of the periodic table, and so on. Preferably, the core is a IIB-VIB, IIIB-VB or IVB-IVB semiconductor that ranges in size from about 1 nm to about 10 nm. The core is more preferably a IIB-VIB semiconductor and ranges in size from about 2 nm to about 5 nm. Most preferably, the core is CdS or CdSe. In this regard, CdSe is especially preferred as the core, in particular at a size of about 4.2 nm.

The “cap” is a semiconductor that differs from the semiconductor of the core and binds to the core, thereby forming a surface layer on the core. The cap must be such that, upon combination with a given semiconductor core, results in a luminescent quantum dot. The cap should passivate the core by having a higher band gap than the core. In this regard, the cap is preferably a IIB-VIB semiconductor of high band gap. More preferably, the cap is ZnS or CdS. Most preferably, the cap is ZnS. In particular, the cap is preferably ZnS when the core is CdSe or CdS and the cap is preferably CdS when the core is CdSe.

The “attachment group” as that term is used herein refers to any organic group that can be attached, such as by any stable physical or chemical association, to the surface of the cap of the luminescent semiconductor quantum dot and can render the quantum dot water-soluble without rendering the quantum dot no longer luminescent. Accordingly, the attachment group comprises a hydrophilic moiety. Preferably, the attachment group enables the hydrophilic quantum dot to remain in solution for at least about one hour, one day, one week, or one month. Desirably, the attachment group is attached to the cap by covalent bonding and is attached to the cap in such a manner that the hydrophilic moiety is exposed. Preferably, the hydrophilic attachment group is attached to the quantum dot via a sulfur atom. More preferably, the hydrophilic attachment group is an organic group comprising a sulfur atom and at least one hydrophilic attachment group. Suitable hydrophilic attachment groups include, for example, a carboxylic acid or salt thereof, a sulfonic acid or salt thereof, a sulfamic acid or salt thereof, an amino substituent, a quaternary ammonium salt, and a hydroxy. The organic group of the hydrophilic attachment group of the present invention is preferably a C1-C6 alkyl group or an aryl group, more preferably a C1-C6 alkyl group, even more preferably a C1-C3 alkyl group. Therefore, in a preferred embodiment, the attachment group of the present invention is a thiol carboxylic acid or thiol alcohol. More preferably, the attachment group is a thiol carboxylic acid. Most preferably, the attachment group is mercaptoacetic acid.

Accordingly, a preferred embodiment of a water-soluble luminescent semiconductor quantum dot is one that comprises a CdSe core of about 4.2 nm in size, a ZnS cap and an attachment group. Another preferred embodiment of a watersoluble luminescent semiconductor quantum dot is one that comprises a CdSe core, a ZnS cap and the attachment group mercaptoacetic acid. An especially preferred water-soluble luminescent semiconductor quantum dot comprises a CdSe core of about 4.2 nm, a ZnS cap of about 1 nm and a mercaptoacetic acid attachment group.

The capture agent of the instant invention can be attached to the quantum dot via the hydrophilic attachment group and forms a conjugate. The capture agent can be attached, such as by any stable physical or chemical association, to the hydrophilic attachment group of the water-soluble luminescent quantum dot directly or indirectly by any suitable means, through one or more covalent bonds, via an optional linker that does not impair the function of the capture agent or the quantum dot. For example, if the attachment group is mercaptoacetic acid and a nucleic acid biomolecule is being attached to the attachment group, the linker preferably is a primary amine, a thiol, streptavidin, neutravidin, biotin, or a like molecule. If the attachment group is mercaptoacetic acid and a protein biomolecule or a fragment thereof is being attached to the attachment group, the linker preferably is strepavidin, neutravidin, biotin, or a like molecule.

By using the quantum dot-capture agent conjugate, a PET-containing sample, when contacted with a conjugate as described above, will promote the emission of luminescence when the capture agent of the conjugate specifically binds to the PET peptide. This is particularly useful when the capture agent is a nucleic acid aptamer or an antibody. When the aptamer is used, an alternative embodiment may be employed, in which a fluorescent quencher may be positioned adjacent to the quantum dot via a self-pairing stem-loop structure when the aptamer is not bound to a PET-containing sequence. When the aptamer binds to the PET, the stem-loop structure is opened, thus releasing the quenching effect and generates luminescence.

In another related embodiment, arrays of nanosensors comprising nanowires or nanotubes as described in U.S. 2002/0117659A1 may be used for detection and/or quantitation of PET-capture agent interaction. Briefly, a “nanowire” is an elongated nanoscale semiconductor, which can have a cross-sectional dimension of as thin as 1 nanometer. Similarly, a “nanotube” is a nanowire that has a hollowed-out core, and includes those nanotubes know to those of ordinary skill in the art. A “wire” refers to any material having a conductivity at least that of a semiconductor or metal. These nanowires/nanotubes may be used in a system constructed and arranged to determine an analyte (e.g., PET peptide) in a sample to which the nanowire(s) is exposed. The surface of the nanowire is functionalized by coating with a capture agent. Binding of an analyte to the functionalized nanowire causes a detectable change in electrical conductivity of the nanowire or optical properties. Thus, presence of the analyte can be determined by determining a change in a characteristic in the nanowire, typically an electrical characteristic or an optical characteristic. A variety of biomolecular entities can be used for coating, including, but not limited to, amino acids, proteins, sugars, DNA, antibodies, antigens, and enzymes, etc. For more details such as construction of nanowires, functionalization with various biomolecules (such as the capture agents of the instant invention), and detection in nanowire devices, see U.S. 2002/0117659A1 (incorporated by reference). Since multiple nanowires can be used in parelle, each with a different capture agent as the functionalized group, this technology is ideally suited for large scale arrayed detection of PET-containing peptides in biological samples without the need to label the PET peptides. This nanowire detection technology has been successfully used to detect pH change (H⁺ binding), biotin-streptavidin binding, antibody-antigen binding, metal (Ca²⁺) binding with picomolar sensitivity and in real time (Cui et al., Science 293: 1289-1292).

Matrix-assisted laser desorption/ionization time-of-flight mass spectrometry (MALDI-TOF MS), uses a laser pulse to desorb proteins from the surface followed by mass spectrometry to identify the molecular weights of the proteins (Gilligan et al., Mass spectrometry after capture and small-volume elution of analyte from a surface plasmon resonance biosensor. Anal. Chem. 74 (2002), pp. 2041-2047). Because this method only measures the mass of proteins at the interface, and because the desorption protocol is sufficiently mild that it does not result in fragmentation, MALDI can provide straightforward useful information such as confirming the identity of the bound PET peptide, or any enzymatic modification of a PET peptide. For this matter, MALDI can be used to identify proteins that are bound to immobilized capture agents. An important technique for identifying bound proteins relies on treating the array (and the proteins that are selectively bound to the array) with proteases and then analyzing the resulting peptides to obtain sequence data.

IV. Samples and Their Preparation

The capture agents or an array of capture agents typically are contacted with a sample, e.g., a biological fluid, a water sample, or a food sample, which has been fragmented to generate a collection of peptides, under conditions suitable for binding a PET corresponding to a protein of interest.

Samples to be assayed using the capture agents of the present invention may be drawn from various physiological, environmental or artificial sources. In particular, physiological samples such as body fluids or tissue samples of a patient or an organism may be used as assay samples. Such fluids include, but are not limited to, saliva, mucous, sweat, whole blood, serum, urine, amniotic fluid, genital fluids, fecal material, marrow, plasma, spinal fluid, pericardial fluids, gastric fluids, abdominal fluids, peritoneal fluids, pleural fluids and extraction from other body parts, and secretion from other glands. Alternatively, biological samples drawn from cells taken from the patient or grown in culture may be employed. Such samples include supernatants, whole cell lysates, or cell fractions obtained by lysis and fractionation of cellular material. Extracts of cells and fractions thereof, including those directly from a biological entity and those grown in an artificial environment, can also be used. In addition, a biological sample can be obtained and/or deribed from, for example, blood, plasma, serum, gastrointestinal secretions, homogenates of tissues or tumors, synovial fluid, feces, saliva, sputum, cyst fluid, amniotic fluid, cerebrospinal fluid, peritoneal fluid, lung lavage fluid, semen, lymphatic fluid, tears, or prostatitc fluid.

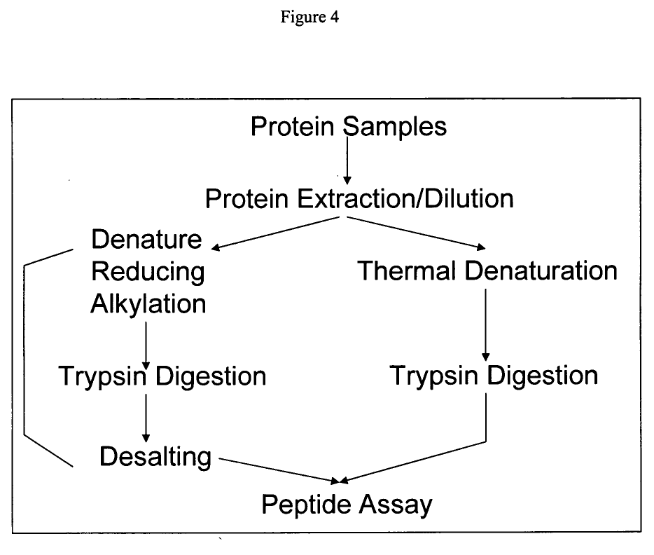

A general scheme of sample preparation prior to its use in the methods of the instant invention is described in FIG. 4. Briefly, a sample can be pretreated by extraction and/or dilution to minimize the interference from certain substances present in the sample. The sample can then be either chemically reduced, denatured, alkylated, or subjected to thermo-denaturation. Regardless of the denaturation step, the denatured sample is then digested by a protease, such as trypsin, before it is used in subsequent assays. A desalting step may also be added just after protease digestion if chemical denaturation if used. This process is generally simple, robust and reproducible, and is generally applicable to main sample types including serum, cell lysates and tissues.

The sample may be pre-treated to remove extraneous materials, stabilized, buffered, preserved, filtered, or otherwise conditioned as desired or necessary. Proteins in the sample typically are fragmented, either as part of the methods of the invention or in advance of performing these methods. Fragmentation can be performed using any art-recognized desired method, such as by using chemical cleavage (e.g., cyanogen bromide); enzymatic means (e.g., using a protease such as trypsin, chymotrypsin, pepsin, papain, carboxypeptidase, calpain, subtilisin, gluc-C, endo lys-C and proteinase K, or a collection or sub-collection thereof); or physical means (e.g., fragmentation by physical shearing or fragmentation by sonication). As used herein, the terms “fragmentation” “cleavage,” “proteolytic cleavage,” “proteolysis” “restriction” and the like are used interchangeably and refer to scission of a chemical bond, typically a peptide bond, within proteins to produce a collection of peptides (i.e., protein fragments).

The purpose of the fragmentation is to generate peptides comprising PET which are soluble and available for binding with a capture agent. In essence, the sample preparation is designed to assure to the extent possible that all PET present on or within relevant proteins that may be present in the sample are available for reaction with the capture agents. This strategy can avoid many of the problems encountered with previous attempts to design protein chips caused by protein-protein complexation, post translational modifications and the like.

In one embodiment, the sample of interest is treated using a pre-determined protocol which: (A) inhibits masking of the target protein caused by target protein-protein non covalent or covalent complexation or aggregation, target protein degradation or denaturing, target protein post-translational modification, or environmentally induced alteration in target protein tertiary structure, and (B) fragments the target protein to, thereby, produce at least one peptide epitope (i.e., a PET) whose concentration is directly proportional to the true concentration of the target protein in the sample. The sample treatment protocol is designed and empirically tested to result reproducibly in the generation of a PET that is available for reaction with a given capture agent. The treatment can involve protein separations; protein fractionations; solvent modifications such as polarity changes, osmolarity changes, dilutions, or pH changes; heating; freezing; precipitating; extractions; reactions with a reagent such as an endo-, exo- or site specific protease; non proteolytic digestion; oxidations; reductions; neutralization of some biological activity, and other steps known to one of skill in the art.

For example, the sample may be treated with an alkylating agent and a reducing agent in order to prevent the formation of dimers or other aggregates through disulfide/dithiol exchange. The sample of PET-containing peptides may also be treated to remove secondary modifications, including but are not limited to, phosphorylation, methylation, glycosylation, acetylation, prenylation, using, for example, respective modification-specific enzymes such as phosphatases, etc.

In one embodiment, proteins of a sample will be denatured, reduced and/or alkylated, but will not be proteolytically cleaved. Proteins can be denatured by thermal denaturation or organic solvents, then subjected to direct detection or optionally, further proteolytic cleavage.

The use of thermal denaturation (50-90° C. for about 20 minutes) of proteins prior to enzyme digestion in solution is preferred over chemical denaturation (such as 6-8 M guanidine HCl or urea) because it does not require purification/concentration, which might be preferred or required prior to subsequent analysis. Park and Russell reported that enzymatic digestions of proteins that are resistant to proteolysis are significantly enhanced by thermal denaturation (Anal. Chem., 72 (11): 2667-2670, 2000). Native proteins that are sensitive to proteolysis show similar or just slightly lower digestion yields following thermal denaturation. Proteins that are resistant to digestion become more susceptible to digestion, independent of protein size, following thermal denaturation. For example, amino acid sequence coverage from digest fragments increases from 15 to 86% in myoglobin and from 0 to 43% in ovalbumin. This leads to more rapid and reliable protein identification by the instant invention, especially to protease resistant proteins.

Although some proteins aggregate upon thermal denaturation, the protein aggregates are easily digested by trypsin and generate sufficient numbers of digest fragments for protein identification. In fact, protein aggregation may be the reason thermal denaturation facilitates digestion in most cases. Protein aggregates are believed to be the oligomerization products of the denatured form of protein (Copeland, R. A. Methods for Protein Analysis; Chapman & Hall: New York, N.Y., 1994). In general, hydrophobic parts of the protein are located inside and relatively less hydrophobic parts of the protein are exposed to the aqueous environment. During the thermal denaturation, intact proteins are gradually unfolded into a denatured conformation and sufficient energy is provided to prevent a fold back to its native conformation. The probability for interactions with other denatured proteins is increased, thus allowing hydrophobic interactions between exposed hydrophobic parts of the proteins. In addition, protein aggregates of the denatured protein can have a more protease-labile structure than nondenatured proteins because more cleavage sites are exposed to the environment. Protein aggregates are easily digested, so that protein aggregates are not observed at the end of 3 h of trypsin digestion (Park and Russell, Anal. Chem., 72 (11): 2667-2670, 2000). Moreover, trypsin digestion of protein aggregates generates more specific cleavage products.

Ordinary proteases such as trypsin may be used after denaturation. The process may be repeated by one or more rounds after the first round of denaturation and digestion. Alternatively, this thermal denaturation process can be further assisted by using thermophilic trypsin-like enzymes, so that denaturation and digestion can be done simultaneously. For example, Nongporn Towatana et al. (J of Bioscience and Bioengineering 87(5): 581-587, 1999) reported the purification to apparent homogeneity of an alkaline protease from culture supernatants of Bacillus sp. PS719, a novel alkaliphilic, thermophilic bacterium isolated from a thermal spring soil sample. The protease exhibited maximum activity towards azocasein at pH 9.0 and at 75° C. The enzyme was stable in the pH range 8.0 to 10.0 and up to 80° C. in the absence of Ca²⁺. This enzyme appears to be a trypsin-like serine protease, since phenylmethylsulfonyl fluoride (PMSF) and 3,4-dichloroisocoumarin (DCI) in addition to N-α-p-tosyl-L-lysine chloromethyl ketone (TLCK) completely inhibited the activity. Among the various oligopeptidyl-p-nitroanilides tested, the protease showed a preference for cleavage at arginine residues on the carboxylic side of the scissile bond of the substrate, liberating p-nitroaniline from N-carbobenzoxy (CBZ)-L-arginine-p-nitroanilide with the Kₘ and Vₘₐₓ values of 0.6 mM and 1.0 pmol min⁻¹ mg protein⁻¹, respectively.

Alternatively, existing proteases may be chemically modified to achieve enhanced thermostability for use in this type of application. Mozhaev et al. (Eur J. Biochem. 173(1):147-54, 1988) experimentally verified the idea presented earlier that the contact of nonpolar clusters located on the surface of protein molecules with water destabilizes proteins. It was demonstrated that protein stabilization could be achieved by artificial hydrophilization of the surface area of protein globules by chemical modification. Two experimental systems were studied for the verification of the hydrophilization approach. In one experiment, the surface tyrosine residues of trypsin were transformed to aminotyrosines using a two-step modification procedure: nitration by tetranitromethane followed by reduction with sodium dithionite. The modified enzyme was much more stable against irreversible thermo-inactivation: the stabilizing effect increased with the number of aminotyrosine residues in trypsin and the modified enzyme could become even 100 times more stable than the native one. In another experiment, alpha-chymotrypsin was covalently modified by treatment with anhydrides or chloroanhydrides of aromatic carboxylic acids. As a result, different numbers of additional carboxylic groups (up to five depending on the structure of the modifying reagent) were introduced into each Lys residue modified. Acylation of all available amino groups of alpha-chymotrypsin by cyclic anhydrides of pyromellitic and mellitic acids resulted in a substantial hydrophilization of the protein as estimated by partitioning in an aqueous Ficoll-400/Dextran-70 biphasic system. These modified enzyme preparations were extremely stable against irreversible thermal inactivation at elevated temperatures (65-98° C.); their thermostability was practically equal to the stability of proteolytic enzymes from extremely thermophilic bacteria, the most stable proteinases known to date. Similar approaches may be used to any other chosen proteases for the subject method.

In other embodiments, samples can be pre-treated with reducing agents such as b-mercaptoethanol or DTT to reduce the disulfide bonds to facilitate digestion.

Fractionation may be performed using any single or multidimentional chromatography, such as reversed phase chromatography (RPC), ion exchange chromatography, hydrophobic interaction chromatography, size exclusion chromatography, or affinity fractionation such as immunoaffinity and immobilized metal affinity chromatography. Preferably, the fractionation involves surface-mediated selection strategies. Electrophoresis, either slab gel or capillary electrophoresis, can also be used to fractionate the peptides in the sample. Examples of slab gel electrophoretic methods include sodium dodecyl sulfate polyacrylamide gel electrophoresis (SDS-PAGE) and native gel electrophoresis. Capillary electrophoresis methods that can be used for fractionation include capillary gel electrophoresis (CGE), capillary zone electrophoresis (CZE) and capillary electrochromatography (CEC), capillary isoelectric focusing, immobilized metal affinity chromatography and affinity electrophoresis.

Protein precipitation may be performed using techniques well known in the art. For example, precipitation may be achieved using known precipitants, such as potassium thiocyanate, trichloroacetic acid and ammonium sulphate.

Subsequent to fragmentation, the sample may be contacted with the capture agents of the present invention, e.g., capture agents immobilized on a planar support or on a bead, as described herein. Alternatively, the fragmented sample (containing a collection of peptides) may be fractionated based on, for example, size, post-translational modifications (e.g., glycosylation or phosphorylation) or antigenic properties, and then contacted with the capture agents of the present invention, e.g., capture agents immobilized on a planar support or on a bead.

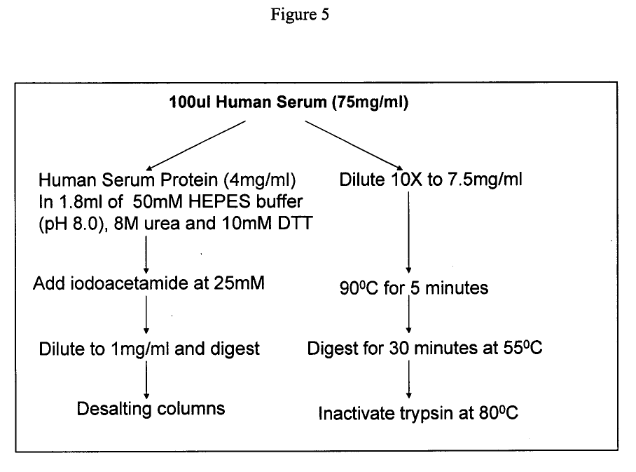

FIG. 5 provides an illustrative example of serum sample pre-treatment using either the thermo-denaturation or the chemical denaturation. Briefly, for thermo-denaturation, 100 μL of human serum (about 75 mg/mL total protein) is first diluted 10-fold to about 7.5 mg/mL. The diluted sample is then heated to 90° C. for 5 minutes to denature the proteins, followed by 30 minutes of trypsin digestion at 55° C. The trypsin is inactivated at 80° C. after the digestion.

For chemical denaturation, about 1.8 mL of human serum proteins diluted to about 4 mg/mL is denatured in a final concentration of 50 mM HEPES buffer (pH 8.0), 8M urea and 10 mM DTT. Iodoacetamide is then added to 25 mM final concentration. The denatured sample is then further diluted to about 1 mg/mL for protease digestion. The digested sample will pass through a desalting column before being used in subsequent assays.

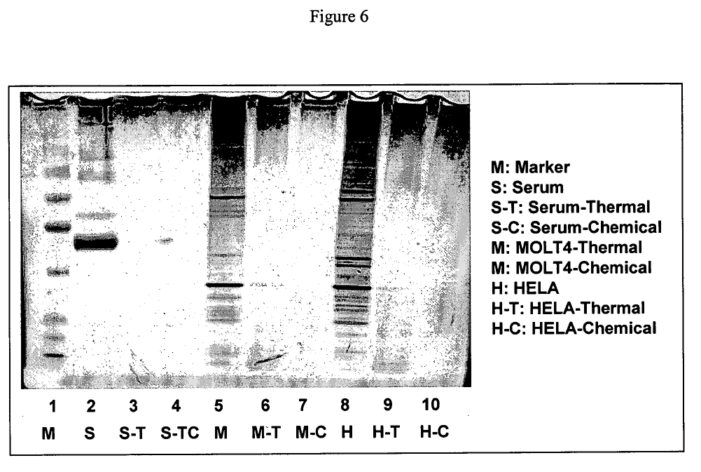

FIG. 6 shows the result of thermo-denaturation and chemical denaturation of serum proteins, cell lysates (MOLT4 and Hela cells). It is evident that denaturation was successful for the majority, if not all of the proteins in both the thermo- and chemical-denaturation lanes, and both methods achieved comparable results in terms of protein denaturation and fragmentation.

Example 9 below also describes a preferred sample treatment procedure. All examples are for illustrative purpose only and are by no means limiting. Minor alterations of the protocol depending on specific uses can be easily achieved for optimal results in individual assays.

V. Selection of PET

One advantages of the PET of the instant invention is that PET can be determined in sillico and generated in vitro (such as by peptide synthesis) without cloning or purifying the protein it belongs. PET is also advantageous over the full-length tryptic fragments (or for that matter, any other fragments that predictably results from any other treatments) since full-length tryptic fragments tend to contain one or more PETs themselves, though the tryptic fragment itself may be unique simply because of its length (the longer a stretch of peptide, the more likely it will be unique). A direct implication is that, by using relatively short and unique PETs rather than the full-length (tryptic) peptide fragments, the method of the instant invention has greatly reduced, if not completely eliminated, the risk of having multiple antibodies with unique specificities against the same peptide fragment—a source of antibody cross-reactivity. An additional advantage may be added due to the PET selection process, such as the nearest-neighbor analysis and ranking prioritization (see below), which further eliminates the chance of cross-reactivity. All these features make the PET-based methods particularly suitable for genome-wise analysis using multiplexing techniques.

The PET of the instant invention can be selected in various ways. In the simplest embodiment, the PET for a given organism or biological sample can be generated or identified by a brute force search of the relevant database, using all theoretically possible PET with a given length. This process is preferably carried out computationaly using, for example, any of the sequence search tools available in the art or variations thereof. For example, to identify PET of 5 amino acids in length (a total of 3.2 million possible PET candidates, see table 2.2.2 of the parent applications, incorporated herein by reference), each of the 3.2 million candidates may be used as a query sequence to search against the human proteom as described below. Any candidate that has more than one hit (found in two or more proteins) is immediately eliminated before further searching is done. At the end of the search, a list of human proteins that have one or more PETs can be obtained (see Example 1 of the parent application, incorporated herein by reference). The same or similar procedure can be used for any pre-determined organism or database.

For example, PETs for each human protein can be identified using the following procedure. A Perl program is developed to calculate the occurrence of all possible peptides, given by 20^{N}, of defined length N (amino acids) in human proteins. For example, the total tag space is 160,000 (20⁴) for tetramer peptides, 3.2 M (20⁵) for pentamer peptides, and 64 M (20⁶) for hexamer peptides, so on. Predicted human protein sequences are analyzed for the presence or absence of all possible peptides of N amino acids. PET are the peptide sequences that occur only once in the human proteome. Thus the presence of a specific PET is an intrinsic property of the protein sequence and is operational independent. According to this approach, a definitive set of PETs can be defined and used regardless of the sample processing procedure (operational independence).

In one embodiment, to speed up the searching process, computer algorithms may be developed or modified to eliminate unnecessary searches before the actual search begins.

Using the example above, two highly related (say differ only in a few amino acid positions) human proteins may be aligned, and a large number of candidate PET can be eliminated based on the sequence of the identical regions. For example, if there is a stretch of identical sequence of 20 amino acids, then sixteen 5-amino acid PETs can be eliminated without searching, by virtue of their simultaneous appearance in two non-identical human proteins. This elimination process can be continued using as many highly related protein pairs or families as possible, such as the evolutionary conserved proteins such as histones, globins, etc.

In another embodiment, the identified PET for a given protein may be rank-ordered based on certain criteria, so that higher ranking PETs are preferred to be used in generating specific capture agents.

For example, certain PET may naturally exist on protein surface, thus making good candidates for being a soluble peptide when digested by a protease. On the other hand, certain PET may exist in an internal or core region of a protein, and may not be readily soluble even after digestion. Such solubility property may be evaluated by available softwares. The solvent accessibility method described in Boger, J., Emini, E. A. & Schmidt, A., Surface probability profile-An heuristic approach to the selection of synthetic peptide antigens, Reports on the Sixth International Congress in Immunology (Toronto) 1986 p. 250 also may be used to identify PETs that are located on the surface of the protein of interest. The package MOLMOL (Koradi, R. et al. (1996) J. Mol. Graph. 14:51-55) and Eisenhaber's ASC method (Eisenhaber and Argos (1993) J. Comput. Chem. 14:1272-1280; Eisenhaber et al. (1995) J. Comput. Chem. 16:273-284) may also be used. Surface PETs generally have higher ranking than internal PETs. In one embodiment, the logP or logD values that can be calculated for a PET, or proteolytic fragment containing a PET, can be calculated and used to rank order the PET's based on likely solubility under conditions that a protein sample is to be contacted with a capture agent.

Regardless of the manner the PETs are generated, an ideal PET preferably is 8 amino acids in length, and the parental tryptic peptide should be smaller than 20 amino acid long. This is because antibodies typically recognize peptide epitopes of 4-8 amino acids, thus peptides of 12-20 amino acids are conventionally used for antibody production.

Since trypsin is a preferred digestion enzyme in certain embodiments, a PET in these embodiments should not contain K or R in the middle of the sequence so that the PET will not be cleaved by trypsin during sample preparation. In a more general sense, the selected PET should not contain or overlap a digestion site such that the PET is expected to be destroyed after digestion, unless an assay specifically prefer that a PET be destroyed after digestion.

In addition, an ideal PET preferably does not have hydrophobic parental tryptic peptide, is highly antigenic, and has the smallest numbers (preferably none) of closest related peptides (nearest neighbor peptides or NNP) defined by nearest neighbor analysis.

Any PET may also be associated with an annotation, which may contain useful information such as: whether the PET may be destroyed by a certain protease (such as trypsin), whether it is likely to appear on a digested peptide with a relatively rigid or flexible structure, etc. These characteristics may help to rank order the PETs for use if generating specific capture agents, especially when there are a large number of PETs associated with a given protein. Since PET may change depending on particular use in a given organism, ranking order may change depending on specific usages. A PET may be low ranking due to its probability of being destroyed by a certain protease may rank higher in a different fragmentation scheme using a different protease.

In another embodiment, the computational algorithm for selecting optimal PET from a protein for antibody generation takes antibody-peptide interaction data into consideration. A process such as Nearest-Neighbor Analysis (NNA), can be used to select most unique PET for each protein. Each PET in a protein is given a relative score, or PET Uniqueness Index, that is based on the number of nearest neighbors it has. The higher the PET Uniqueness Index, the more unique the PET is. The PET Uniqueness Index can be calculated using an Amino Acid Replacement Matrix such as the one in Table VIII of Getzoff, E D, Tainer J A and Lerner R A. The chemistry and meachnism of antibody binding to protein antigens. 1988. Advances. Immunol. 43: 1-97. In this matrix, the replaceability of each amino acid by the remaining 19 amino acids was calculated based on experimental data on antibody cross-reactivity to a large number of peptides of single mutations (replacing each amino acid in a peptide sequence by the remaining 19 amino acids). For example, each octamer PET from a protein is compared to 8.7 million octamers present in human proteome and a PET Uniqueness Index is calculated. This process not only selects the most unique PET for particular protein, it also identifies Nearest Neighbor Peptides for this PET. This becomes important for defining cross-reactivity of PET-specific antibodies since Nearest Neighbor Peptides are the ones most likely will cross-react with particular antibody.

Besides PET Uniqueness Index, the following parameters for each PET may also be calculated and help to rank the PETs:
a) PET Solubility Index: which involves calculating LogP and LogD of the PET.
b) PET Hydrophobicity & water accessibility: only hydrophilic peptides and peptides with good water accessibility will be selected.
c) PET Length: since longer peptides tend to have conformations in solution, we use PET peptides with defined length of 8 amino acids. PET-specific antibodies will have better defined specificity due to limited number of epitopes in a shorter peptide sequences. This is very important for multiplexing assays using these antibodies. In one embodiment, only antibodies generated by this way will be used for multiplexing assays.
d) Evolutionary Conservation Index: each human PET will be compared with other species to see whether a PET sequence is conserved cross species. Ideally, PET with minimal conservation, for example, between mouse and human sequences will be selected. This will maximize the possibility to generate good immunoresponse and monoclonal antibodies in mouse.
   VI. Applications of the Invention

A. Investigative and Diagnostic Applications

The capture agents of the present invention provide a powerful tool in probing living systems and in diagnostic applications (e.g., clinical, environmental and industrial, and food safety diagnostic applications). For clinical diagnostic applications, the capture agents are designed such that they bind to one or more PET corresponding to one or more diagnostic targets (e.g., a disease related protein, collection of proteins, or pattern of proteins). Specific individual disease related proteins include, for example, prostate-specific antigen (PSA), prostatic acid phosphatase (PAP) or prostate specific membrane antigen (PSMA) (for diagnosing prostate cancer); Cyclin E for diagnosing breast cancer; Annexin, e.g., Annexin V (for diagnosing cell death in, for example, cancer, ischemia, or transplant rejection); or β-amyloid plaques (for diagnosing Alzheimer's Disease).

Thus, PETs and the capture agents of the present invention may be used as a source of surrogate markers. For example, they can be used as markers of disorders or disease states, as markers for precursors of disease states, as markers for predisposition of disease states, as markers of drug activity, or as markers of the pharmacogenomic profile of protein expression.

As used herein, a “surrogate marker” is an objective biochemical marker which correlates with the absence or presence of a disease or disorder, or with the progression of a disease or disorder (e.g., with the presence or absence of a tumor). The presence or quantity of such markers is independent of the causation of the disease. Therefore, these markers may serve to indicate whether a particular course of treatment is effective in lessening a disease state or disorder. Surrogate markers are of particular use when the presence or extent of a disease state or disorder is difficult to assess through standard methodologies (e.g., early stage tumors), or when an assessment of disease progression is desired before a potentially dangerous clinical endpoint is reached (e.g., an assessment of cardiovascular disease may be made using a PET corresponding to a protein associated with a cardiovascular disease as a surrogate marker, and an analysis of HIV infection may be made using a PET corresponding to an HIV protein as a surrogate marker, well in advance of the undesirable clinical outcomes of myocardial infarction or fully-developed AIDS). Examples of the use of surrogate markers in the art include: Koomen et al. (2000) J. Mass. Spectrom. 35:258-264; and James (1994) AIDS Treatment News Archive 209.

Perhaps the most significant use of the invention is that it enables practice of a powerful new protein expression analysis technique: analyses of samples for the presence of specific combinations of proteins and specific levels of expression of combinations of proteins. This is valuable in molecular biology investigations generally, and particularly in development of novel assays. Thus, this invention permits one to identify proteins, groups of proteins, and protein expression patterns present in a sample which are characteristic of some disease, physiologic state, or species identity. Such multiparametric assay protocols may be particularly informative if the proteins being detected are from disconnected or remotely connected pathways. For example, the invention might be used to compare protein expression patterns in tissue, urine, or blood from normal patients and cancer patients, and to discover that in the presence of a particular type of cancer a first group of proteins are expressed at a higher level than normal and another group are expressed at a lower level. As another example, the protein chips might be used to survey protein expression levels in various strains of bacteria, to discover patterns of expression which characterize different strains, and, to determine which strains are susceptible to which antibiotic. Furthermore, the invention enables production of specialty assay devices comprising arrays or other arrangements of capture agents for detecting specific patterns of specific proteins. Thus, to continue the example, in accordance with the practice of the invention, one can produce a chip which can be exposed to a cell lysate preparation from a patient or a body fluid to reveal the presence or absence or pattern of expression informative that the patient is cancer free, or is suffering from a particular cancer type. Alternatively, one might produce a protein chip that would be exposed to a sample and read to indicate the species of bacteria in an infection and the antibiotic that will destroy it.

A junction PET is a peptide which spans the region of a protein corresponding to a splice site of the RNA which encodes it. Capture agents designed to bind to a junction PET may be included in such analyses to detect splice variants as well as gene fusions generated by chromosomal rearrangements, e.g., cancer-associated chromosomal rearrangements. Detection of such rearrangements may lead to a diagnosis of a disease, e.g., cancer. It is now becoming apparent that splice variants are common and that mechanisms for controlling RNA splicing have evolved as a control mechanism for various physiological processes. The invention permits detection of expression of proteins encoded by such species, and correlation of the presence of such proteins with disease or abnormality. Examples of cancer-associated chromosomal rearrangements include: translocation t(16; 21)(p11; q22) between genes FUS-ERG associated with myeloid leukemia and non-lymphocytic, acute leukemia (see Ichikawa H. et al. (1994) Cancer Res. 54(11):2865-8); translocation t(21; 22)(q22; q12) between genes ERG-EWS associated with Ewing's sarcoma and neuroepithelioma (see Kaneko Y. et al. (1997) Genes Chromosomes Cancer 18(3):228-31); translocation t(14; 18)(q32; q21) involving the bc12 gene and associated with follicular lymphoma; and translocations juxtaposing the coding regions of the PAX3 gene on chromosome 2 and the FKHR gene on chromosome 13 associated with alveolar rhabdomyosarcoma (see Barr F. G. et al. (1996) Hum. Mol. Genet. 5:15-21).

For applications in environmental and industrial diagnostics the capture agents are designed such that they bind to one or more PET corresponding to a biowarfare agent (e.g., anthrax, small pox, cholera toxin) and/or one or more PET corresponding to other environmental toxins (Staphylococcus aureus a-toxin, Shiga toxin, cytotoxic necrotizing factor type 1, Escherichia coli heat-stable toxin, and botulinum and tetanus neurotoxins) or allergens. The capture agents may also be designed to bind to one or more PET corresponding to an infectious agent such as a bacterium, a prion, a parasite, or a PET corresponding to a virus (e.g., human immunodeficiency virus-1 (HIV-1), HIV-2, simian immunodeficiency virus (SIV), hepatitis C virus (HCV), hepatitis B virus (HBV), Influenza, Foot and Mouth Disease virus, and Ebola virus).

The following part illustrates the general idea of diagnostic use of the instant invention in one specific setting—serum biomarker assays.

The proteins found in human plasma perform many important functions in the body. Over or under expression of these proteins can thus cause disease directly, or reveal its presence. Studies have shown that complex serum proteomic patterns might reflect the underlying pathological state of an organ such as the ovary (Petricoin et al., Lancet 359: 572-577, 2002). Therefore, the easy accessibility of serum samples, and the fact that serum comprehensively samples the human phenotype—the state of the body at a particular point in time—make serum an attractive option for a broad array of applications, including clinical and diagnostics applications (early detection and diagnosis of disease, monitor disease progression, monitor therapy etc.), discovery applications (such as novel biomarker discovery), and drug development (drug efficacy and toxicity, and personalized medicine). In fact, over $1 billion annually is spent on immunoassays to measure proteins in plasma as indicators of disease (Plasma Proteome Institute (PPI), Washington, D.C.).

Despite decades of research, only a handful of proteins (about 20) among the 500 or so detected proteins in plasma are measured routinely for diagnostic purposes. These include: cardiac proteins (troponins, myoglobin, creatine kinase) as indicators of heart attack; insulin, for management of diabetes; liver enzymes (alanine or aspartate transaminases) as indicators of drug toxicity; and coagulation factors for management of clotting disorders. About 150 proteins in plasma are measured by some laboratory for diagnosis of less common diseases.

IN addition, proteins in plasma differ in concentration by at least one billion-fold. For example, serum albumin has a normal concentration range of 35-50 mg/mL (35-50×10⁹ pg/mL) and is measured clinically as an indication of severe liver disease or malnutrition, while interleukin 6 (IL-6) has a normal range of just 0-5 pg/mL, and is measured as a sensitive indicator of inflammation or infection.

Thus, there is a need for reference levels of all serum proteins, and reliable assays for measuring serum protein levels under any conditions. However, standardization of immunoassays for heterogeneous antigens is nearly impossible about 10 years ago (Ekins, Scand J Clin Lab Invest. 205: 33-46, 1991). One of the major obstacle is the apparent need of having identical standard and analyte. This is the case with only a few small peptides. With larger peptides and proteins, the problems tend to become more complicated because biological samples often contain proforms, splice variants, fragments, and complexes of the analyte (Stenman, Clinical Chemistry 47: 815-820, 2001). One such problem is illustrated by measuring serum TGF-beta levels.

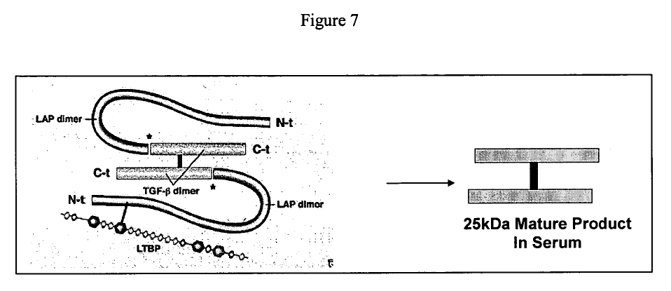

The TGF-beta superfamily proteins are a collection of structurally related multi-function proteins that have a diverse array of biological functions including wound healing, development, oncogenesis, and atherosclerosis. There are at least three known mammalian TGF-beta proteins (beta1, beta2 and beta3), which are thought to have similar functions, at least in vitro. Each of the three isoforms are produced as pre-pro-proteins, which rapidly dimerizes. After the loss of the signal sequences, sugar moieties are added to the proproteins regions known as the Latency Associated Peptide, or LAP. In addition, there is proteolytic cleavage between the LAPs and the mature dimers (the functional portion), but the cleaved LAPs still associate with the mature dimer, forming a complex known as the small latent complex. Either prior to secretion, or in the extracellular milieu, the small latent complex can bind to a large number of other proteins forming a large number of higher molecular weight latent complexes. The best characterized of these proteins are the latent TGF-beta binding protein family LTBP1-4 and fibrillin-1 and -2 (see FIG. 7). Once in the extracellular environment, the TGF-beta complex may bind even more proteins to form other complexes. Known soluble TGF-beta binding proteins include: decorin, alpha-fetoprotein (AFP), betaglycan extracellular domain, β-amyloid precursor, and fetuin. Given the various isoforms, complexes, processing stages, etc., it is very difficult to accurately measure serum TGF-beta protein levels, and a range of 100-fold differences in serum level of TBG-beta1 are reported by different groups (see Grainger et al., Cytokine & Growth Factor Reviews 11: 133-145, 2000).

The other problem arises from the false positive/negative effects of anti-animal antibodies on immunoassays. Specifically, in a sandwich-type assay for a specific antigen in a serum sample, instead of capturing the desired antigen, the immobilized capture antibody may bind to anti-animal antibodies in the serum sample, which in turn can be bound by the labeled secondary antibody and gives rise to false positive result. On the other hand, too much anti-animal antibodies may block the interaction between the capture antibody and the desired antigen, and the interaction between the labeled secondary antibody and the desired antigen, leading to false negative result. This is a serious problem demonstrated in a recent study by Rotmensch and Cole (Lancet 355: 712-715, 2000), which shows that in all 12 cases where women were diagnosed of having postgestational choriocarcinoma on the basis of persistently positive human chorionic gonadotropin (hCG) test results in the absence of pregnancy, a false diagnosis had been made, and most of the women had been subjected to needless surgery or chemotherapy. Such diagnostic problems associated with anti-animal antibodies have also been reported elsewhere (Hennig et al., The influence of naturally occurring heterophilic anti-immunoglobulin antibodies on direct measurement of serum proteins using sandwich ELISAs. Journal of Immunological Methods 235: 71-80, 2000; Covinsky et al., An IgMl Antibody to Escherichia coli Produces False-Positive Results in Multiple Immunometric Assays. Clinical Chemistry 46: 1157-1161, 2000).

All these problems can be efficiently solved by the methods of the instant invention. By digesting serum samples and converting all forms of the target protein to a uniform PET-containing peptide, the methods of the instant invention greatly reduce the complexity of the sample. Anti-animal antibodies, proteins complexes, various isoforms are no longer expected to be a significant factor in the digested serum sample, thus facilitating more reliable, reproducible, and accurate results from assay to assay.

The method of the instant invention is by no means limited to one particular serum protein such as TGF-beta. It has broad applications in a wide range of serum proteins, including peptide hormones, candidate disease biomarkers (such as PSA, CA125, MMPs, etc.), serum disease and non-disease biomarkers, and acute phase response proteins. For example, measuring the following types of serum biomarkers will have broad applications in clinical and diagnostic uses: 1) disease state markers (such as markers for inflammation, infection, etc.), and 2) non-disease state markers, including markers indicating drug and hormone effects (e.g., alcohol, androgens, anti-epileptics, estrogen, pregnancy, hormone replacement therapy, etc.). Exemplary serum proteins that can be measured include: ApoA-I, Andogens, AAT, AAG, A2M, A1b, Apo-B, AT III, C3, Cp, C4, CRP, SAA, Hp, AGP, Fb, AP, FIB, FER, PAL, PSM, Tf, IgA, IgG, IgM, IgE, FN, B2M, and RBP.

One preferred assay method for these serum proteins is the sandwich assay using a PET-specific capture agent and at least one labeled secondary capture agent(s) for detection of binding. These assays may be performed in an array format according to the teaching of the instant application, in that different capture agents (such as PET-specific antibodies) can be arrayed on a single (or a few) microarrays for use in simultaneous detection/quantitation of a large number of serum biomarkers.

Foundation for Blood Research (FBR, Scarborough, Me.) has developed a 152-page guide on serum protein utility and interpretation for day to day use by practitioners and laboratorians. This guide contains a distillation of the world's literature on the subject, is fully indexed, and is presented by a given disease state (Section I), as well as by individual proteins (Section II). This book is generally useful for interpretation of test results, as well as providing guidance regarding which test is (or is not) appropriate to order and why (or why not). Section II, which covers general information on serum proteins, is also helpful regarding background information about each protein. The entire content of which is incorporated herein by reference.

B. High-Throughput Screening

Compositions containing the capture agents of the invention, e.g., microarrays, beads or chips enable the high-throughput screening of very large numbers of compounds to identify those compounds capable of interacting with a particular capture agent, or to detect molecules which compete for binding with the PETs. Microarrays are useful for screening large libraries of natural or synthetic compounds to identify competitors of natural or non-natural ligands for the capture agent, which may be of diagnostic, prognostic, therapeutic or scientific interest.

The use of microarray technology with the capture agents of the present invention enables comprehensive profiling of large numbers of proteins from normal and diseased-state serum, cells, and tissues.

For example, once the microarray has been formed, it may be used for high-throughput drug discovery (e.g., screening libraries of compounds for their ability to bind to or modulate the activity of a target protein); for high-throughput target identification (e.g., correlating a protein with a disease process); for high-throughput target validation (e.g., manipulating a protein by, for example, mutagenesis and monitoring the effects of the manipulation on the protein or on other proteins); or in basic research (e.g., to study patterns of protein expression at, for example, key developmental or cell cycle time points or to study patterns of protein expression in response to various stimuli).

In one embodiment, the invention provides a method for identifying a test compound, e.g., a small molecule, that modulates the activity of a ligand of interest. According to this embodiment, a capture agent is exposed to a ligand and a test compound. The presence or the absence of binding between the capture agent and the ligand is then detected to determine the modulatory effect of the test compound on the ligand. In a preferred embodiment, a microarray of capture agents, that bind to ligands acting in the same cellular pathway, are used to profile the regulatory effect of a test compound on all these proteins in a parallel fashion.

C. Pharmacoproteomics

The capture agents or arrays comprising the capture agents of the present invention may also be used to study the relationship between a subject's protein expression profile and that subject's response to a foreign compound or drug. Differences in metabolism of therapeutics can lead to severe toxicity or therapeutic failure by altering the relation between dose and blood concentration of the pharmacologically active drug. Thus, use of the capture agents in the foregoing manner may aid a physician or clinician in determining whether to administer a pharmacologically active drug to a subject, as well as in tailoring the dosage and/or therapeutic regimen of treatment with the drug.

D. Protein Profiling

As indicated above, capture agents of the present invention enable the characterization of any biological state via protein profiling. The term “protein profile,” as used herein, includes the pattern of protein expression obtained for a given tissue or cell under a given set of conditions. Such conditions may include, but are not limited to, cellular growth, apoptosis, proliferation, differentiation, transformation, tumorigenesis, metastasis, and carcinogen exposure.

The capture agents of the present invention may also be used to compare the protein expression patterns of two cells or different populations of cells. Methods of comparing the protein expression of two cells or populations of cells are particularly useful for the understanding of biological processes. For example, using these methods, the protein expression patterns of identical cells or closely related cells exposed to different conditions can be compared. Most typically, the protein content of one cell or population of cells is compared to the protein content of a control cell or population of cells. As indicated above, one of the cells or populations of cells may be neoplastic and the other cell is not. In another embodiment, one of the two cells or populations of cells being assayed may be infected with a pathogen. Alternatively, one of the two cells or populations of cells has been exposed to a chemical, environmental, or thermal stress and the other cell or population of cells serves as a control. In a further embodiment, one of the cells or populations of cells may be exposed to a drug or a potential drug and its protein expression pattern compared to a control cell.

Such methods of assaying differential protein expression are useful in the identification and validation of new potential drug targets as well as for drug screening. For instance, the capture agents and the methods of the invention may be used to identify a protein which is overexpressed in tumor cells, but not in normal cells. This protein may be a target for drug intervention. Inhibitors to the action of the overexpressed protein can then be developed. Alternatively, antisense strategies to inhibit the overexpression may be developed. In another instance, the protein expression pattern of a cell, or population of cells, which has been exposed to a drug or potential drug can be compared to that of a cell, or population of cells, which has not been exposed to the drug. This comparison will provide insight as to whether the drug has had the desired effect on a target protein (drug efficacy) and whether other proteins of the cell, or population of cells, have also been affected (drug specificity).

E. Protein Sequencing, Purification and Characterization

The capture agents of the present invention may also be used in protein sequencing. Briefly, capture agents are raised that interact with a known combination of unique recognition sequences. Subsequently, a protein of interest is fragmented using the methods described herein to generate a collection of peptides and then the sample is allowed to interact with the capture agents. Based on the interaction pattern between the collection of peptides and the capture agents, the amino acid sequence of the collection of peptides may be deciphered. In a preferred embodiment, the capture agents are immobilized on an array in pre-determined positions that allow for easy determination of peptide-capture agent interactions. These sequencing methods would further allow the identification of amino acid polymorphisms, e.g., single amino acid polymorphisms, or mutations in a protein of interest.

In another embodiment, the capture agents of the present invention may also be used in protein purification. In this embodiment, the PET acts as a ligand/affinity tag and allows for affinity purification of a protein. A capture agent raised against a PET exposed on a surface of a protein may be coupled to a column of interest using art known techniques. The choice of a column will depend on the amino acid sequence of the capture agent and which end will be linked to the matrix. For example, if the amino-terminal end of the capture agent is to be linked to the matrix, matrices such as the Affigel (by Biorad) may be used. If a linkage via a cysteine residue is desired, an Epoxy-Sepharose-6B column (by Pharmacia) may be used. A sample containing the protein of interest may then be run through the column and the protein of interest may be eluted using art known techniques as described in, for example, J. Nilsson et al. (1997) “Affinity fusion strategies for detection, purification, and immobilization of recombinant proteins,” Protein Expression and Purification, 11:11-16, the contents of which are incorporated by reference. This embodiment of the invention also allows for the characterization of protein-protein interactions under native conditions, without the need to introduce artificial affinity tags in the protein(s) to be studied.

In yet another embodiment, the capture agents of the present invention may be used in protein characterization. Capture agents can be generated that differentiate between alternative forms of the same gene product, e.g., between proteins having different post-translational modifications (e.g., phosphorylated versus non-phosphorylated versions of the same protein or glycosylated versus non-glycosylated versions of the same protein) or between alternatively spliced gene products.

The utility of the invention is not limited to diagnosis. The system and methods described herein may also be useful for screening, making prognosis of disease outcomes, and providing treatment modality suggestion based on the profiling of the pathologic cells, prognosis of the outcome of a normal lesion and susceptibility of lesions to malignant transformation.

F. Detection of Post-Translational Modifications

The subject computer generated PETs can also be analyzed according to the likely presence or absence of post-translational modifications. More than 100 different such modifications of amino acid residues are known, examples include but are not limited to acetylation, amidation, deamidation, prenylation (such as farnesylation or geranylation), formylation, glycosylation, hydroxylation, methylation, myristoylation, phosphorylation, ubiquitination, ribosylation and sulphation. Sequence analysis softwares which are capable of determining putative post-translational modification in a given amino acid sequence include the NetPhos server which produces neural network predictions for serine, threonine and tyrosine phosphorylation sites in eukaryotic proteins (available through http://www.cbs.dtu.dk/services/Net-Phos/), GPI Modification Site Prediction (available through http://mendel.imp.univie.ac.at/gpi) and the ExPASy proteomics server for total protein analysis (available through www.expasy.ch/tools/)

In certain embodiments, preferred PET moieties are those lacking any post-translational modification sites, since post-translationally modified amino acid sequences may complicate sample preparation and/or interaction with a capture agent. Notwithstanding the above, capture agents that can discriminate between post-translationally forms of a PET, which may indicate a biological activity of the polypeptide-of-interest, can be generated and used in the present invention. A very common example is the phosphorylation of OH group of the amino acid side chain of a serine, a threonine, or a tyrosine group in a polypeptide. Depending on the polypeptide, this modification can increase or decrease its functional activity. In one embodiment, the subject invention provides an array of capture agents that are variegated so as to provide discriminatory binding and identification of various post-translationally modified forms of one or more proteins. In a preferred alternative embodiment, the subject invention provides an array of capture agents that are variegated so as to provide specific binding to one or more PET uniquely associated with a modification of interest, which modification itself can be readily detected and/or quantitated by additional agents, such as a labeled secondary antibody specifically recognizing the modification (e.g., a phospho-tyrosine antibody).

In a general sense, the invention provides a general means to detect/quantitate protein modifications. “Modification” here refers generally to any kind of non-wildtype changes in amino acid sequence, including post-translational modification, alternative splicing, polymorphysm, insertion, deletion, point mutation, etc. To detect/quantitate a specific modification within a potential target protein present in a sample, the sequence of the target protein is first analyzed to identify potential modification sites (such as phosphorylation sites for a specific kinase). Next, a potential fragment of the target protein containing such modification site is identified. The fragment is specific for a selected method of treatment, such as tryptic digestion or digestion by another protease or reliable chemical fragmentation. PET within (and unique) to the modification site-containing fragment can then be identified using the method of the instant invention. Fragmentation using a combination of two or more methods is also contemplated. Absolute predictability of the fragment size is desired, but not necessary, as long as the fragment always contains the desired PET and the modification site.

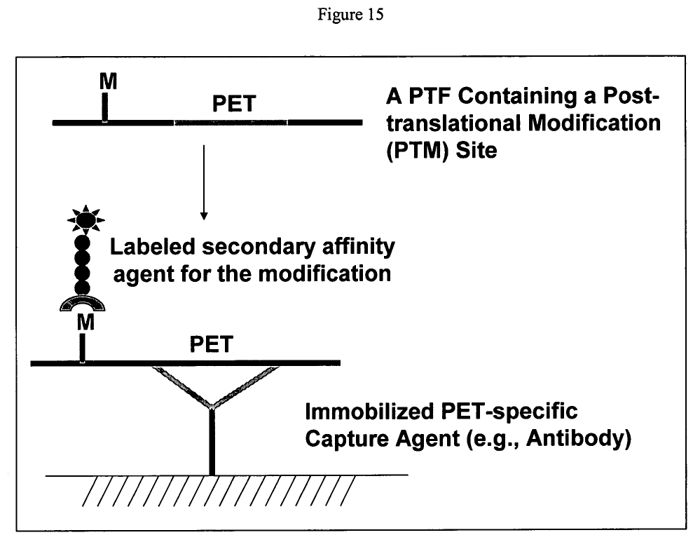

Antibody or other capture agents specific for the identified PET is obtained. The capture agent is then used in a sandwich ELISA format to detect captured fragments containing the modification (see FIG. 15). The site of the PET is proximal to the post-translational modification site(s). Thus a binding to the PET by a capture agent will not interfere with the binding of a detection agent specific for the modified residue.

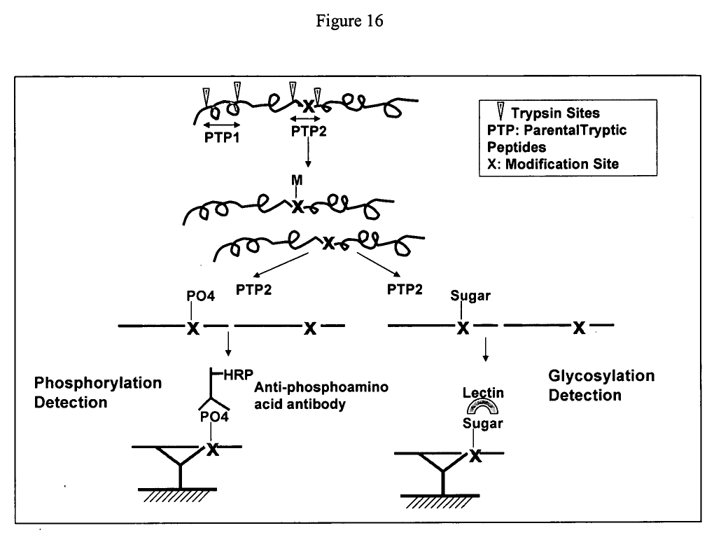

A few specific embodiments of this aspect of the invention are described in more detail below (see FIG. 16). For illustrative purpose only, the capture agents described below in various embodiments of the invention are antibodies specific for PETs. However, it should be understood that any capture agents described above can be used in each of the following embodiments.

(i) Phosphorylation

The reversible addition of phosphate groups to proteins is important for the transmission of signals within eukaryotic cells and, as a result, protein phosphorylation and dephosphorylation regulate many diverse cellular processes. To detect the presence and/or quantitate the amount of a phosphorylated peptide in a sample, anti-phospho-amino acid antibodies can be used to detect the presence of phosphopeptides.

There are numerous commercially available phospho-tyrosine specific antibodies that can be adapted to be used in the instant invention. Merely to illustrate, phosphotyrosine antibody (ab2287) [13F9] of Abcam Ltd (Cambridge, UK) is a mouse IgG1 isotype monoclonal antibody reacts specifically with phosphotyrosine and shows minimal reactivity by ELISA and competitive ELISA with phosphoserine or phosphothreonine. The antibody reacts with free phosphotyrosine, phosphotyrosine conjugated to carriers such as thyroglobulin or BSA, and detects the presence of phosphotyrosine in proteins of both unstimulated and stimulated cell lysates.

Similarly, RESEARCH DIAGNOSTICS INC (Flanders, N.J.) provides a few similar anti-phosphotyrosine antibodies. Among them, RDI-PHOSTYRabmb is a mouse mIgG2b isotype monoclonal antibody reacts strongly and specifically with phosphotyrosine-containing proteins and can be blocked specifically with phosphotyrosine. No reaction with either phosphothreonine or phosphoserine is detected. This antibody appears to have broad cross-species reactivity, and is reactive with various tyrosine-phosphorylated proteins of human, chick, frog, rat, mouse and dog origin.

RESEARCH DIAGNOSTICS INC also provides phosphoserine-specific antibodies, such as RDI-PHOSSERabr, which is an affinity-purified rabbit antibody made against phosphoserine containing proteins. The antibody reacts specifically with serine phosphorylated proteins and shows no significant cross reactivity to other phosphothreonine or phosphotyrosine by western blot analysis. This antibody is suitable for ELISA according to the manufacture's suggestion. The company also provides a mouse IgG1 monoclonal anti-phosphoserine antibody RDI-PHOSSEabm, which reacts specifically with phosphorylated serine, both as free amino acid or conjugated to carriers as BSA or KLH. No cross reactivity is observed with non-phosphorylated serine, phosphothreonine, phosphotyrosine, AmpMP or ATP.

RDI-PHOSTHRabr is an affinity isolated rabbit anti-phosphothreonine antibody (anti-pT) provided by RESEARCH DIAGNOSTICS INC. Both antigen-capture and antibody-capture ELISA indicated that the anti-phosphothreonine antibodies can recognize threonine-phosphorylated protein, phosphothreonine and lysine-phosphothreonine-glycine random polymer, respectively. Direct, competitive antigen-capture ELISA demonstrated that the antibodies are specifically inhibited by free phosphothreonine, phosvitin but not by free phosphoserine, phosphotyrosine, threonine and ATP. The company also provides a mouse IgG2b monoclonal anti-phosphothreonine antibody RDI-PHOSTHabm, which reacts specifically with phosphorylated threonine, both as free amino acid or conjugated to carriers as BSA or KLH. No cross reactivity is observed with non-phosphorylated threonine, phophoserine, phosphotyrosine, AmpMP or ATP.

Molecular Probe (Eugene, Oreg.) has developed a small molecule fluorophore phosphosensor, referred to as Pro-Q Diamond dye, which is capable of ultrasensitive global detection and quantitation of phosphorylated amino acid residues in peptides and proteins displayed on microarrays. The utility of the fluorescent Pro-Q Diamond phosphosensor dye technology is demonstrated using phosphoproteins and phosphopeptides as well as with protein kinase reactions performed in miniaturized microarray assay format (Martin, et al., Proteomics 3: 1244-1255, 2003). Instead of applying a phosphoamino acid-selective antibody labeled with a fluorescent or enzymatic tag for detection, a small, fluorescent probe is employed as a universal sensor of phosphorylation status. The detection limit for phosphoproteins on a variety of different commercially available protein array substrates was found to be 312-625 fg, depending upon the number of phosphate residues. Characterization of the enzymatic phosphorylation of immobilized peptide targets with Pro-Q Diamond dye readily permits differentiation between specific and non-specific peptide labeling at picogram to subpicogram levels of detection sensitivity. Martin et al. (supra) also describe in detail the suitable protocols, instruments for using the Pro-Q stain, especially for peptides on microarrays, the entire contents of which are incorporated herein by reference.

One of the advantageous of the method over other methods, such as identification of modified amino acids in proteins by mass spectrometry, is that the instant invention provides a much simpler technique that does not rely on expensive instruments, and thus can be easily adapted to be used in small or large laboratories, in industry or academic settings alike.

In one embodiment, the instant invention can be used to identify potential substrates of a specific kinase or kinase subfamily. As the number of known protein kinases has increased at an ever-accelerating pace, it has become more challenging to determine which protein kinases interact with which substrates in the cell.

The determination of consensus phosphorylation site motifs by amino acid sequence alignment of known substrates has proven useful in this pursuit. These motifs can be helpful for predicting phosphorylation sites for specific protein kinases within a potential protein substrate. The table below summarizes merely some of the known data about specificity motifs for various well-studied protein kinases, along with examples of known phosphorylation sites in specific proteins (for a more extensive list, see Pearson, R. B., and Kemp, B. E. (1991). In T. Hunter and B. M. Sefton (Eds.), Methods in Enzymology Vol. 200, pp. 62-81. San Diego: Academic Press, incorporated by reference). Phosphoacceptor residue is indicated in bold, amino acids which can function interchangeably at a particular residue are separated by a slash (/), and residues which do not appear to contribute strongly to recognition are indicated by an “X.” Some protein kinases such as CKI and GSK-3 contain phosphoamino acid residues in their recognition motifs, and have been termed “hierarchical” protein kinases (see Roach, J. Biol. Chem. 266, 14139-14142, 1991 for review). They often require prior phosphorylation by another kinase at a residue in the vicinity of their own phosphorylation site. S(p) represents such preexisting phosphoserine residues.

| | Recognition | Phosphorylation | Protein Substrate |
|---|---|---|---|
| Protein Kinase | Motifs^{a} | Sites_{b} | (reference) |
| cAMP-dependent | R-X-S/T^{c} | Y₇LRRASLAQLT | pyruvate kinase (2) |
| Protein Kinase | R-R/K-X-S/T | F₁RRLSIST | phosphorylase kinase, |
| (PKA, cAPK) | | A₂₉GARRKASGPP | a chain (2) |
| | | | histone H1, bovine (2) |
| Casein Kinase I | S(P)-X-X-S/T | R₄TLS(P)VSSLPGL | glycogen synthase, |
| (CKI, CK-1) | | D₄₃IGS(p)ES(p)TEDQ | rabbit muscle (4) |
| | | | aₛ₁-casein (4) |
| Casein Kinase II | S/T-X-X-E | A₇₂DSESEDEED | PKA regulatory |
| (CKII, CK-2) | | L₃₇ESEEEGVPST | subunit, R_{II}(2) |
| | | E₂₆DNSEDEISNL | p34^{cdc2}, human (5) |
| | | | acetyl-CoA |
| | | | carboxylase (2) |
| Glycogen Synthase | S-X-X-X-S(p) | S₆₄₁VPPSPSLS(p) | glycogen synthase, |
| Kinase 3 (GSK-3) | | S₆₄₁VPPS(p)PSLS(p) | human (site 3b) (6, 2) |
| | | | glycogen synthase, |
| | | | human (site 3a) (6, 2) |
| Cdc2 Protein | S/T-P-X-R/K^{c} | P₁₃AKTPVK | histone H1, calf |
| Kinase | | H₁₂₂STPPKKKRK | thymus (2) |
| | | | large T antigen (2) |
| Calmodulin- | R-X-X-S/T | N₂YLRRRLSDSN | synapsin (site 1) (2) |
| dependent Protein | R-X-X-S/T-V | K₁₉₁MARVFSVLR | calcineurin (2) |
| Kinase II (CaMK | | | |
| II) | | | |
| Mitogen-activated | P-X-S/T-P^{d} | P₂₄₄LSP | c-Jun (7) |
| Protein Kinase | X-X-S/T-P | P₉₂SSP | cyclin B (7) |
| (Extracellular | | V₄₂₀LSP | Elk-1 (7) |
| Signal-regulated | | | |
| Kinase) (MAPK, | | | |
| Erk) | | | |
| cGMP-dependent | R/K-X-S/T | G₂₆KKRKRSRKES | histone H2B (2) |
| Protein Kinase | R/K-X-X-S/T | F₁RRLSIST | phosphorylase kinase |
| (cGPK) | | | (a chain) (2) |
| Phosphorylase | K/R-X-X-S-V/I | D₆QEKRKQISVRG | phosphorylase (2) |
| Kinase (PhK) | | P₁LSRTLSVSS | glycogen synthase |
| | | | (site 2) (2) |
| Protein Kinase C | S/T-X-K/R | H₅₉₄EGTHSTKR | fibrinogen (2) |
| (PKC) | K/R-X-X-S/T | P₁LSRTLSVSS | glycogen synthase |
| | K/R-X-S/T | Q₄KRPSQRSKYL | (site 2) (2) |
| | | | myelin basic protein |
| | | | (2) |
| Abl Tyrosine | I/V/L-Y-X-X-P/F^{e} | | |
| Kinase | | | |
| Epidermal Growth | E/D-Y-X | R₁₁₆₈ENAEYLRVAP | autophosphorylation |
| Factor Receptor | E/D-Y-I/L/V | A₇₆₇EPDYGALYE | (2) |
| Kinase (EGF-RK) | | | phospholipase C-g(2) |
| Single-letter Amino Acid Code: | | | |
| A = alanine, C = cysteine, D = aspartic acid, E = glutamic acid, F = phenylalanine, G = glycine, H = histidine, I = isoleucine, K = lysine, L = leucine, M = methionine, N = asparagine, P = proline, Q = glutamine, R = arginine, S = serine, T = threonine, W = tryptophan, | | | |
| # V = valine, Y = tyrosine, X = any amino acid | | | |

^{a}Recognition motifs are taken from Pearson and Kemp (supra) except where noted. Consult Pearson and Kemp for a comprehensive list of phosphorylation site sequences and specificity motifs.
^{b}Subscripted numbers refer to the position of the first residue within the given polypeptide chain.
^{c}From (1).
^{d}From (7).
^{e}From (8). See refs (8) and (9) for discussion of substrate recognition by Abl.
References used in the table above:
1. Kennelly, P. J., and Krebs, E. G. (1991) J. Biol. Chem. 266, 15555-15558.
2. Pearson, R. B., and Kemp, B. E. (1991). In T. Hunter and B. M. Sefton (Eds.), Methods in Enzymology Vol. 200, (pp. 62-81). San Diego: Academic Press.
3. Roach, P. J. (1991) J. Biol. Chem. 266, 14139-14142.
4. Flotow, H. et al. (1990) J. Biol. Chem. 265, 14264-14269.
5. Russo, G. L. et al. (1992) J. Biol. Chem. 267, 20317-20325.
6. Fiol, C. J. et al. (1990) J. Biol. Chem. 265, 6061-6065.
7. Davis, R. J. (1993) J. Biol. Chem. 268, 14553-14556.
8. Songyang, Z. et al. (1995) Nature 373, 536-539.
9. Geahlen, R. L. and Harrison, M. L. (1990). In B. E. Kemp (Ed.), Peptides and Protein Phosphorylation, (pp. 239-253). Boca Raton: CRC Press.

However, since the determinants of protein kinase specificity involve complex 3-dimensional interactions, these motifs, short amino-acid sequences describing the primary structure around the phosphoacceptor residue, are a significant oversimplification of the issue. They do not take into account possible secondary and tertiary structural elements, or determinants from other polypeptide chains or from distant locations within the same chain. Furthermore, not all of the residues described in a particular specificity motif may carry the same weight in determining recognition and phosphorylation by the kinase. In addition, the potential recognition sequence may be buried deep inside a tertiary structure of within a protein complex under physiological conditions and thus may never be accessible in vivo. As a consequence, they should be used with some caution. The instant invention provides a fast and convenient way to determine, on a proteome-wide basis, the identity of all potential kinase substrates that actually do become phopshorylated by the kinase of interest in vivo (or in vitro).

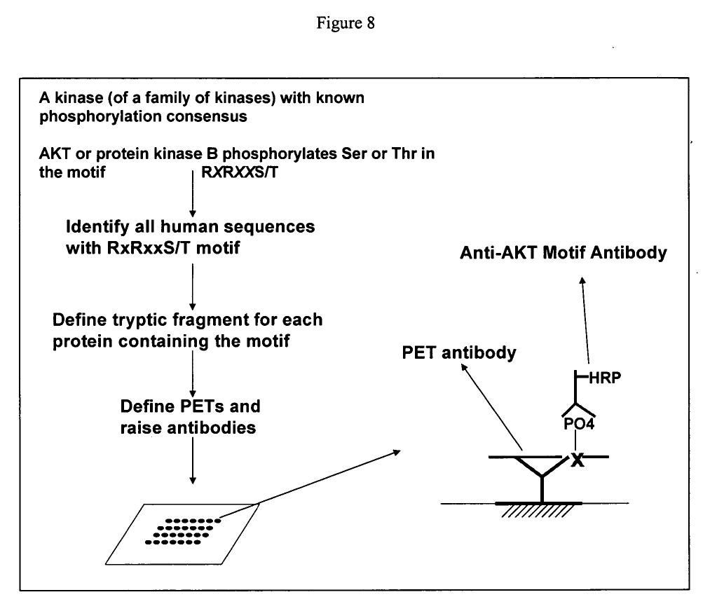

Specifically, consensus recognition sequences of a kinase (or a kinase subfamily sharing substrate specificity) can be identified based on, for example, Pearson and Kemp or other kinase substrate motif database. For example, AKT (or PKB) kinase has a consensus phosphorylation site sequence of RXRXXS/T. All proteins in an organism (e.g., human) that contains this potential recognition sequence can be readily identified through routine sequence searches. Using the method of the instant invention, peptide fragments of these potential substrates, after a pre-determined treatment (such as trypsin digestion), which contain both the recognition motif and at least one PET can then be generated. Antibodies (or other capture agents) against each of these identified PETs can be raised and printed on an array to generate a so-called “kinase chip,” in this case, an AKT chip. Using this chip, any sample to be studied can be treated as described above and then be incubated with the chip so that all potential recognition site-containing fragments are captured. The presence or absence of phosphorylation on any given “spot”—a specific potential substrate—can be detected/quantitated by, for example, labeled secondary antibodies (see FIG. 8). Thus, the identity of all AKT substrates in this organism under this condition may be identified in one experiment. The array can be reused for other samples by eluting the bound peptides on the array. Different arrays can be used in combination, preferably in the same experiment, to determine the substrates for multiple kinases.

The reversible phosphorylation of tyrosine residues is an important mechanism for modulating biological processes such as cellular signaling, differentiation, and growth, and if deregulated, can result in various types of cancer. Therefore, an understanding of these dynamic cellular processes at the molecular level requires the ability to assess changes in the sites of tyrosine phosphorylation across numerous proteins simultaneously as well as over time. Thus in another embodiment, the instant invention provides a method to identify the various signal transduction pathways activated after a specific treatment to a sample, such as before and after a specific growth factor or cytokine treatment to a sample cell. The same method can also be used to compare the status of signal transduction pathways in a diseased sample from a patient and a normal sample from the same patient.

Know ledges about the various signal transduction pathways existing in various organisms are accumulating at an astonishing pace. Science magazine's STKE (Signal Transduction Knowledge Environment) maintains a comprehensive and expanding list of known signal transduction pathways, their important components, relationship between the components (inhibit, stimulation, etc.), and cross-talk between key members of the different pathways. The “Connections Map” provides a dynamic graphical interface into a cellular signaling database, which currently covers at least the following broad pathways: immune pathways (IL-4, IL-13, Token-like receptor); seven-transmembrane receptor pathways (Adrenergic, PAC1 receptor, Dictyostelium discoideum cAMP Chemotaxis, Wnt/Ca²⁺/cyclic GMP, G Protein-Independent 7 Transmembrane Receptor); Circadian Rhythm pathway (murine and Drosophila); Insulin pathway; FAS pathway; TNF pathway; G-Protein Coupled Receptor pathways; Integrin pathways; Mitogen-Activated Protein Kinase Pathways (MAPK, JNK, p38); Estrogen Receptor Pathway; Phosphoinositide 3-Kinase Pathway; Transforming Growth Factor-β (TGF-β) Pathway; B Cell Antigen Receptor Pathway; Jak-STAT Pathway; STAT3 Pathway; T Cell Signal Transduction Pathway; Type 1 Interferon (α/β) Pathway; Jasmonate Biochemical Pathway; and Jasmonate Signaling Pathway. Many other well-known signal transduction pathways not yet included are described in detail in other scientific literatures which can be readily identified in PubMed or other common search tools. Activation of most, if not all of these signal transduction pathways are generally characterized by changes in phosphorylation levels of one or more members of each pathway.

Thus in a general sense, the status of any given number of signaling pathways in a sample can be determined by taking a “snap shot” of the phosphorylation status of one or more key members of these selected pathways. For example, the Mitogen-activated protein (MAP)1 kinase pathways are evolutionarily conserved in eukaryotic cells. The pathways are essential for physiological processes, such as embryonic development and immune response, and regulate cell survival, apoptosis, proliferation, differentiation, and migration. In mammals, three major classes of MAP kinases (MAPKS) have been identified, which differ in their substrate specificity and regulation. These subgroups compose the extracellular signal-regulated kinases (ERKs), the c-Jun N-terminal kinases (JNKs), and the p38/RK/CSBP kinases. ERKs are activated by a range of stimuli including growth factors, cell adhesion, tumor-promoting phorbol esters, and oncogenes, whereas JNK and p38 are preferentially activated by proinflammatory cytokines, and a variety of environmental stresses such as UV and osmotic stress. For this reason, the latter are classified as stress-activated protein kinases. Activation of the MAPKs is achieved by dual phosphorylation on threonine and tyrosine residues within a Thr-Xaa-Tyr motif located in the kinase subdomain VIII. This phosphorylation is mediated by a dual specificity protein kinase, MAPK kinase (MAPKK), and MAPKK is in turn activated by phosphorylation mediated by a serine/threonine protein kinase, MAPKK kinase. In addition to these activating kinases, several types of protein phosphatases have been also shown to control MAPK pathways by dephosphorylating the MAPKs or their upstream kinases. These protein phosphatases include tyrosine-specific phosphatases, serine/threonine-specific phosphatases, and dual specificity phosphatases (DSPs). Therefore, the activities of MAPKs can be regulated by upstream activating kinases and protein phosphatases, and the activation status can be determined by the phosphorylation status of, for example, ERK1/2, JNK, and p38.

Specifically, fragments of ERK1/2, JNK, and p38 containing the signature phosphorylation sites and PETs can be identified using the methods of the instant invention. Capture agents specifically recognizing such phosphorylation site-associated PETs can then be raised and immobilized on an array/chip. A sample (treated or untreated, thus containing high or low levels of phosphorylation of these pathway markers) can be digested and incubated with the chip, so as to determine the presence/absence of activation, and degree, time course, duration of activation, etc.

In the same principal, many other related or perceived unrelated pathways may be manufactured on the same chip, since each pathway may be represented by from just one, to possibly all of the known pathway components. This type of chip may provide a comprehensive view of the various pathways that may be activated after a drug treatment. Pathway specific chips may also be used in conjunction to further determine the status of individual components within a pathway of interest.

Because of the important functions of the kinases in virtually all kinds of signal transduction pathways, it is not surprising to see that many drugs directly or indirectly affects phosphorylation status of carious kinase substrates. Thus this type of array may also be used in drug target identification. Briefly, samples treated by different drug candidates may be incubated with the same kind of array to generate a series of activation profiles of certain chosen targets. These profiles may be compared, preferably automatically, to determine which drug candidate has the same or similar activation profile as that of the lead molecule.

This type of experiment will also yield useful information concerning the selectivity of candidate drugs, since it can be easily determined whether a candidate drug or drug analog actually have differential effects on various pathways, and if so, whether the difference is significant.

The same type of experiments can also be adapted to screen for drug candidates that lacks undesired side effects or toxicity.

One aspect of this type of application relates to the selection of specific protease(s) for fragmentation. The following table presents data resulting from analysis of protease sensitivity of potential phosphorylation sites in the human “kinome” (all kinases). This table may aid the selection of proteases among the several most frequently used proteases.

| | Total | | |
|---|---|---|---|
| | Peptide | Peptide Fragments with S/T/Y | |
| Enzymes | Fragments | =<10 aa | >10 aa |
| Chymotrypsin | 34,094 | 10930 (43%) | 14985 (57%) |
| S.A. V-8 E specific Enzyme | 34,233 | 6753 (32%) | 14917 (68%) |
| Post-Proline Cleaving Enzyme | 29,715 | 7077 (37%) | 12224 (63%) |
| Trypsin | 54,260 | 15,217 (53%) | 13311 (47%) |

A wide variety of eukaryotic membrane-bound and secreted proteins are glycosylated, that is they contain covalently-bound carbohydrate, and therefore are termed glycoproteins. In addition, certain intracellular eukaryotic proteins are also glycoproteins. Glycosylation of polypeptides in eukaryotes occurs principally in three ways (Parekh et al., Trends Biotechnol. 7: 117, 1989). Glycosylation through a glycosidic bond to an asparagine side-chain is known as N-glycosylation. Such asparagine residues only occur in the amino acid triplet sequence of Asn-Xaa-Ser/Thr, where Xaa can be any amino acid. The carbohydrate portion of a glycoprotein is also known as a glycan. O-glycans are linked to serine or threonine side-chains, through O-glycosidic bonds. In human, 284,535 octamer tags contains this NX(S/T) sequence, and 228,256 octamer PETs contains the NX(S/T) sequence. The latter is about 2.6% of the total octamer peptide tags in human. The N- and O-linked glycosylation are two of the most complex post-translational modifications. The polypeptide may also be linked to a phosphatidylinositol lipid anchor through a carbohydrate “bridge”, the whole assembly being known as the glycosyl-phosphatidylinositol (GPI) anchor.

In recent years, the functional significance of the carbohydrate moieties has been increasingly appreciated (Rademacher et al., Ann. Rev. Biochem. 57: 785, 1988). Carbohydrates covalently attached to polypeptide chains can confer many functions to the glycoprotein, for example resistance to proteolytic degradation, the transduction of information between cells, and intercellular adhesion through ligand-receptor interactions (Gesundheit et al., J. Biol. Chem. 262: 5197, 1987; Ashwell & Harford, Ann. Rev. Biochem. 51: 531, 1982; Podskalny et al., J. Biol. Chem. 261: 14076, 1986; Dennis et al., Science 236: 582, 1987). As glycoforms are the product of a series of biochemical modifications, perturbations within a cell can have profound effects on their structure. With the increase in understanding of carbohydrate functions, the need for rapid, reliable and sensitive methods for carbohydrate detection and analysis has grown considerably.

Lectins are proteins that interact specifically and reversibly with certain sugar residues. Their specificity enables binding to polysaccharides and glycoproteins (even agglutination of erythrocytes and tumor cells). The binding reaction between a lectin and a specific sugar residue is analogous to the interaction between an antibody and an antigen. Substances bound to lectin may be resolved with a competitive binding substance or an ionic strength gradient. In addition, among other procedures, lectins can be labeled with biotin or digoxigenin, and subsequently detected by avidin-conjugated peroxidase or anti-digoxigenin antibodies coupled with alkaline phosphatase, respectively (Carlsson S R: Isolation and characterization of glycoproteins. In: Glycobiology. A Practical Approach. Fukuda M and Kobata A (eds). Oxford University Press, Oxford, pp 1-25, 1993, incorporated herein by reference).

For example, Concanavalin A (Con A) binds molecules that contain α-D-mannose, α-D-glucose and sterically related residues with available C-3, C4, or C-5 hydroxyl groups. Like Con A, lentil lectin binds α-D-mannose, α-D-glucose, and sterically related residues, but lentil lectin distinguishes less sharply between glucosyl and mannosyl residues and binds simple sugars with lower affinity. Agarose wheat germ lectin specifically binds to N-acetyl-β-glucosaminyl residues. Wheat germ lectin specifically binds to N-acetyl-β-D-glucosaminyl residues. Psathyrella velutina lectin (PVL) preferentially interacts with the N-acetylglucosamine beta 1—>2Man group. All these lectins can be used to detect the presence of various kinds of glycosylated peptides fragments after these PET-associated glycosylated peptide fragments are captured from the sample by capture agents.

The GlycoTrack Kit from Glyko, Inc. (a Prozyme company, San Leandro, Calif.) detect glycosylation by using a specific carbohydrate oxidation reaction prior to binding of a high amplification color generating reagent. Briefly, a sample, either in solution or already immobilized to a support, is oxidized with periodate. This generates aldehyde groups that can react spontaneously with certain hydrazides at room temperature in aqueous conditions. Use of biotin-hydrazide following periodate oxidation leads to the incorporation of biotin into the carbohydrate (9). The biotinylated compound is detected by reaction with a streptavidin-alkaline phosphatase conjugate. Subsequently visualization is achieved using a substrate that reacts with the alkaline phosphatase bound to glycoproteins on the membrane, forming a colored precipitate.

Molecular Probes (Eugene, Oreg.) offer a proprietary Pro-Q Emerald 300 fluorescent glycoprotein stain for detection of glycoproteins. The new Pro-Q Emerald 300 fluorescent glycoprotein stain reacts with periodate-oxidized carbohydrate groups, creating a bright green-fluorescent signal on glycoproteins. Depending upon the nature and the degree of glycosylation, this stain may be 50-fold more sensitive than the standard periodic acid-Schiff base method using acidic fuchsin dye. According to the manufacture, detection using the Pro-Q Emerald 300 glycoprotein stain is much easier than detection of glycoproteins using biotin hydrazide with streptavidin-horseradish peroxidase and ECL detection (Amersham Pharmacia Biotech). The stain can detect 50 ng of a typical glycosylated protein. Since the captured glycosylated PET-containing peptide fragments are much smaller than a typocal peptide, as little as low nanogram to high picograms of captured peptides can be detected using this dye.

Thus to detect the presence and quantitation of glycosylation in a sample, all proteins or a subpopulation thereof which contains the potential glycosylation site NXS/T may be identified, and peptide fragments resulting from a specific pre-determined treatment may be analyzed to identify associated PETs. Capture agents against these PETs can then be raised. In a method analogous to the phosphorylation detection as described above, glycosylation can be detected/quantitated using the various detection methods

(iii) Other Post-Translational Modifications

Capture agents, such as antibodies specific for other post-translationally modified residues are also readily availble.

There are at least 46 anti-ubiquitin commercial antibodies available from 14 different vendors. For example, Cell Signaling Technology (Beverly, Mass.) offers mouse anti-Ubiquitin monoclonal antibody, clone P4D1 (IgG1 isotype, Cat. No. 3936), which is specific for all species of ubiquitin, polyubiquitin, and ubiquitinated peptides.

Anti-acetylated amino acid antibodies have also been commerciallized. See anti-acetylated-histon H3 and H4 antibodies (Catalog # 06-599 and Catalog # 06-598) from Upstate Biotechnology (Lake Placid, N.Y.). In fact, Alpha Diagnostic International, Inc. (San Antonio, Tex.) offers custom synthesis of anti-acetylated amino acid antibodies.

Arginine methylation, a protein modification discovered almost 30 years ago, has recently experienced a renewed interest as several new arginine methyltransferases have been identified and numerous proteins were found to be regulated by methylation on arginine residues. Mowen and David published detailed protocols on Science's STKE (www.stke.org/cgi/content/full/OC_sigtrans; 2001/93/p11) that provide guidelines for the straightforward identification of arginine-methylated proteins, made possible by the availability of novel, commercially available reagents. Specifically, two anti-methylated arginine antibodies are described: mouse monoclonal antibody to methylarginine, clone 7E6 (IgG1) (Abcam, Cambridge, UK) (Data sheet: www.abcam.com/public/ab_detail.cfin?intAbID=412, which reacts with mono- and asymmetric dimethylated arginine residues; and mouse monoclonal antibody to methylarginine, clone 21C7 (IgM) (Abcam) (Data sheet: www.abcam.com/public/ab_detail.cfm?intAbID=413), which reacts with asymmetric dimethylated arginine residues. Detailed protocols for in vitro and in vivo analysis of arginine methylation are provided. See Mowen et al., Cell 104: 731-741, 2001.

Even if there is no reported antibodies at present for certain specific modifications, it is well within the capability of a skilled artisan to raise antibodies against that specific type of modified residues. There is no compelling reason to believe that such antibodies cannot be obtained, especially in view of the prior success in raising antibodies against reletively small groups such as phosphorylated amio acids. The anti-post-translational modification antibody should be checked against the same antigen that is un-modified to verify that the reactivity is depending upon the presence of the post-translational modification.

G. Immunohistochemistry (IHC)

Immunohistochemical analysis of tumor tissues/biopsy has traditionally played an important role in diagnosis, monitoring, and prognosis analysis of cancer. IHC is typically performed on disease tissue sections using antibodies (monoclonal or polyclonal) to specific disease markers. However, two major problems have hampered this useful procedure, such that it is frequently difficult to get reproducible, quantitative data. One problem is associated with the poor quality of antibodies used in the assay. Many antibodies lack specificity to a target biomarker, and tend to cross-react with other proteins not associated with disease status, resulting in high background. The other complication is that antibody may have difficulties accessing unknown epitopes after tissue/cell fixation.

For example, Press et al. (Cancer Res. 54(10): 2771-7, 1994) compared immunohistochemical staining results obtained with 7 polyclonal and 21 monoclonal antibodies in sections from paraffin-embedded blocks of breast cancer samples. It was found that the ability of these antibodies to detect the HER2/neu antigen overexpression was extremely variable, providing an important explanation for the variable overexpression rate reported in the literature.

The other problem is associated with sample processing before IHC. Generally, the efficiency of antigen retrieval is unpredictable in the concurrent protocol. It is also reported that heating coupled with enzyme digestion tends to give better results. But since epitopes for antibodies are not known, heating/digestion may cause different degree of problems for antibody recognition.

Therefore, PET-derived antibodies represent a unique solution as standardized reagents for IHC. In certain preferred embodiments, PETs present on the surface of the target protein will be chosen for easy accessibility by the PET-specific antibodies. The chemistry of cell fixation may also be taken into account to select optimum amino acid sequences of PETs. For example, if certain residues are known to form cross-links after fixation, these residues will be selected against in PET selection. Similarly, epitopes that overlap with enzyme recognition sites will not be chosen. These measures will help to achieve consistent, reproducible results and high rate of success in IHC experiments.

VII. Use of Multiple PETs in Highly Accurate Functional Measurement of Proteins

In certain embodiments of the invention, it may be advantageous to produce two or more PETs for each protein of interest. For example, trypsin digestion (or any other protease treatment or chemical fragmentation methods described above) may be incomplete or biased for/against certain fragments. Similarly, recovery of fragmented polypeptides by PET-specific capture agents may occasionally be incomplete and/or biased. Therefore, there may be certain risks associated with using one specific PET-specific capture agent for measurement of a target polypeptide.

To overcome this potential problem, or at least to compensate for the above-described incomplete digestion/recovery problems, two or more PETs specific to the polypeptide of interest may be generated, and used on the same array of the instant invention, or used in the same set of competition assays to independently detect different PETs of the same polypeptide. The average measurement results obtained by using such redundant PET-specific capture agents should be much more accurate and reliable when compared to results obtained using single PET-specific capture agents.

On the other hand, certain proteins may have different forms within the same biological sample. For example, proteins may be post-translationally modified on one or more specific positions. There are more than 100 different kinds of post-translational modifications, with the most common ones being acetylation, amidation, deamidation, prenylation, formylation, glycosylation, hydroxylation, methylation, myristoylation, phosphorylation, ubiquitination, ribosylation and sulphation. For a specific type of modification, such as phosphorylation, a PET peptide phosphorylated at a site may not be recognized by a capture agent raised against the same but unphosphorylated PET pepetide. Therefore, by comparing the result of a first capture agent specific for unmodified PET peptide of a target protein (which represents unmodified target protein), with the result of a second capture agent specific for another PET within the same target protein (which does not contain any phosphorylation sites and thus representing the total amount of the taget protein), one can determine the percentage of phosphorylated target protein within said sample.

The same principle applies to all target proteins with different forms, including unprocessed/pre-form and processed/mature form in certain growth factors, cytokines, and proteases; alternative splicing forms; and all types of post-translational modifications.

In certain embodiments, capture agents specific for different PETs of the same target protein need not be of the same category (e.g., one could be an antibody specific for PET1, the other could be non-antibody binding protein for PET2, etc.)

In other embodiments, the presence or absence of one or more PETs is indicative of certain functional states of the target protein. For example, some PETs may be only present in unprocessed forms of certain proteins (such as peptide hormones, growth factors, cytokines, etc.), but not present in the corresponding mature/processed forms of the same proteins. This usually arises from the situation where the processing site resides within the PETs. On the other hand, other PETs might be common to both precessed and unprocessed forms (e.g., do not contain any processing sites). If both types of PETs are used in the same array, or in the same competition assay, the abundance and ratio of processed/unprocessed target protein can be assessed.

In other embodiments, due to the vastly improved overall accuracy of the measurement using multiple PET-specific capture agents, the invention is applicable to the detection of certain previously unsuitable biomarkers because they have low detectable level (such as 1-5 pM) which is easily obscured by background signals. For example, as described above, Punglia et al. (N. Engl. J. Med. 349(4): 335-42, July, 2003) indicated that, in the standard PSA-based screening for prostate cancer, if the threshold PSA value for undergoing biopsy were set at 4.1 ng per milliliter, 82 percent of cancers in younger men and 65 percent of cancers in older men would be missed. Thus a lower threshold level of PSA for recommending prostate biopsy, particularly in younger men, may improve the clinical value of the PSA test. However, at lower detection limits, background can become a significant issue. The sensitivity/selectivity of the multiple PET-specific capture agent assay can be used to relaibly and accurately detect low levels of PSA.

Similarly, due to the increased accuracy of measurements, small changes in concentration are more easily and reliably detected. Thus, the same method can also be used for other proteins previously unrecognized as disease biomarkers, by monitoring very small changes of protein levels very accurately. “Small changes” refers to a change in concentration of no more than about 50%, 40%, 30%, 20%, 15%, 10%, 5%, 1% or less when comparing a disease sample with a normal/control sample.

Accuracy of a measurement is usually defined by the degree of variation among individual measurements when compared to the true value, which can be reasonably accurately represented by the mean value of multiple independent measurements. The more accurate a method is, the closer a random measurement will be as compared to the mean value. A x % accuracy measurement means that x % of the measurements will be within one standardized deviation of the mean value. The method of the invention is usually at least about 70% accurate, preferably 80%, 90% or more accurate.

Detection of the presence and amount of the captured PET-containing polypeptide fragments can be effectuated using any of the methods described above that are generally applicable for detecting/quantitating the binding event.

To reiterate, for example, for each primary capture agent on an array, a specific, detectable secondary capture agent might be generated to bind the PET-containing peptide to be captured by the primary capture agent. The secondary capture agent may be specific for a second PET sequence on the to be captured polypeptide analyte, or may be specific for a post-translational modification (such as phosphorylation) present on the to-be-captured polypeptide analyte. To facilitate detection/quantitation, the secondary capture agent may be labeled by a detectable moiety selected from: an enzyme, a fluorescent label, a stainable dye, a chemilumninescent compound, a colloidal particle, a radioactive isotope, a near-infrared dye, a DNA dendrimer, a water-soluble quantum dot, a latex bead, a selenium particle, or a europium nanoparticle.

Alternatively, the captured PET-containing polypeptide analytes may be detected directly using mass spectrometry, colorimetric resonant reflection using a SWS or SRVD biosensor, surface plasmon resonance (SPR), interferometry, gravimetry, ellipsometry, an evanascent wave device, resonance light scattering, reflectometry, a fluorescent polymer superquenching-based bioassay, or arrays of nanosensors comprising nanowires or nanotubes.

Another aspect of the invention provides arrays comprising redundant capture agents specific for one or more target proteins within a sample. Such arrays are useful to carry out the methods described above (e.g. high accuracy functional measurement of the target proteins). In one embodiment, several different capture agents are arrayed to detect different PET-containing peptide fragment derived from the same target protein. In other embodiments, the array may be used to detect several different target proteins, at least some (but may be not all) of which may be detected more than once by using capture agents specific for different PETs of those target proteins.

Another aspect of the invention provides a composition comprising a plurality of capture agents, wherein each of said capture agents recognizes and interacts with one PET of a target protein. The composition can be used in an array format in an array device as described above.

VIII. Other Aspects of the Invention

In another aspect, the invention provides compositions comprising a plurality of isolated unique recognition sequences, wherein the unique recognition sequences are derived from at least 50%, 55%, 60%, 65%, 70%, 75%, 80%, 85%, 90% 95% or 100% of an organism's proteome. In one embodiment, each of the unique recognition sequences is derived from a different protein.

The present invention further provides methods for identifying and/or detecting a specific organism based on the organism's Proteome Epitope Tag. The methods include contacting a sample containing an organism of interest (e.g., a sample that has been fragmented using the methods described herein to generate a collection of peptides) with a collection of unique recognition sequences that characterize, and/or that are unique to, the proteome of the organism. In one embodiment, the collection of unique recognition sequences that comprise the Proteome Epitope Tag are immobilized on an array. These methods can be used to, for example, distinguish a specific bacterium or virus from a pool of other bacteria or viruses.

The unique recognition sequences of the present invention may also be used in a protein detection assay in which the unique recognition sequences are coupled to a plurality of capture agents that are attached to a support. The support is contacted with a sample of interest and, in the situation where the sample contains a protein that is recognized by one of the capture agents, the unique recognition sequence will be displaced from being bound to the capture agent. The unique recognition sequences may be labeled, e.g., fluorescently labeled, such that loss of signal from the support would indicate that the unique recognition sequence was displaced and that the sample contains a protein is recognized by one or more of the capture agents.

The PETs of the present invention may also be used in therapeutic applications, e.g., to prevent or treat a disease in a subject. Specifically, the PETs may be used as vaccines to elicit a desired immune response in a subject, such as an immune response against a tumor cell, an infectious agent or a parasitic agent. In this embodiment of the invention, a PET is selected that is unique to or is over-represented in, for example, a tissue of interest, an infectious agent of interest or a parasitic agent of interest. A PET is administered to a subject using art known techniques, such as those described in, for example, U.S. Pat. No. 5,925,362 and international publication Nos. WO 91/11465 and WO 95/24924, the contents of each of which are incorporated herein by reference. Briefly, the PET may be administered to a subject in a formulation designed to enhance the immune response. Suitable formulations include, but are not limited to, liposomes with or without additional adjuvants and/or cloning DNA encoding the PET into a viral or bacterial vector. The formulations, e.g., liposomal formulations, incorporating the PET may also include immune system adjuvants, including one or more of lipopolysaccharide (LPS), lipid A, muramyl dipeptide (MDP), glucan or certain cytokines, including interleukins, interferons, and colony stimulating factors, such as IL1, IL2, gamma interferon, and GM-CSF.

### EXAMPLES

This invention is further illustrated by the following examples which should not be construed as limiting. The contents of all references, patents and published patent applications cited throughout this application, as well as the Figures are hereby incorporated by reference.

In addition, certain examples as disclosed in the parent applications U.S. Ser. No. 10/773,032 (filed on Feb. 5, 2004), U.S. Ser. No. 10/712,425 (filed on Nov. 13, 2003), and U.S. Ser. No. 10/436,549 (filed on May 12, 2003), including “IDENTIFICATION OF UNIQUE RECOGNITION EQUENCES WITHIN THE HUMAN PROTEOME,” “IDENTIFICATION OF UNIQUE RECOGNITION SEQUENCES (OR PETS) WITHIN ALL BACTERIAL PROTEOMES,” “IDENTIFICATION OF SPECIFIC PETS,” detection and generation of SARS-specific PETS, alone with their associated Figures and sequences, are all incorporated herein by reference.

### Example 1

### Detection and Quantitation in a Complex Mixture of a Single Peptide Sequence with Two Non-Overlapping Pet Sequences Using Sandwich Elisa Assay

A fluorescence sandwich immunoassay for specific capture and quantitation of a targeted peptide in a complex peptide mixture is illustrated herein.

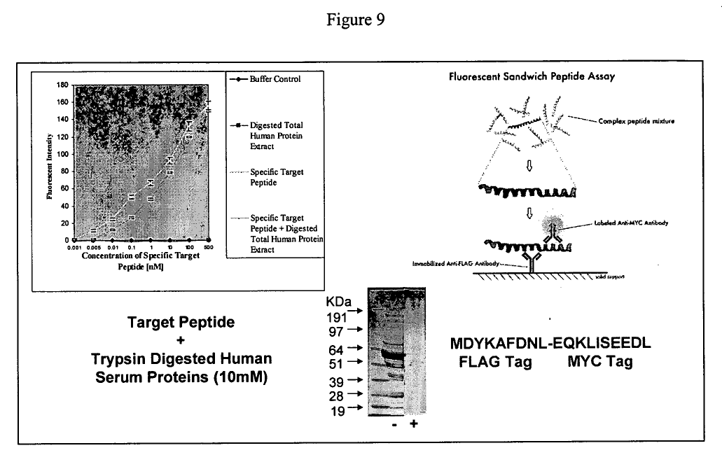

In the example shown here, a peptide consisting of three commonly used affinity epitope sequences (the HA tag, the FLAG tag and the MYC tag) is mixed with a large excess of unrelated peptides from digested human protein samples (FIG. 9). The FLAG epitope in the middle of the target peptide is first captured here by the FLAG antibody, then the labeled antibody (either HA mAb or MYC mAb) is used to detect the second epitope. The final signal is detected by fluorescence readout from the secondary antibody. FIG. 9 shows that picomolar concentrations of HA-FLAG-MYC peptide was detected in the presence of a billion molar excess of digested unrelated proteins. The detection limit of this method is typically about 10 pM or less.

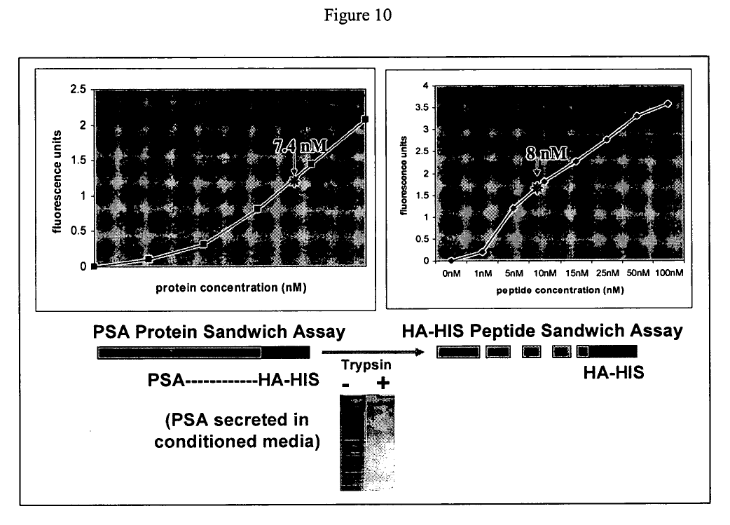

The sandwich assay was used to detect a tagged-human PSA protein, both as full length protein secreted in conditioned media of cell cultures, and as tryptic peptides generated by digesting the same conditioned media. The result of this analysis is shown in FIG. 10. The PSA protein sandwich assay (left side of the figure) indicated that the PSA protein concentration is about 7.4 nM in conditioned media. SDS-PAGE analysis indicated that the tryptic digestion of all proteins in the sample was complete, manifested by the absence of any visible bands on the gel after digestion since most tryptic fragments are expected to be less than 1 kDa. The right side of the figure indicated that nearly the same concentration (8 nM) of the last fragment—the tag-containing portion of the recombinant PSA protein was present in the digested sample. The higher concentration could be attributed to the elimination of interfering substances in the sample, such as other proteins that bind the full-length PSA protein and mask its interaction with the antibody. Although this type of interference is not so severe in this example since the relatively simple conditioned media was used, it is expected to be much more prevalent in real biological samples, where large interference is expected from unknown proteins in a non-digested and complicated bodily fluid such as serum.

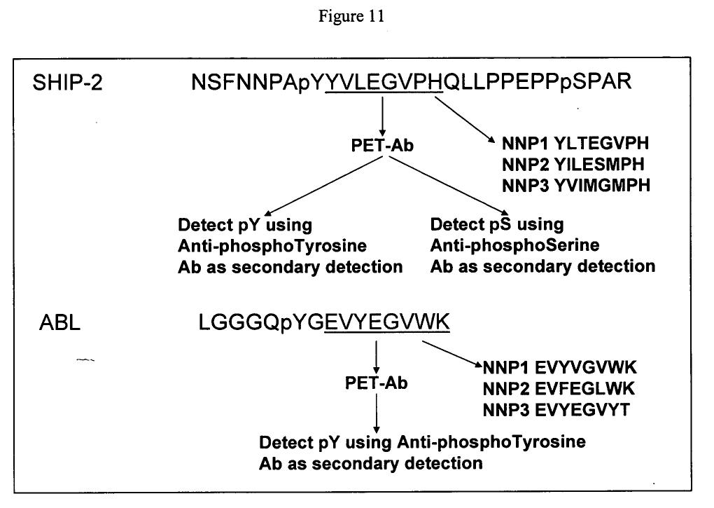

The same sandwich assay may be used for detecting modified amino acids, such as phosphorylated proteins using anti-tyrosine, anti-serine, or anti-threonine antibodies. For example, FIG. 11 shows that the phopshoprotein SHIP-2 contains a 28-amino acid tryptic fragment, which is phosphorylated on one tyrosine residue N-terminal to an 8-mer PET (YVLEGVPH) and on one serine residue C-terminal to the PET. Thus in the sandwich assay, the trypsin digested SHIP-2 protein can first be pulled-down using the PET-specific antibody, and the presence of phosphorylated tyrosine or serine may be detected/quantitated using the phospho-specific antibodies, such as those described elsewhere in the instant specification. Three of the nearest neighbors of the selected PET are also shown in the figure.

Similarly, the phosphoprotein ABL also contains an 8-mer PET on its tryptic fragment containing the phosphorylation site. The phosphorylated peptide is readily detectable by a phospho-tyrosine-specific antibody.

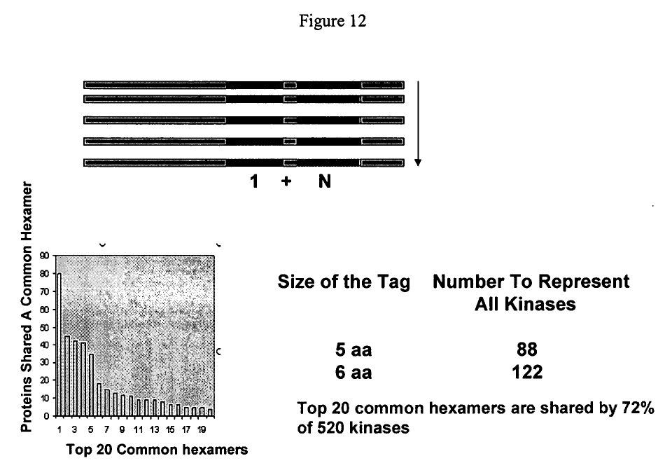

In fact, as a general approach, the sandwich assay may be used to detect N proteins with N+1 PET-specific antibodies: one PET is common to all N peptides to be detected, while each specific peptide also contains a unique PET. All N peptides can be pulled-down by a capture agent specific to the common PET, and the presence and quantity of each specific peptide can be individually assessed using antibodies specific to the unique PETs (see FIG. 12).

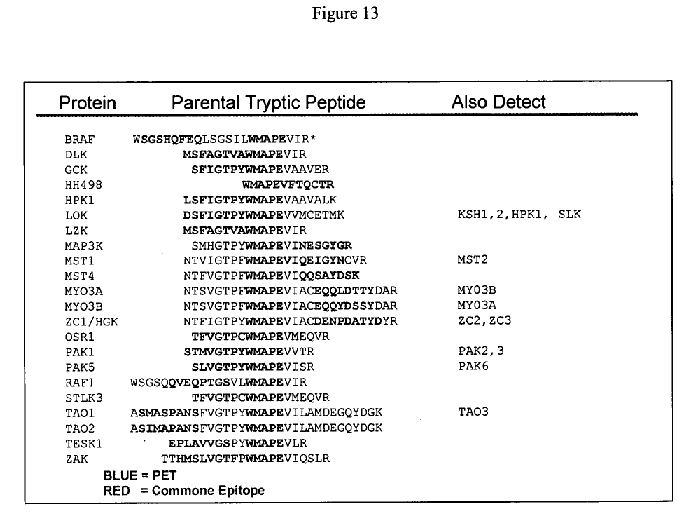

To illustrate, most kinases are somehow related by sharing similar catalytic structures and/or catalytic mechanisms. Thus, it is interesting that only 88 5-mer PETs are needed to represent all known 518 human kinases, and 122 6-mer PETs are needed for the same purpose. FIG. 12 also shows that the top 20 most common 6-mer PETs cover more than 70% of all known kinases. Since closely related kinases tend to share common features, the subject sandwich assay is suitable for simultaneous detection of family of kinases. FIG. 13 provides such an example, wherein one 5-mer PET is shared among tryptic fragments of 22 related kinases, each of which also has unique 7-mer or 8-mer PETs.

The same approach may be used for other protein families, including GPCRs, proteases, phosphotases, receptors, or specific enzymes. The Human Plasma Membrane Receptome (HPMR) is disclosed at Stanford University's receptome website.

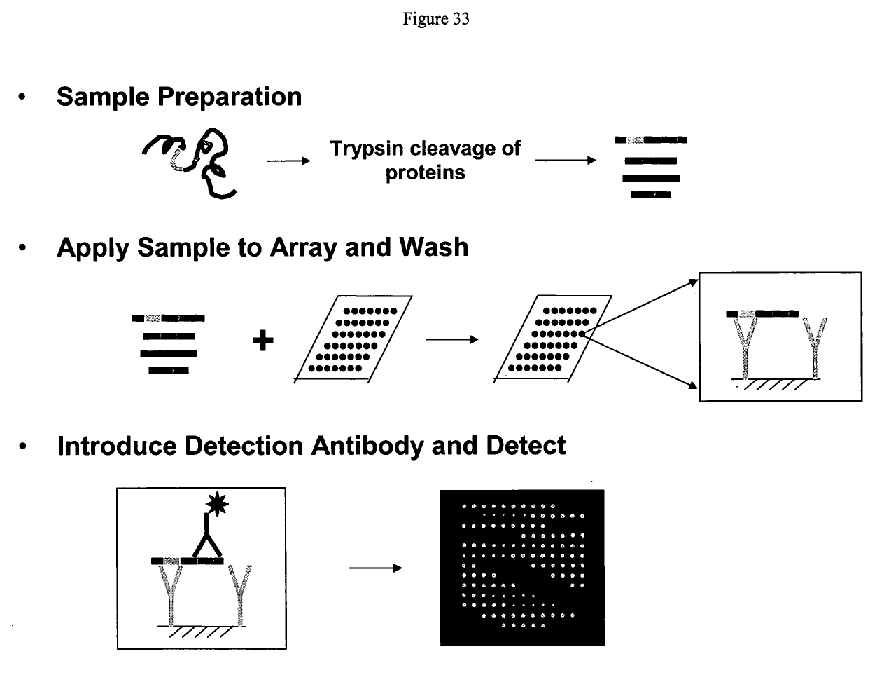

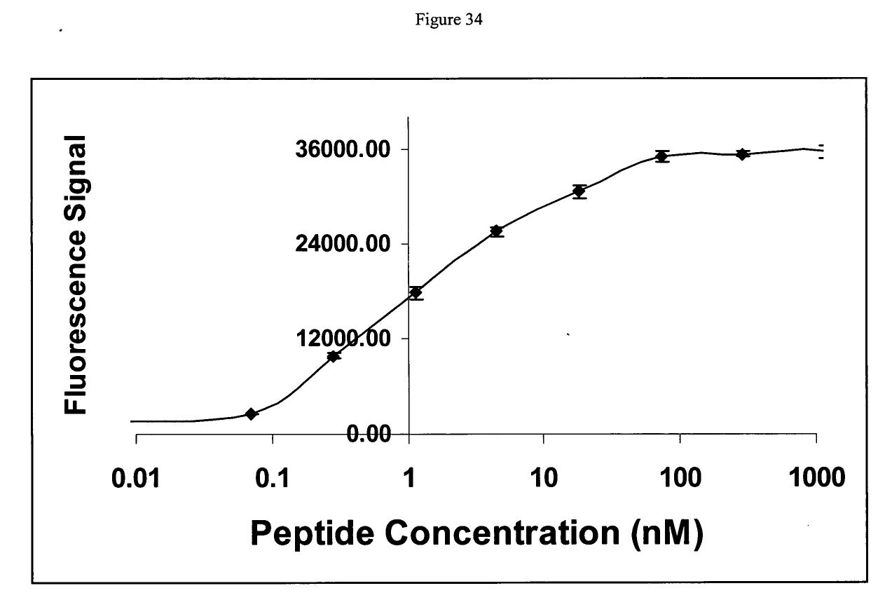

FIG. 33 is a schematic drawing illiustrating the general approach of antibody sandwich assay. FIG. 34 shows an exemplary result of sandwich assay for PSA detection. Two PET antibodies were used to detect a PSA tryptic fragment.

Specifically, capture antibody was printed on commercially-available Poly-L Lysine slides (CEL Associates, Inc.) using a non-contact PiezzoArray printer (Perkin Elmer). A single 350 pL volume of antibody, at a concentration of 0.5 mg/ml was printed. The slide was blocked with a 6% BSA solution for 2 hours, and then excess BSA was removed. The standard curve was constructed using a synthetic peptide that represents a PSA tryptic fragment at concentrations varying from 0.01 to 1000 nM. The standard curve was prepared in a PBS buffer containing 6% BSA. Following a 1 hour incubation, the slide was washed five times in a PBS buffer containing 0.05% tween. Detection antibody labeled with Alex Fluor 555 (Molecular Probes) at a concentration of 10 nM was introduced. Following a 1 hour incubation, the slide was washed again and then scanned in a fluorescent scanner (Scan Array HT, Perkin Elmer). The images were analyzed using the QuantArray software and the data was reduced on an Excel Spreadsheet.

### Example 2

### Peptide Competition Assay

In certain embodiments of the invention, a peptide competition assay may be used to determine the binding specificity of a capture agent towards its target PET, as compared to several nearest neighbor sequences of the PET.

For a typical peptide competition assay, the following illustrative protocol may be used: 1 μg/100 μl/well of each target peptide is coated in Maxisorb Plates with coating buffer (carbonate buffer, pH 9.6) overnight at 4° C., or 1 hour at room temperature. The plates are washed with 300 μl of PBST (1×PBS/0.05% tween 20) for 4 times. Then 300 μl of blocking buffer (2% BSA/PBST) is added and the plates are incubated for 1 hour at room temperature. Following blocking, the plates are washed with 300 μl of PBST for 4 times.

Synthesized competition peptides are dissolved in water to a final concentration of 2 mM solution. Serial dilution of competition peptides (for example, from 100 pM to 100 μM) in digested human serum are prepared. These competition peptides at particular concentrations are then mixed with equal amounts of primary antibodies against the target peptide. These mixtures are then added to plate wells with immobilized target peptides respectively. Binding is allowed to proceed for 2 hours at room temperature. The plates are washed with 300 μl of PBST for 4 times. Then labeled secondary antibody against the primary antibody, such as 100 μl of 5,000× diluted anti-rabbit-IgG-HRP, is added and incubated for 1 more hour at room temperature. The plates are washed with 300 μl of PBST for 6 times. For detection of the HRP label activity, add 100 μl of TMB substrate (for HRP) and incubate for 15 minutes at room temperature. Add 100 μl of stop buffer (2N HCL) and read the plates at OD₄₅₀. A peptide competition curve is plotted using the ABS at OD₄₅₀ versus the competitor peptide concentrations.

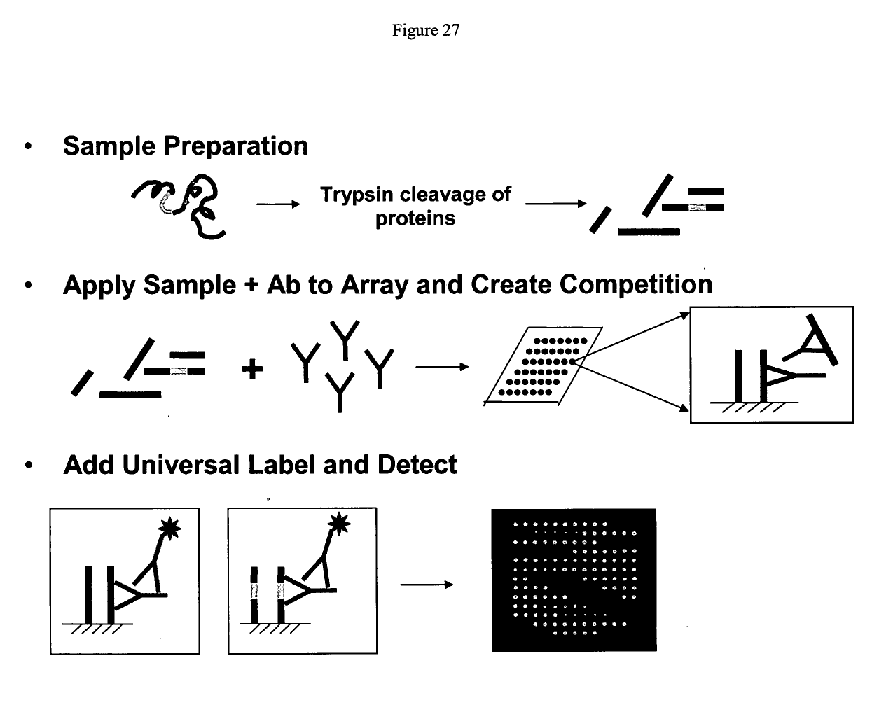

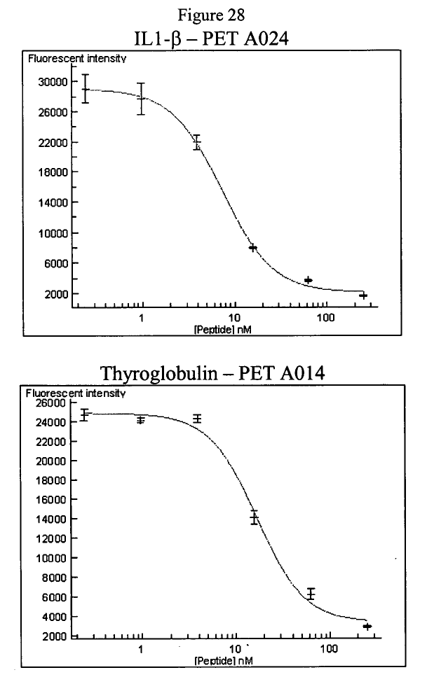

FIG. 27 is a schematic drawing illiustrating the general approach of the peptide array competition assay. FIG. 28 shows exemplary standard competition curves for two of the arrayed PET peptides A024 (IL1-β) and A014 (Thyroglobulin).

Peptide was printed on commercially available Amino Silane slides (ES) using a non-contact PiezzoArray printer (Perkin Elmer). A single 350 pL volume of peptide, at a concentration of 25 μM was printed. The slide was blocked with a 6% BSA solution for 2 hours, and excess BSA was then removed. The standard curve was prepared using a soluble version of the same peptide that was printed on the slide at concentrations varying from 0.01 to 1000 nM, in combination with a constant amount of anti-PET antibody. At low soluble peptide concentration, the antibody binded exclusively to the peptide on the array and maximum assay signal was generated. As soluble peptide concentrations increased, competition for antibody binding increased and less antibody binded to the peptide on the array. The standard curve was prepared in a PBS buffer containing 6% BSA. Following a 1 hour incubation, the slide was washed five times in a PBS buffer containing 0.05% tween. A goat-anti-rabbit detection antibody at a concentration of 2 nM, labeled with Alex Fluor 555 (Molecular Probes) was introduced. Following a 1 hour incubation, the slide was washed again and then scanned in a fluorescent scanner (Scan Array HT, Perkin Elmer). The images were analyzed using the QuantArray software and the data was reduced on an Excel Spreadsheet.

### Example 3

### Pet-Specific Antibodies are Highly Specific and have High Affinity for their Pet Antigens

There are numerous PET-specific antibodies that were shown to be highly specific and have high affinity for their respective antigens. The following table lists a few exemplary antibodies showing high affinity (low nanomolar to high picomolar range) for their respective antigens.

| | Length | Affinity | |
|---|---|---|---|
| Peptide Sequence | (aa) | (K_{D} in nM) | Reference |
| GATPEDLNQKLAGN | 14 | 1.4 | Cell 91: 799, 1997 |
| CRGTGSYNRSSFESSSG | 17 | 2.8 | JIM 249: 253, 2001 |
| NYRAYATEPHAKKKS | 15 | 0.5 | EJB 267: 1819, |
| | | | 2000 |
| RYDIEAKVTK | 10 | 3.5 | JI 169: 6992, 2002 |
| DRVYIHPF | 8 | 0.5 | JIM 254: 147, 2001 |
| PQSDPSVEPPLS | 12 | 16 (a scFv) | NG 21: 163, 2003 |
| YDVPDYAS (HA tag) | 8 | 2 | engeneOS |
| MDYKAFDN (FLAG tag) | 8 | 2.3 | engeneOS |
| HHHHH (HIS tag) | 5 | 25 | Novagen |

Further more, the table below shows three additional PET-specific antibodies with similar nanomolar-range affinity for the respective antigens:

| PET Sequence | Ab name | Affinity (K_{D} in nM) | Parental Protein |
|---|---|---|---|
| EPAELTDA | P1 | 5 | PSA |
| YEVQGEVF | C1 | 31 | CRP |
| GYSIFSYA | C2 | 200 | CRP |

These PETs are selected based on the criteria set forth in the instant specification, including nearest neighbor analysis. Listed below are several nearest neighbors of two of the PETs above.

| PET | LSEPAELTDAVK | AA Differences |
|---|---|---|
| NNP1 | DEPVELTSAPTGHTFS | 2 |
| NNP2 | AGEAAELQDAEVESSAK | 2 |
| NNP3 | LQEPAELVESDGVPK | 3 |
| NNP4 | AQPAELVDSSGW | 3 |
| NNP5 | GLDPTQLTDALTQR | 3 |
| PET | YEVQGEVFTK | AA Differences |
| NNP1 | HVEVNGEVFQK | 2 |
| NNP2 | SYEVLGEEFDR | 2 |
| NNP3 | QYAVSGEIFVVDR | 3 |
| NNP4 | VYEEQGEIILK | 3 |
| NNP5 | LYEVRGETYLK | 3 |

PET-specific antibodies are not only high affinity antibodies, but also highly specific antibodies showing little, if any cross-reactivity with other closely related peptide sequences.

For example, FIG. 17 shows peptide competition results using the peptide competition assay described in Example 2. The left panel shows that antibody P1, which is specific for the PSA-derived 8-mer PET sequence EPAELTDA, can be effectively competed away by the antigen PET (EPAELTDA), with a half-maximum effective peptide concentration of around 40 nM. However, two of its nearest-neighbor 8-mer PETs found in the human proteome with only two- or three-amino-acid differences, EPVELTSA and DPTQLTDA, are completely ineffective even at 1000 μM (25,000-fold higher concentration). Similarly, the right panel shows that antibody C1, which is specific for the CRP-derived 8-mer PET sequence YEVQGEVF, can be effectively competed away by the antigen PET sequence YEVQGEVF, with a half-maximum effective peptide concentration of around 1 μM. However, two of its nearest-neighbor 8-mer PETs found in the human proteome with only two-amino-acid differences, VEVNGEVF and YEVLGEEF, are completely ineffective even at 1000 μM (at least 1,000-fold higher concentration).

### Example 4

### Antibody Cross-Reactivity: Kallikrein Ab's

The kallikreins are a subfamily of the serine protease enzyme family (Bhoola et al., Pharmacol Rev 44: 1-80, 1992; Clements J. The molecular biology of the kallikreins and their roles in inflammation. Farmer S. eds. The kinin system 1997: 71-97 Academic Press New York). The human kallikrein gene family was, until recently, thought to include only three members: KLK1, which encodes for pancreatic/renal kallilrein (hK1); KLK2, which encodes for human glandular kallikrein 2 (hK2); and KLK3, which encodes for prostate-specific antigen (PSA; hK3) (Riegman et al., Genomics 14: 6-11, 1992). The best known of the three classic human kallikreins is PSA, an important biomarker for prostate cancer diagnosis and monitoring. Recently, new serine proteases with high degrees of homology to the three classic kallikreins were cloned. These newly identified serine proteases have now been included in the expanded human kallikrein gene family. The entire human kallikrein gene locus on chromosome 19q13.4 now includes 15 genes, designated KLK1-KLK15; their respective proteins are known as hK1-hK15 (Diamandis et al., Clin Chem 46: 1855-1858, 2000).

KLK13, previously known as KLK-L4, is one of the newly identified kallikrein genes. The protein has 47% and 45% sequence identity with PSA and hK2, respectively (Yousef et al., J Biol Chem 275: 11891-11898, 2000). At the mRNA level, KLK13 expression is highest in the mammary gland, prostate, testis, and salivary glands (Yousef, supra). Although the function of KLK13 is still unknown, KLK13, like all other members of the human kallikrein family, is predicted to encode a secreted serine protease that is likely present in biological fluids. Given the prominent role of PSA as a cancer biomarker and the recent demonstration that other members of this gene family are also potential cancer biomarkers (Diamandis et al., Clin Biochem 33: 369-375, 2000; Luo et al., Clin Chem 47: 237-246, 2001; Diamandis et al., Clin Biochem 33: 579-583, 2000; Luo et al., Clin Chim Acta 7: 806-811, 2001; Diamandis et al., Cancer Res 62: 293-300, 2002), hK13 may also have utility as a disease biomarker. In order to develop a suitable method for measuring hK13 protein in biological fluids and tissues with high sensitivity and specificity, and to further investigate the diagnostic and other clinical applications of this protein, Kapadia et al. (Clinical Chemistry 49: 77-86, 2003) cloned and expressed the full-length recombinant human KLK13 in a yeast expression system, and raised KLK13-specific monoclonal and polyclonal antibodies. A sandwich-type assay revealed that the KLK13 antibody is quite specific—recombinant hK1, hK2, hK3, hK4, hK5, hK6, hK7, hK8, hK9, hK10, hK11, hK12, hK14, and hK15 proteins did not produce measurable readings, even at concentrations 1000-fold higher than that of hK13.

However, it should be noted that this type of antibody specificity defined by cross-reactivity to other related proteins, without any epitope information, can frequently be misleading, and thus the data presented in Kapadia et al. should be interpreted with caution. For one thing, unrelated proteins may have higher sequence homology or conformation similarity than family proteins. It may be pure luck that any hK13 antibody does not cross-react with other highly related family members. However, there is no guarantee that the specific epitope recognized by the hK13 antibody does not appear in other proteins, such as an un-identified kallikrein family member, or an alternative splicing form of hK13. Therefore, antibody specificity is better defined by reactivity to peptides most homologous to a selected PET (nearest neighbor peptides). Antibody cross-reactivity is now readily measurable using peptide competitive assays at a wide dynamic range.

On the other hand, in certain situations, detection for the whole protein family or a specific subset of the family are needed. For example, it has already been demonstrated that multiple kallikreins are overexpressed in ovarian carcinoma (reviewed in Yousef and Diamandis, Minerva Endocrinol 27: 157-166, 2002). There is experimental evidence that these kallikreins may form a cascade enzymatic pathway similar to the pathways of coagulation and fibrinolysis. Therefore, one single antibody specific for the subset of ovarian carcinoma-associated kallikreins is of particular interest in clinical setting. Lastly, the concentrations of competitors used is limited in Kapadia's assay.

These problems can be readily tackled with the approach of the instant invention. For example, the table below lists a common PET for hK1-hK11 (except hK6 and 7, which have their common PETs), as well as PETs specific for each hK proteins listed. In addition, both the family-specific PET and the protein-specific PET are within the same tryptic fragment.

| | |
|---|---|
| hK1 | HSQPWQVAVYSHGWAHCGGVLVHR |
| hK2 | IVGGWECEQHSQPWQAALYHFSTFQCGGILVHK |
| hK3 | GSQPWQVSLFNGLSFHCAGVLVDR |
| hK4 | NSQPWQVGLFEGTSLR |
| hK5 | HECQPHSQPWQAALFQGQQLLCGGVLVGR |
| hK8 | EDCSPHSQPWQAALVMENELFCSGVLVHR |
| hK9 | VLNTNGTSGFLPGGYTCFPHSQPWQAALLVQGR |
| hK10 | LLEGDECAPHSQPWQVALYER |
| hK11 | PNSQPWQAGLFHLTR |
| hK6 | CVTAGTSCLISGWGSTSSPQLR |
| Hk7 | VMDLPTQEPALGTTCYASGWGSIEPEEFLTPK |

By using these family- and individual-specific PET antibodies (or other suitable capture reagents), the same tryptic digestion can be used for a sandwich-type assay that captures all interested tryptic peptides (using the family-specific PET antibodies), followed by selective detection/quantitation of specific family members (using for example, differentially labeled individual-specific antibodies, preferably in a single experiment.

In addition, the same approach may be used to detect the presence of alternative splicing isoforms of any protein. For example, there are three alternative splicing forms of

hK15-V1
R*LNPQVR*PAVLPTR*CPHPGEACVVSGWGLVSHEPGTAGSPR*SQG
hK15-V2
R*LNPQ--------------------------------------
hK15-V3
R*LNPQGDSGGPLVCGGILQGIVSWGDVPCDNTTK*PGVYTK

Thus, SGWGLVSH is a PET for detecting V1, with the three nearest neighbor peptides being AGWGIVNH, SGWGITNH, and SGWGMVTE. Similarly, WGDVPCDN is a PET for detecting V1, with the three nearest neighbor peptides being WKDVPCED, WNDAPCDS, and WNDAPCDK.

### Example 5

### Detecting Serum Protein Levels

Due to the fundamental problems in measuring an antigen which exists in more than one form and/or present in different complexes, it may be difficult to reach a consensus on the level of total a serum protein (such as TGF-β1 protein) in normal human plasma. The instant invention provides a method that efficiently solves these problems.

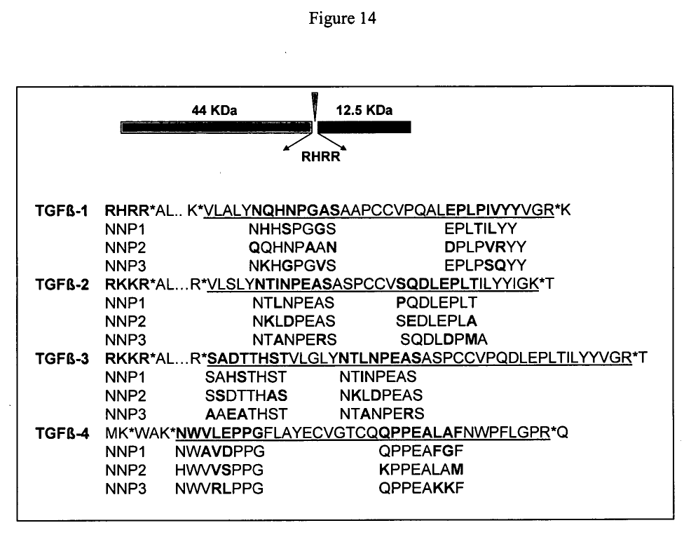

FIG. 14 shows a design for the PET-based assay for standardized serum TGF-beta measurement. The C-terminal monomer for the mature TGF-beta is represented in the top panel as a red bar. The sequences below indicates the PETs specific for each of the 4 TGF-beta isoforms and their respective nearest neighbors. The PET-based assay can be used to specifically detect one of the TGF-beta isoforms, as well as the total amount of all TGF-beta isoforms present in a serum sample.

### Example 6

### Detecting Phospho-Proteins

The PETs of the instant invention may be used to generat proteome-scale affinity reagents and standardized, multiplexed protein assays. PET-based assay transforms protein assays into simplified and standardized peptide assays. In addition, the site-directed nature of the PET approach is especially powerful for protein phosphorylation analysis using high-throughput, highly multiplexed and standardized PET chips. Thus the PET technology is an ideal platform for developing biochips capable of profiling the kinome signaling networks.

The following example demonstrates the feasibility of using the PET-based multiplexed assays for quantitative analysis of proteins. Specifically, we developed a PET-based antibody chip for analyzing the activation of the RAS pathway, by measuring the phosphorylation of ERK1/2 and MEK1/2 proteins. The data include:
Production of high affinity, specific polyclonal antibodies against PET peptides selected from MEK1/2 and ERK1/2 amino acid sequences. The success rate of antibody generation to the PET sequences was close to 100%.
Construction of multiplexed peptide sandwich assays on antibody chips to measure total protein concentration of MEK1/2 and ERK1/2 and phosphorylated ERK1/2; these assays have low pM detection sensitivity (corresponding to ˜1-5 ng/ml protein concentration).
Development of a sample processing and digestion procedure compatible with PET chip assays to generate tryptic peptides from proteins in biological samples
Measuring Ras pathway activation (verified by Western and protein ELISA data) in cultured cells by the increase in phosphorylation of ERK1/2 phosphopeptides using the PET chip.

Thus the complete PET process of going from protein sequences to multiplexed protein assays for measuring cell signaling in complex biological samples has been successfully demonstrated.

1. PET Selection for MEK1/2 and ERK1/2

For this particular example, to select PET sequences, highly unique, highly antigenic, trypsin compatible (do not have internal Arginine and Lysine residues) peptide tags of 8 amino acids in length are calculated by proteome-scale sequence comparison. The uniqueness of a PET peptide is then ranked by identifying the number of PET-homologous peptides in a proteome and the degree of the homology to that particular PET. These homologous peptides, termed nearest neighbor peptides, are defined by pair-wise sequence comparison of a particular PET sequence with the rest of the peptide tags of the same length in a proteome. In most of the cases, PETs that are conserved between human and mouse protein sequences are preferred, so that the same reagents can be used for different species.

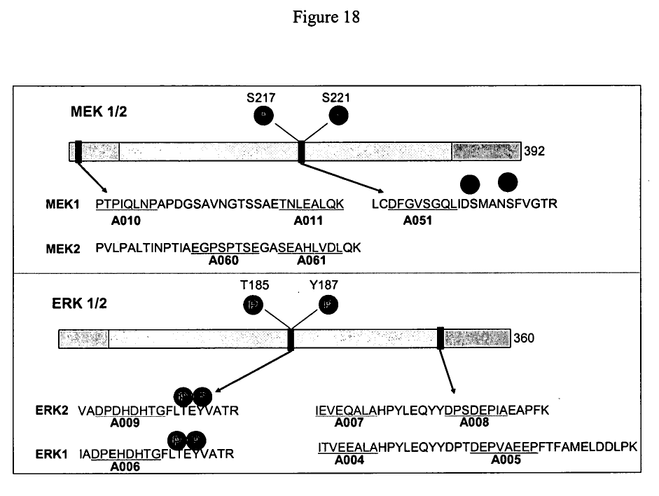

FIG. 18 shows a schematic representation of MEK1/2 and ERK1/2 protein sequences and the relative positions of selected PETs. The tryptic peptide sequences containing the PETs are shown. For each protein, three PETs are selected for three PET antibodies: a sandwich pair that binds one tryptic peptide for total protein concentration measurement, and a third that binds to the targeted phosphotryptic peptide.

Not surprisingly, it was difficult to identify PETs to distinguish highly homologous ERK1 and ERK2 as well as MEK1 and MEK2. For example, the most homologous nearest neighbor peptide in the human proteome for A007 (EQYYDPSD) from ERK2 is an octamer peptide EQYYDPTD from ERK1. Antibodies for these two PETs are likely to be cross-reactive. For ERK total protein measurement, sandwich assays using antibodies A004+A005 or A007+A008 may measure both proteins due to conserved critical amino acid residues in the PET sequences. For MEK total protein measurement, a sandwich assay using A060+A061 may only measure MEK2 while A010+A011 will only measure MEK1. For both ERK and MEK proteins, anti-PET antibodies for A009 (A006) and A051 will measure all phosphorylated ERK1/2 and MEK1/2 proteins, in combination with the appropriate anti-phosphorylation antibody.

PETs selected for ERK1/2 and MEK1/2 are highly unique relative to other proteins in the human proteome. Table A1 shows examples of nearest neighbor peptides for two PETs. The nearest neighbor peptides of selected PETs typically have at least two amino acid residue changes from the PET sequences, increasing the odds of producing specific antibodies against the selected PETs. Many nearest neighbor peptides contain an internal trypsin recognition motif (R and K) and will likely be digested when treated with trypsin.

TABLE A1
Examples of Nearest Neighbor Peptides For PETs

| | | |
|---|---|---|
| PET ID | A010 | A011 |
| PET Sequence | PTPIQLNP | TNLEALQK |
| Nearest Neighbor Peptides | PTPIPLQP | TNLESLEK |
| PTPIQPKP | TNVKALQK | |
| PTPVQTHP | ENLEALQR | |
| PQQIQLNP | SKLEALQK | |
| PTPIEFSP | TNLEALEE | |

*amino acid changes are displayed as underlined letters

For this particular purpose, to accommodate the binding of antibodies to two adjacent octamer PETs on a tryptic peptide for total protein measurement, the tryptic peptide length was selected to be at least 16 amino acids. For tryptic peptides containing phosphorylated amino acids, at least 1 amino acid residue is used to separate the PET and the phosphorylated residue (FIG. 18).

2. High Success Rate of Anti-PET Antibody Production

All 11 PETs shown in FIG. 18 were used to generate antibodies by Abgent Inc. (San Diego, Calif.). For each PET, a total of 6 rabbits were immunized and a total of 66 polyclonal antibodies were generated for the PETs listed. All peptides were synthesized successfully and conjugated to KLH for immunization.

For the 9 tested antibodies, all 54 immunized rabbits showed antigenic responses with the majority of rabbits (49 out 54 or ˜91%) exhibiting high titers when tested by ELISA using immobilized immunogen peptides on 96-well plates. Antibodies from individual rabbits were affinity purified separately using the PET-containing tryptic peptides coupled to a standard chromatography support. On average, we received ˜10 milligrams of affinity purified antibody from each rabbit. All submitted PET sequences gave rise to antibodies, and thus the overall success rate for antibody generation was 100%. The use of multiple rabbits is advantageous since it ensures that at least 1 rabbit will produce antibody for a particular PET.

3. Characterization of Anti-PET Antibodies

a) Anti-PET antibodies have high affinity Since antibody affinity is an important factor for determining immunoassay sensitivity, the affinity of anti-PET antibodies was measured using a surface plasma resonance (SPR) biosensor (Biacore 3000®). Biotinylated tryptic peptides were immobilized on a streptavidin (SA) modified sensorchip and antibody solutions with varying concentrations of antibody were used to flow over the chip. Antibody-peptide binding kinetic measurements were obtained by using a global fit algorithm on the resulting binding curves. Most anti-PET antibodies exhibited a dissociation constant (K_{D}) in the low nanomolar (nM) range. Biacore measurement also gave the kinetics data of kₒₙ and k_{off} rates at which a protein associates or dissociates from its target. Anti-PET antibodies exhibit kₒₙ in the range of 10³-10⁷ M⁻¹s⁻¹ and k_{off} in the range of 10⁻²-10⁻⁵ s⁻¹ measured by Biacore 3000®, indicating that these antibodies will behave similar to high-affinity anti-protein antibodies in immunoassays.

TABLE A2
Affinity of Anti-PET Antibodies by Biacore

| Protein | PET ID | PET-containing Peptide Sequence | K_{D} (nM) |
|---|---|---|---|
| ERK1 | A004 | ITVEEALAHPYLEQ | 6.4 |
| | A005 | DEPVAEEPFTFAMEL | 5.6 |
| pERK1 | A006 | IADPEHDHTG | 25 |
| ERK2 | A007 | IEVEQALAHPYLEQ | 5.9 |
| | A008 | DPSDEPIAEAPFK | 4.1 |
| pERK2 | A009 | VADPDHDHTG | 13.1 |
| MEK1 | A010 | PTPIQLNPAPDG | 26.0 |
| | A011 | GTSSAETNLEALQK | 12.5 |
| pMEK1 | A051 | CDFGVSGQL | 110 |

The affinity of individual antibodies from 6 rabbits for PETs was measured to determine whether antibodies from individual rabbits against the same PET show similar affinity. For most of the PETs, six different antibody populations showed a 4-13 times affinity difference. Table A3 shows the affinity data for different rabbits for selected PETs. This data allows us to select antibodies of highest affinity for subsequent assay construction.

TABLE A3
Affinity Distribution For 6 Rabbits (nM)

| | | | | | Rabbit | Rabbit |
|---|---|---|---|---|---|---|
| PETs | Rabbit 1 | Rabbit 2 | Rabbit 3 | Rabbit 4 | 5 | 6 |
| A007 | 3.14 | 8.09 | 9.41 | 4.10 | 9.21 | 1.72 |
| A008 | 2.55 | 5.33 | 6.44 | 1.77 | 2.57 | 5.97 |
| A009 | 1.94 | 24.9 | 2.30 | 17.00 | 7.20 | 5.97 |
| A010 | 35.80 | 17.00 | 29.00 | 9.00 | 38.00 | 27.00 |
| A011 | 18.00 | 2.00 | 17.00 | 9.00 | 3.00 | 26.00 |

We measured binding of antibodies to closely related PETs for ERK1 and ERK2. As predicted above, antibodies for A004, A005 and A006 bind to tryptic peptides containing A007, A008 and A009, respectively. Based on this data, the following sandwich pairs were selected for assay development: A007+A008 for ERK1/2 total protein and A009 for phosphorylated ERK1/2; A010+A011 for MEK1/2 total protein and A051 for phosphorylated MEK1/2.

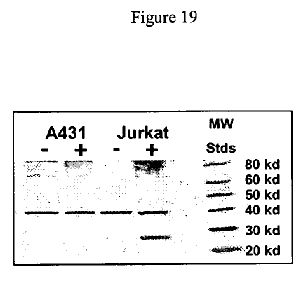

b) Anti-PET antibodies are specific: Specificity of anti-PET antibodies was assessed on Western blots using the anti-PET antibodies as probes on total protein extracts from Jurkat (normal and stimulated with PMA) and A431 cells (normal and stimulated with epidermal growth factor [EGF]). Most of the anti-PET antibodies specifically recognized the target proteins of correct sizes and FIG. 19 shows a representative Western blot for an anti-A008 antibody. The identity of the ˜28 kDa band seen on the stimulated Jurkat cells identified by anti-A008 antibody is not known.

Anti-PET antibody specificity was further assessed by measuring antibody cross-reactivity toward nearest neighbor peptides. Peptide arrays with PET-containing tryptic peptides and predicted nearest neighbor peptides were constructed (Pepscan, The Netherlands) and probed with fluorescently labeled anti-PET antibodies. FIG. 20 shows that anti-PET antibodies failed to bind significantly to selected nearest neighbor peptides.

4. PET Sandwich Assay Development on a Chip

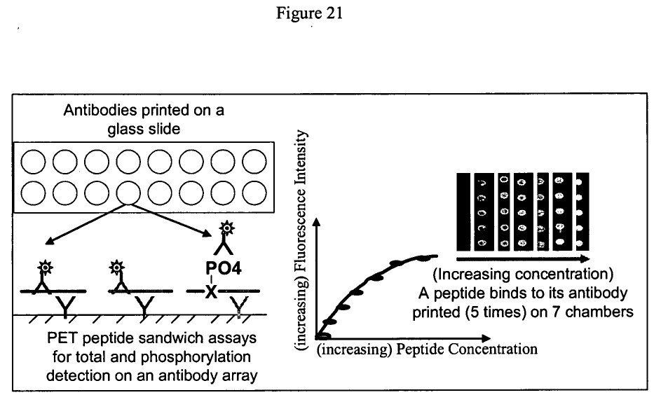

a) Construction of arrays The PET-based sandwich assays were multiplexed on an antibody chip (FIG. 21). Antibody chips were constructed by printing antibodies onto glass slides using a PiezoArray non-contact microarray system (Perkin Elmer, Boston, Mass.). FIG. 21 shows a standard glass microscope slide with a configuration of 16 individual sample chambers each containing an identical array of printed antibodies. This chip configuration is compatible with a 16-pad incubation chamber that sits on top of the slide. Typically antibodies will be printed in 4-6 replicate spots. The piezo tip delivers 350 pL of 0.5 mg/ml antibody solutions to a poly-L-lysine coated glass slide (CEL, Pearland, Tex.) with a spot size of ˜150-200 μm and center to center distance of 300 μm. The printed slides were kept in a vacuumed sealed bag until the time of use.

b) Peptide sandwich assay on arrays: Antibody chip slides with capture antibodies of highest affinity for A007, A010, A009 and A051 were blocked for 2 hours with 6% BSA. A concentration series of standard tryptic peptides (including phosphorylated peptides for A009 and A051) was added to the chambers (100 μL). A sample peptide was mixed with a 1 mg/ml trypsin digested E. coli protein extract to mimic the sample complexity of digested proteins from human cell lines. The reaction was incubated for 1 hour and chambers were washed on an automated microtiter plate washer. Fluorescently labeled sandwich pair antibodies A008, A011 and a commercially available anti-phospho serine antibody for pMEK1/2 or an anti-pERK1/2 antibody were added and the reaction was incubated for 1 hour. The following anti-phosphorylation antibodies were used: monoclonal anti-pERK (p44/p42 MAPK) from Cell Signaling Tech; monoclonal anti-pSer (clone PSR-45) Sigma product # P-3430 and rabbit anti-pSer Biotin RDI product # RDI-PHOSSERR-BT. The chamber containing the anti-phosphorylation antibodies required incubation with a labeled antibody or streptavidin for detection, then all chambers were washed extensively and the slides were visualized on a ScanArray HT microarray scanner (Perkin Elmer, Boston, Mass.). The images were analyzed using the QuantArray software installed on the ScanArray and the data was reduced on an Excel spreadsheet. The concentrations of the capture and secondary detection antibody were varied to yield reasonable detection sensitivity. Table A4 shows the sandwich assay results for ERK1/2 and MEK1/2 total and phospho-tryptic peptides. Overall the sandwich assays detect low pM tryptic peptides in a complex peptide mixture.

TABLE A4
PET Peptide Sandwich Assay Sensitivity

| | Capture | | Sensitivity |
|---|---|---|---|
| Target Peptide | Antibody | Detection Antibody | (pM)* |
| ERK1/2 | | | |
| A007 + A008 | A008 | A007 | ˜33 |
| A009 Phosphopeptide | A009 | Anti-pERK antibody | ˜22 |
| MEK1/2 | | | |
| A010 + A011 | A011 | A010 | ˜59 |
| A051 Phosphopeptide | A051 | Work In Progress | |

*the sandwich assay sensitivity is calculated as the concentration of peptides corresponding to a fluorescence signal intensity at 2 standard deviations from zero concentration of peptides (mean fluorescence intensity at zero peptide concentration + 2 SD of the mean fluorescence intensity at zero peptide concentration)

Based on these results, we proceeded to measure concentrations for total ERK1/2, phosphorylated ERK1/2 and total MEK1/2 using the PET chip in the Ras activation experiments.

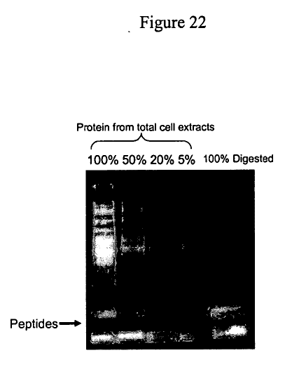

c) Validation of sample processing and digestion protocol Phorbol 12-myristate 13-acetate (PMA) is a protein kinase C activator and PMA treatment of lymphoid cells results in activation of the MEK1->ERK pathway. The human epidermoid cancer cell line A431 over expresses epidermal growth factor receptor (EGFR) and when stimulated with EGF, the Ras pathway is phosphorylated. Jurkat cells or human A431 cells that were 80% confluent were washed with PBS and serum free media was added back to the cells. The cells were incubated overnight and then were taken out of the serum free media, washed and treated with 50 ng/ml PMA or EGF for 10 minutes in serum containing media. The non-stimulated cells were treated in the same way except for the omission of the stimulation step. Cells were then centrifuged and washed with ice-cold PBS. Cells were lysed with standard RIPA (1 ml) buffer (50 mM HEPES, pH 7.4, 150 mM NaCl, 25 mM glycero-phosphate, 25 mM NaF, 5 mM EGTA, 1 mM EDTA, 1% NP-40, 10 g/mL leupeptin, 10 g/mL aprotinin, 1 mM PMSF, 1 mM sodium orthovanadate as phosphatase inhibitors) for 30 min on ice. The lysates were clarified by centrifugation and protein concentrations were determined using the Nanodrop ND-1000 spectrophotometer (NanoDrop, Willmington, Del.). Total protein extracts at a concentration of 1-5 mg/ml was reduced with 5 mM TCEP and 0.05% SDS and heated at 100° C. for 5 minutes. 10 mM of iodoacetamide (100 mM in H₂O) was added into cooled sample and incubated in the dark for 30 minutes. Finally trypsin was added at 1/20^{th} of the starting protein mass and incubated at 37° C. overnight and was then heat inactivated at 100° C. for five minutes. This procedure leaves the sample at or near physiological pH and the reagent addition causes minimal dilution (1.2-1.5 times). FIG. 22 shows a SDS-PAGE gel analysis of total protein (from Jurkat cells) digestion by trypsin. It appears that >95% of the proteins were digested by trypsin to produce peptides.

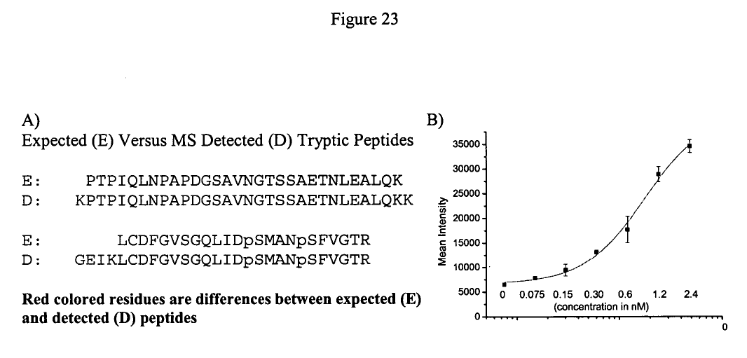

Recombinant phosphorylated MEK1 was digested using the above procedure and analyzed by LC/MS/MS and tryptic peptides corresponding A010+A011 and A051 were detected. These same peptides were then measured by a PET chip assay. The tryptic digest of different amounts of MEK1 was measured by using the chip sandwich assay using anti-PET antibodies A010+A011. Quantitative correlation between fluorescence signal and amount of digested MEK1 protein was determined as shown in FIG. 23B.

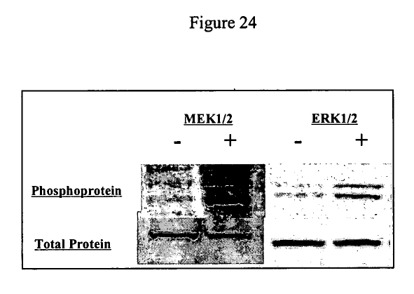

d) Measure the Ras pathway activation by the PET chip: PMA activation of the Ras pathway in Jurkat cells was analyzed by Western blots and commercially available ELISA kits for total and phosphorylated MEK1/2 and ERK1/2. Cell lysates were trypsin digested and tryptic peptides were measured by PET sandwich assays for total (A007+A008), phosphorylated ERK1/2 (A009+anti-pERK antibody) and total MEK1/2 (by A010+A011). As discussed previously, a sandwich assay for phosphorylated MEK1/2 is an ongoing project. Upon stimulation of Jurkat cells with 50 ng/ml PMA, the phosphorylation of MEK and ERK proteins is up-regulated when compared to untreated cells as a negative control as shown in FIG. 24 by Western analysis.

TABLE A5
ELISA and PET Chip Measurement of the Ras Pathway Activation

| | | Total | | | |
|---|---|---|---|---|---|
| | | ERK | pERK | Total MEK | pMEK |
| Un-stimulated | ELISA | 2 nM | NONE | 7 nM | 30 pM |
| Cells | PET | 24 nM | 3 nM | 18.2 nM | No Data |
| | Chip | | | | |
| Stimulated | ELISA | 2 nM | 0.5 pM | 7 nM | 0.15 nM |
| Cells | PET | 21 nM | 12.4 nM | 22 nM | No Data |
| | Chip | | | | |

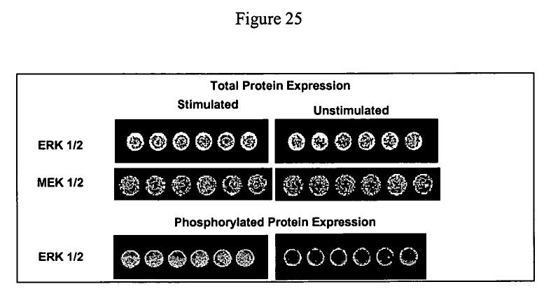

Commercial kits for total protein and phosphorylated protein measurement were used to quantify the activation of ERK1/2 and MEK1/2 (Assay Design, Ann Arbor, Mich.). Recommended protocols from the kits were followed and protein concentrations from the above described cell extracts were measured. Digested cell lysates were measured by PET chip assays. All measured data is shown in Table A5 and fluorescence images of sandwich assays on PET chips for generating data in Table A5 is also shown in FIG. 25. The total ERK1/2 and MEK1/2 concentrations were unchanged in stimulated and unstimulated cell lines measured by PET chips. PET chip assays measured an increase in phosphorylation of ERK1/2, consistent with the activation of the Ras pathway by PMA. The concentrations measured by PET chip assays were generally higher relative to ELISA measurement data. It is consistent with the idea that the digestion of the protein allows a better detection of all possible antibody recognizing epitopes that may be covered in native protein molecules due to protein-protein interaction.

In summary, the Ras activation in Jurkat cells was measured by increased ERK1/2 phophorylation monitored by Western analysis, protein ELISA and PET chip assays. Data presented above demonstrate that PET is a novel technology platform for generating affinity reagents and standardized, multiplexed assays at a proteome-scale. It shows the PET process of going from protein sequences to protein measurement in biological samples using multiplexed PET-based peptide immunoassays. Using the Ras-Raf-MEK-ERK signaling cascade as a model system, we have validated the complete PET process. We have selected PETs using a set of defined rules and these PETs have generated antibodies with a high success rate. One advantage of PET is to pre-define the antibody epitopes in a proteome-scale to “design” antibody specificity. The selection of highly unique peptide sequences relative to all predicted protein sequneces in a proteome increase the odds of generating antibodies of high specificity. Further, it will be straightforward to carry out epitope mapping for anti-peptide antibodies by using peptide arrays consisting of a series of peptide sequences: each peptide sequence will have a single amino acid change into alanine at a particular position while the rest of amino acid residues are unchanged. The initial Western blot analysis and antibody binding using nearest neighbor peptides suggest that anti-PET antibodies have good specificity. Systematic epitope mapping for antibodies generated against a defined library of PETs representing all the predicted human proteins will have tremendous value for understanding the biochemsitry of antibody-peptide interactions. This type of research is expected to yeild more useful information with the help of complete genomic sequence information for many important organisms.

Antibody affinity is an important antibody property. Except for a small number of antibody drugs for clinically important protein targets, affinity measurement is not performed. The affinity for anti-peptide antibodies is much easier to measure than that of anti-protein antibodies since peptides can be synthesized in large quantities and high purity. Anti-PET antibodies generated herein demonstrate high affinity binding to peptide targets. Although selection of octamer PET peptides here is somewhat arbitary, most antibodies only need 2-8 amino acids to interact with for strong peptide or protein binding. Peptides of lengths smaller than 8 amino acid residues may still be used, but could run a higher risk of not being long enough for mounting a appropriate immune response. The affinity of anti-PET antibodies may be better if slighly longer (10-12 amino acids in length) PETs are used to give more epitope choices for the immune system to produce antibodies against.

Table A6 shows the concentrations of total and phosphorylated MEK1/2 and ERK1/2 in Jurkat cells (see Assay Design ELISA kit manuals). The PET chip sandwich assays can detect and measure total ERK1/2, pERK1/2 and total MEK1/2 comfortably from 1-10 million cells/ml (10⁷/ml). We clearly detected a pERK1/2 increase in activated Ras pathway by PMA (Table A5 and attached images).

TABLE A6
MEK1/2 & ERK1/2 Concentrations in Jurkat Cells (nM)

| Number | | | | |
|---|---|---|---|---|
| of Cells | Total ERK1/2 | pERK1/2* | Total MEK1/2 | pMEK1/2# |
| 10⁶/ml | 2 | 0.1 | 0.75 | 0.075 |
| 10⁷/ml | 20 | 1 | 7.5 | 0.75 |
| 10⁸/ml | 200 | 10 | 75 | 7.5 |

*in PMA stimulated cells; in unstimulated cells, pERK1/2 is ˜0.02 nM for 10⁶/ml cells
#in PMA stimulated cells; in unstimulated cells, pMEK1/2 is below detection

Higher sensitivity may be achieved by amplifying signals, such as by using an HRP-based enzyme system. In addition, the assays may be further optimized to ensure that most of the antibodies are oriented properly on surface, the washing and blocking process are optimal, and the secondary detection antibodies are optimally labeled. Further, signaling amplification tools such as rolling circle amplification can be used to enhance the assay sensitivity.

The peptide-based sandwich assays for phosphorylation detection have clear advantages over sandwich assays on native proteins. Production of high specificity reagents is more straightforward and predictable. There is no need to generate specific antibodies for phosphopeptides, which is often a difficult task. The use of a small number of secondary detection antibodies for phosphorylated amino acid residues greatly simplifies the assay design and eliminates potential cross-reactivity among secondary antibodies, an important factor contributing to the lack of suitable antibodies for multiplexing assays. In theory, only one antibody that binds to a phosphate group is needed for all the sandwich assays for phosphorylation detection. Other detecting reagents for phosphorylated Y, T, and S such as the Pro-Q Diamond fluorescence dye developed by Molecular Probes can also be employed.

A sample processing protocol has been developed to produce enzymatically (trypsin) digest proteins in biological samples. The selection of multiple PET containing tryptic peptides for each protein of interest offers assurance that at least one reagent pair is available for the target protein. Furthermore, it may be useful to take advantage of mass spectrometry data to generate antibodies for MS detectable tryptic peptides. These antibodies will be useful for developing chip-based assays and also for enriching target peptides for detection by MS.

Data presented herein demonstrate that PET is a valuable approach for generating defined specificity antibodies. Further, the reagents can be multiplexed in a chip-based assay for measuirng proteins. Instead of dealing with native proteins, PET detects and measures defined peptide tags from trypsin digested samples using antibodies against PETs. PET is applicable to any protein predicted by gene sequence and is made possible by the availablity of complete genomic sequence information of many important organisms. Based on the instant invention, it is possible to design and produce a set of antibodies for a library of PETs defined in the human proteome. These antibodies will then be used to develop array-based assay products to profile important protein classes or subsets, including plasma proteins, cell signaling proteins and cell surface receptors, to name but a few. It is also desirable to develop a PET chip capable of measuring large numbers of cell signaling proteins from multiple pathways.

### Example 7

### Pet-Based Antibody Generation

Using similar methods as described in Example 6 above, we have generated antibodies for several proteins using the PET selection approach. Specifically, we characterized and raise antibodies against 44 PETs from plasma proteins (e.g. TGF-β1, Thyroglobulin, Troponin I, PSA, CRP, Interleukin-1b, C3, C4, C9, Factor XII, FactorB, and Fibronectin) and intracellular proteins for cell signaling (e.g. ERK1, ERK2, MEK1). We also characterized and raise antibodies against 20 PETs from the Kallikrein family (KLK 2, 6, 10) and the complement Family (Factor D, MBL, C1qB). Nine Sandwich pairs are also included. The table below summarizes the results.

| | |
|---|---|
| Number of Antibodies Attempted | 44 |
| Total Number of Rabbits Immunized | 264 |
| Number of Rabbits Responding | 243 (92%) |
| Number of Antibodies Produced | 41 (93%) |

The vast majority of peptide immunogens produced high titer antibody responses in the rabbits. While not a surprising result, it does tell us that the algorithms of the instant invention are predictive of antigenicity, especially when combined with the affinity data.

All antibodies were screened for affinity and specificity. Affinity is comparable among different rabbits. The results are summarized below. About 81% od all antibodies have IC₅₀ in the range of no more than 10 nM.

| Affinity Range (IC₅₀ in nM) | Number | Percent (%) |
|---|---|---|
| <1 nM | 5 | 13 |
| 1-10 nM | 26 | 68 |
| 11-50 nM | 6 | 16 |
| >50 nM | 1 | 3 |

| | | | IC50 in 6% |
|---|---|---|---|
| Protein | PET | Sequence | BSA (nM) |
| TGFbeta1 | A001 | Ac-ALYNQHNPGASAAPC | 0.8 |
| | A002 | Ac-PQALEPLPIVYYVGRC | 3 |
| | A003 | CYIWSLDTQYSK-NH2 | 8 |
| ERK1 | A004 | Ac-ITVEEALAHPYLEQC | 25 |
| | A005 | CVAEEPFTFDMELDD-NH₂ | 6 |
| | A006 | Ac-IADPEHDHTGC | 6 |
| ERK2 | A007 | Ac-IEVEQALAHPYLEQC | 4 |
| | A008 | CDPSDEPIAEAPFK-NH₂ | 6 |
| | A009 | Ac-VADPDHDHTGC | 31 |
| MEK1 | A010 | Ac-PTPIQLNPAPDGC | 36 |
| | A011 | CGTSSAETNLEALQK-NH₂ | 23 |
| Thyroglobulin | A012 | Ac-GGQSAESEEEELC | 10 |
| | A013 | Ac-ELAETGLELLC | 8 |
| | A014 | CEIYDTIFAGLD-NH₂ | 5 |
| | A015 | CDFSTPLAHFDLR-NH₂ | 4 |
| | A016 | CDEAGQELEGMR-NH₂ | 4 |
| Troponin I | A017 | Ac-AYATEPHAKC | 10 |
| | A020 | Ac-NIDALSGMEGRC | 4 |
| PSA | A021 | Ac-LSEPAELTDAVKC | 8 |
| | A022 | CDLPTQEPALG-NH² | 8 |
| | A023 | CGSIEPEEFLTPK-NH₂ | 8 |
| IL-1beta | A024 | CNEDDLFFEADGPK-NH₂ | 0.5 |
| | A025 | Ac-SLVMSGPYELKC | 5 |
| | A026 | Ac-PTLQLESVDPKC | 8 |
| | A027 | CLEFESAQFPNWY-NH₂ | 5 |
| | A028 | CENMPVFLGGTK-NH₂ | 0.5 |
| C3 | A031 | CHYLDETEQWEK-NH₂ | 5 |
| C4 | A034 | CQTDQPIYNPGQR-NH₂ | 4 |
| | A035 | CEANEDYEDYEYDE-NH₂ | 4 |
| C9 | A036 | Ac-TPFDNEFYNGLC | 60 |
| | A037 | CTEHYEEQIEAFK-NH₂ | 0.5 |
| | A039 | Ac-PWNVASLIYETKC | 0.4 |
| CRP | A043 | CYEVQGEVFTK-NH₂ | 4 |
| Fibronectin | A044 | CHEGGQSYK-NH₂ | 8 |
| | A045 | CTFYQIGDSWEK-NH₂ | 1 |
| | A046 | CTTQNYDADQK-NH₂ | 20 |
| | A047 | Ac-VDVIPVNLPGEHGQRC | 10 |
| FactorXII | A049 | CVAGWGHQFEGAEEY-NH₂ | 15 |

Antibody specificity was determined by probing a 33-plex peptide array with antibodies one at a time, showing that each test antibody binds only to the peptide sequence used to generate the antibody.

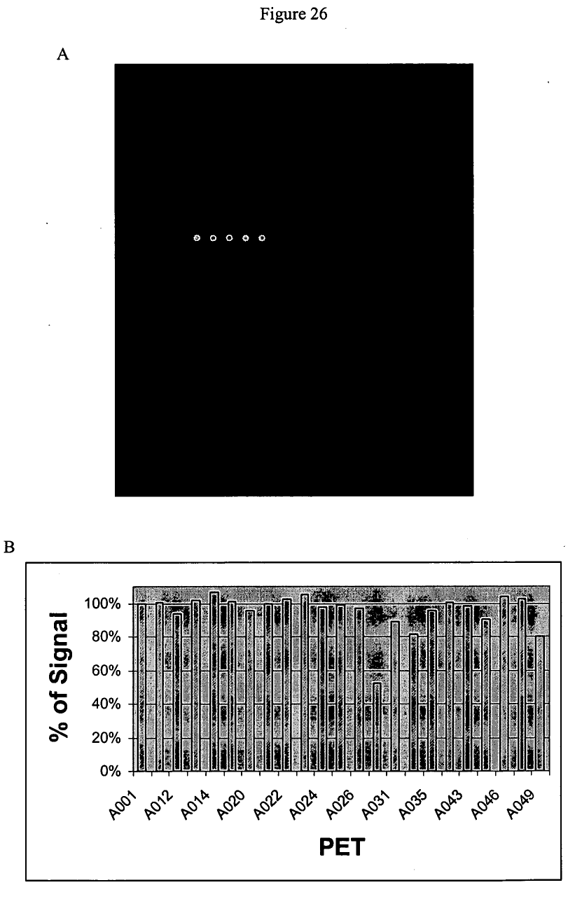

FIG. 26A shows an exemplary result of this series of specificity tests. Only the PET peptide used to raise the tested antibody reacts specifically with the antibody, while the test antibody shows no detectable cross-reactivity towards any of the other 32 peptides on the same array.

Specifically, 33 PET peptides were chosen for the array, include three TGF-beta1, five Thyroglobin, two Troponin I, three PSA, five IL-1beta, two C3, two C4, three C9, one Factor B, one CRP, five Fibronectin, and one Factor VII PET peptides. Also included in the array was the spotting buffer negative control. Each peptide was printed in 5-replicates, with each spot from a 350 pL drop of 25 μM peptide. The resulting array was probed with 100 μl of 5 nM single antibody for 1 hour, followed by 1 hour secondary detection with 100 μl of 2 nM of Goat-anti-Rabbit-Alexa 555 conjugate. The single antibody result shown in FIG. 26A is that of A002-TGF-beta1 antibody.

Antibody specificity is also tested using a competitive assay format, where a penal of PET antibodies were tested against the top four abundant serum proteins (FIG. 26B). This experiment was designed to test the inhibitory effect of high concentrations of peptides, liberated from the most abundant proteins in human serum, on the binding of antibodies to their target PETs.

Unlike many cell lysates, serum is a special biological matrix in that only a few numbers of proteins dominate the composition. Specifically, the proteins listed below account for >95% of total serum protein mass.

| | Concentration (mg/ml) |
|---|---|
| Albumin | 35-45 |
| Gamma globulins | 12-18 |
| IgG | 7-16 |
| IgA | 0.7-4 |
| IgM | 0.4-2 |
| Fibrinogen | 2-6 |
| Alpha-1 antitrypsin | 2-5 |
| Alpha-2 macroglobulin | 2-4 |
| Transferrin | 2-3 |
| Beta-lipoprotein | 4-7 |

To simulate the complexity of a serum digest, we prepared a mixture of the four most abundant serum proteins, namely 50 mg/ml Albumin, 15 mg/ml gamma-globulin, 5 mg/ml alpha-1-antitrypsin, and 5.5 mg/ml transferrin. This mixture, called High Abundance Matrix (H.A.M.) proteins, represents the most common and abundant proteins in serum. If these proteins (or their digested peptides) cross react with the instant PET antibodies, even to a small extent, they will overwhelm the signals expected from the lower abundance proteins we wish to detect.

In this experiment, the HAM mixture was diluted to about 15 mg/ml total protein, reduced with TCEP, alkylated with iodoacetamide and then digested with about 0.75 mg/ml trypsin (i.e. 1/20 by mass) overnight in order to prepare a complex peptide mixture that closely approximates the composition of a serum digest. This mixture of peptides was then used at 3 mg/ml as a matrix in our assays.

To perform the assay, a 33-element peptide array was printed in 16 chambers as described above. One hour primary antibody incubation was followed by detection with 1 hour incubation of 2 nM Goat-anti-Rabbit-Alexa 555 conjugate. The fluorescent intensity of 5 nM antibody (100 μl of 5 nM antibody mixture) diluted in 3 mg/ml digested High Abundance Matrix (HAM) was compared with that of 5 nM antibody diluted in undigested 6% BSA (100 μl of 5 nM antibody mixture). The result was shown as % in FIG. 26B. If there is no inhibition of antibody binding by the peptides released from the digested HAM proteins, the reading should be close to 100%. Any significant inhibition will resulting in a lower percentage number. FIG. 26B indicates that only one antibody significantly cross-reacted with the digested HAM peptides.

Also shown are two examples of synthesis of nearest neighbor peptides to the PET, which demonstrate that only the PET has good binding to the antibody (FIG. 20).

### Example 8

### Antibody Arrays

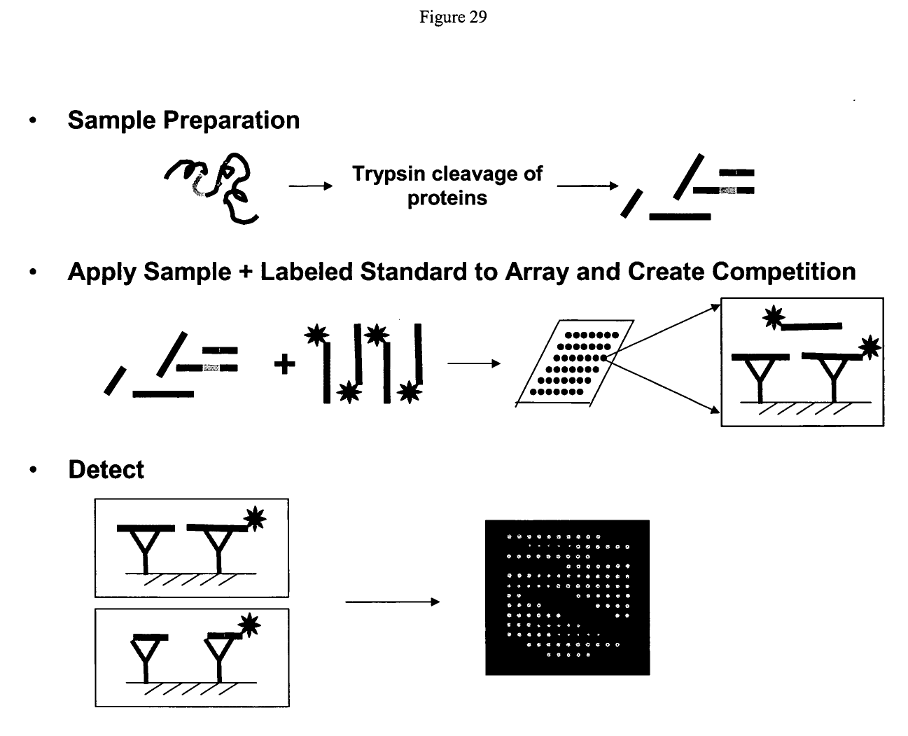

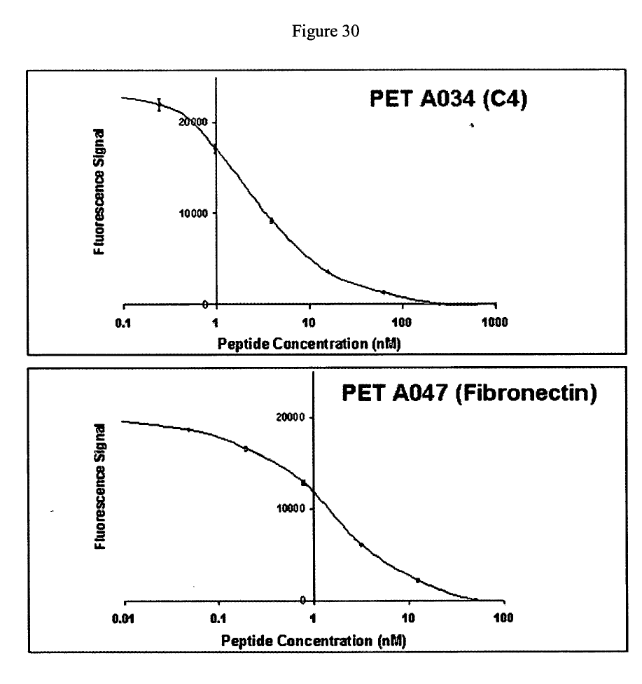

As described above, several formats of antibody arrays can be used in the instant invention. FIG. 29 shows a schematic drawing of a competitive antibody assay format with labeled peptide standards. An exemplary result of such an assay is shown in FIG. 30.

Capture antibody was printed on commercially available Poly-L Lysine slides (CEL Associates, Inc.) using a non-contact PiezzoArray printer (Perkin Elmer). A single 350 pL volume of antibody, at a concentration of 0.5 mg/ml was printed. The slide was blocked with a 6% BSA solution for 2 hours, and excess BSA was removed. The standard curve was prepared with a synthetic peptide standard varying in concentration from 0.1 to 1000 nM, incubated with a constant amount of a labeled version of the same synthetic peptide (Alex Fluor 555 Molecular Probes). At low peptide concentration, the antibody on the array binded exclusively to the labeled peptide and maximum assay signal was generated. As peptide concentrations increased, competition for antibody binding increased and less labeled peptide binded to the antibody on the array. The standard curve was prepared in a PBS buffer containing 6% BSA. Following a 1 hour incubation, the slide was washed five times in a PBS buffer containing 0.05% tween and then scanned in a fluorescent scanner (Scan Array HT, Perkin Elmer). The images were analyzed using the QuantArray software and the data was reduced on an Excel Spreadsheet.

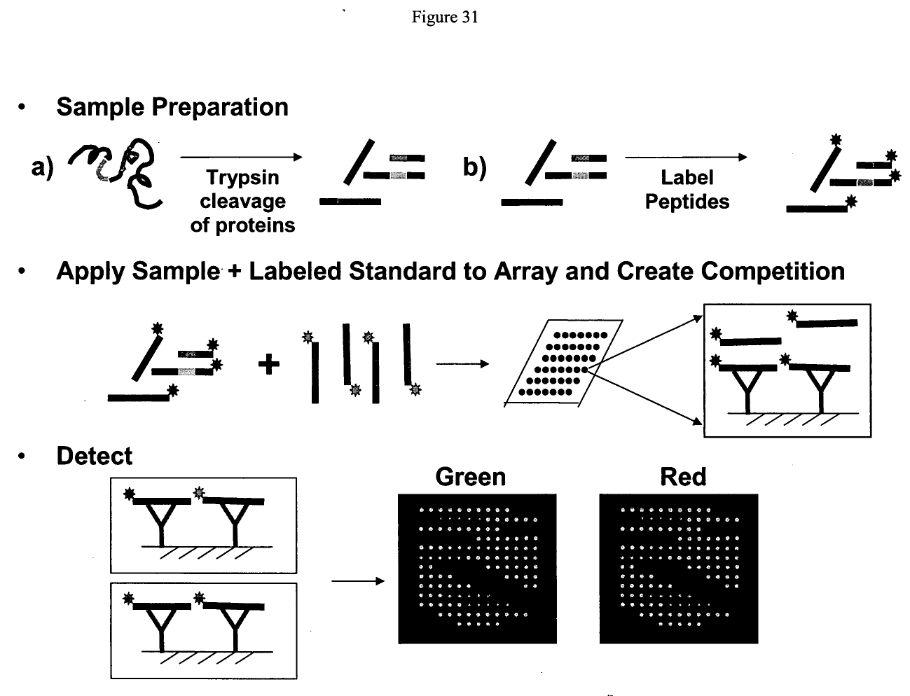

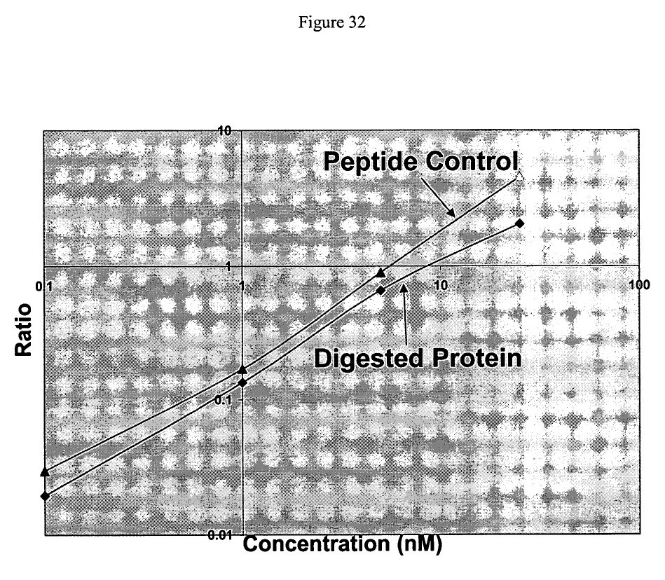

FIG. 31 shows a schematic drawing of a competitive antibody assay ratio format, with labeled peptide standards and labeled peptide fragments. An exemplary result of such an assay for TGF-β 1 is shown in FIG. 32.

In this example, a standard synthetic peptide was labeled with Alexa Fluor 555 (AF555) on the sulfhydryl of the cysteine residue and purified by HPLC. Another control peptide was labeled with Alexa Fluor 647 (AF647). Holding the AF555-labeled peptide concentration constant at 1 nM, the AF647-labeled peptide was spiked in at a range of concentrations. The various mixtures were then applied to a slide, onto which an antibody that recognizes the peptide was immobilized. After the antibody captured the labeled peptides, the slide was washed and scanned at both wavelengths to detect signals from AF555 and AF647 respectively. The ratio of the intensity of the two wavelengths was then plotted against the concentration of the AF647-labeled peptide to produce the red curve in FIG. 32. A similar experiment was then performed by digesting intact recombinant TGF-β1 with trypsin, and then labeling it with Alexa Fluor 647. It was then spiked in at various dilutions with a constant concentration of Alexa Fluor 555-labeled peptide and also incubated with the antibody on the slide, washed, and read at both wavelengths. The resultant curve was plotted above in blue.

### Example 9

### Sample Treatment

The example below relates to an efficient procedure for digesting serum samples. It appears to be greater than 95% efficient in terms of digesting intact protein, and requires only a 2-5-fold dilution for the entire process. The entire standard process takes about 18 hours due to an overnight tryptic digestion, but data indicates that as few as 5 minutes at 37° C. (if not less) are sufficient for almost complete digestion. Thus digestion process can be reduced to under 4 hours.

LC/MS analysis also indicates that we have near complete terminal digestion. Furthermore, alkylation of cysteines is also complete.

Also shown is a method for labeling the peptides with NHS activated Alexa Fluors. In a preferred embodiment, a molar excess of label over peptides is used. The procedure is adapted from Molecular Probes document MP00143 and Miller, J. C. et al. Proteomics 2003, 3:56-63 (incorporated herein by reference).

Reagents

NHS Alexa Fluor 555 or 647 (˜500 μg/sample)
DMSO (dry ˜200 μl)
1 M Tris, pH 8 (100 mL)
200 mM Sodium Bicarbonate, pH 8.3 (100 mL)
Serum digest sample (35 μl at 2 mg/ml)
   Procedure
   1. Digest serum at 1/10 dilution in carbonate buffer. (10 μl serum+90 μl sodium carbonate buffer). Final dilution is 1/35. Use 35 μl in this labeling.
   2. Prepare NHS Alexa Fluor reagent by dissolving at 10 mg/ml into dry DMSO. (e.g. 5 mg dye in 0.5 ml DMSO).
   3. Slowly add 45 ul NHS Alexa Fluor reagent to 35 μl serum digest sample by pipetting slowly while vortexing.
   4. Allow to react at room temperature for 1 hour.
   5. Quench by adding 50 μl 1 M Tris, pH 8.0 and allow to react at room temperature for 1 hour.

Although many proteases may be used for digestion, trypsin is preferred.

Prior to digestion, most proteins are preferably reduced and/or denatured. This involves unfolding of the protein and reduction of the disulfide cross-links between cysteine residues. To ensure that the cysteine residues do not re-link, they may be capped with an alkylating reagent (e.g. iodoacetic acid, iodoacetamide, 4-vinylpyridine, etc.). Although not necessarily in that order, reduction and denaturation are usually done in one step, followed by alkylation in a second step.

Materials:

Trypsin, TPCK treated bovine pancreatic (Sigma T-8802; Swiss Prot Acc: P00760);
TCEP, Tris(2-Carboxyethyl) Phosphine, neutralized (Pierce 77720);
Sodium dodecyl sulfate (SDS);
Iodoacetamide (Sigma I-1149);
200 mM NaHCO₃ pH 8.3 (this pH may drift upwards over time);
AlexaFluor 647 (Molecular Probes, A20106)

Trypsin may be advantageous since it is predictable in cleavage, and tryptic fragments are small enough that they are generally soluble in aqueous buffer systems. Preferably, TPCK treatment is used to reduce the chymotryptic activity that accompanies purified trypsin. Bovine trypsin is preferred in certain embodiments, since it has been sequenced and is recorded in the SwissProt database, allowing easy identification of autolytic fragments of this protein. Furthermore, trypsin autolytic fragments will not affect our PET assays provided that the trypsin has been inactivated or removed prior to analysis.

In preferred embodiments, TCEP was chosen for reduction because it is highly stable in a shippable liquid form, is quantitative in reduction, and does not directly compete for alkylating reagent. Alternatively, DTT and mercaptoethanol may be used in great excess, because the reduction is competitive. TCEP may also be used at acidic pH if necessary. Compared to other phosphine reagents, it is more easily shipped because it is not pyrophoric.

SDS was the preferred agent for denaturation. It can be used effectively at low concentration (0.05%), and does not need to be diluted or removed prior to tryptic digestion. It also does not appear to directly interfere with the PET assays at this concentration. If necessary, it can be removed by cation exchange chromatography of the peptide digest—it flows through while the tryptic fragments are retained.

Iodoacetamide was the preferred alkylating reagent. It is commonly used for alkylation, and can be shipped lyophilized or in solution. It does not add charge to the peptide being alkylated. Other potential choices include iodoacetic acid, 4-vinylpyridine, and N-ethylmaleimide, etc.

Reduction, alkylation, digestion and NHS labeling can all be carried out at slightly alkaline pH, around pH 8-9. NHS labeling requires a buffer that does not contain primary amines (e.g. tris(hydroxymethyl)aminoethane, Tris, can not be used).

In one embodiment, sodium bicarbonate buffer is used. It buffers at the required pH, and is non-reactive to the NHS chemistry. In addition, this buffer permits reduction and alkylation at high serum protein concentrations (ca. 15 mg/ml) without the precipitation observed with many other buffer systems (e.g. MOPS, Tricine, Tris). This buffer system works well, although the pH tends to drift over time if it is exposed to CO₂ in the air.

In another embodiment, triethanolamine buffer achieves all of the objectives mentioned previously. It is a tertiary amine so it is also non-reactive to NHS chemistry. When used at 50 mM, it can easily be neutralized after completion of digestion with a 1/10 volume of 500 mM dibasic phosphate to about pH 7.3, which is compatible with subsequent immunoassays. Either buffer system will work satisfactorily, but the triethanolamine has the advantage of increased pH stability.

Exemplary Digestion Protocol:

Materials:

200 mM NaHCO₃ pH 8.3
20×TCEP and SDS (100 mM TCEP, 1.0% SDS in water)
100 mM Iodoacetamide vial, prepared by adding 1 ml water to lyophilized reagent
6 mM HCl
1 mg lyophilized trypsin aliquot
   Procedure:
1. Dilute 100 μl serum with 300 μl NaHCO₃;
2. Add 20 μl 20×TCEP/SDS and heat for 5 minutes at 100° C.;
3. Cool to room temperature and add 40 μl 100 mM Iodoacetamide;
4. Incubate at room temperature in the dark for 30 minutes;
5. Add 100 μl 6 mM HCl to the 1 mg trypsin vial and resuspend;
6. Add 30 μl of the resuspended trypsin to the alkylated serum sample;
7. Digest overnight at 37° C.;
8. Inactivate trypsin by heating sample at 100° C. for 5 minutes.
   Experimental Results:

Completeness of Digestion

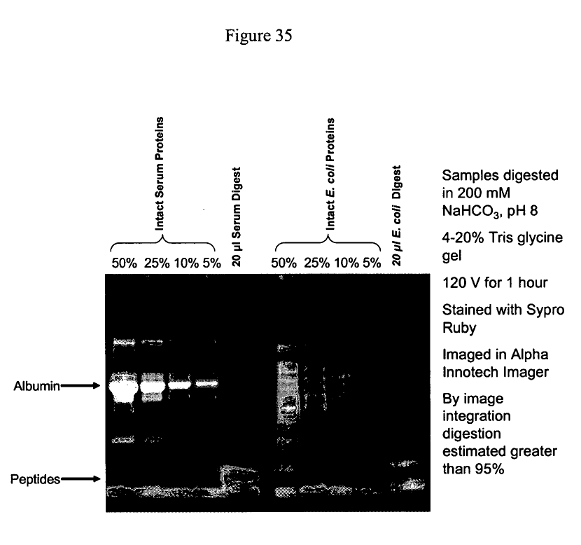

Serum was reduced, alkylated and digested using the protocol above. It was then analyzed on a SDS-PAGE gel against a dilution series of an intact serum sample. Even when 20 times more digested sample than intact sample dilution was loaded on the gel, the intensity of the remaining (undigested) proteins in the digested sample was less (FIG. 35). We therefore conclude that the digest was more than 95% efficient.

Completeness of digestion was also analyzed by LC/MS/MS. In this case, we examined tryptic fragments that were not terminal fragments (i.e., those containing internal lysines or arginines that were not adjacent to praline residues). The partial table below was generated from LC/MS/MS analysis on the LCQ for several human protein tryptic digests. Results were generated by Turbo Sequest in BioWorks.

| PET | Protein | Mass | Charge | Xcorr | ions |
|---|---|---|---|---|---|
| A021 | PSA | 1272.669 | 2 | 3.411 | 17/22 |
| A034 | C4 | 1984.004 | 3 | 3.754 | 32/64 |
| A036 | C9 | 3249.529 | 3 | 0.342 | 19/112 |
| A045 | HFBN | 3179.397 | 4 | 0.437 | 8/100 |

Specifically, recombinant proteins were reduced, alkylated, and digested with trypsin using the sample preparation protocol, and then analyzed by LC/MS/MS. Among the many peptides identified for each protein, the PET peptides above were also identified with varying degrees of confidence (Xcorr values >2 are very good). The sequences found show all of the cysteines with a “*” (results not shown), indicating complete alkylation. Only one missed cleavage was identified in A045 prior to the modified cysteine.

Alkylation of Cysteines

Completeness of alkylation was determined on the same C4 digest as above, but by setting up Turbo Sequest to make alkylation of cysteine a variable modification, so that each time an alkylated cysteine is found, it is designated with C*. Nearly all of the cysteines were starred (result not shown).

Minimization of Dilution

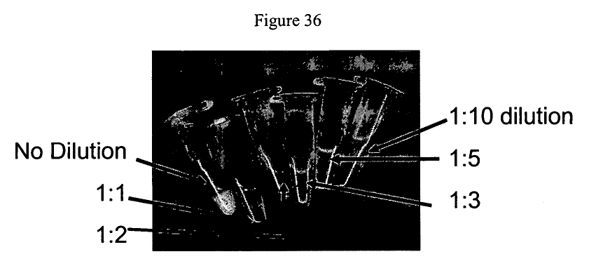

Determination of the minimum dilution was done by diluting human serum into varying amounts of 200 mM NaHCO₃ and performing the reduction at 100° C. for 5 min. FIG. 36 shows that at least a 1:2 dilution (i.e. 3 fold dilution) is required with this buffer system.

Decreased Digestion Time

Our current protocol calls for the standard overnight digestion at 37° C. To determine the amount of time required for complete digestion, a serum sample was first reduced and alkylated. Trypsin was added to this sample and aliquots removed and tryptic activity stopped by heating at 100° C. for 5 min. SDS-PAGE analysis indicates that the trypsin cleaves a significant amount of the protein in less than 5 minutes.

### Example 10

### Redundant Measurement

In this example, The concept of redundant protein measurement was demonstrated for the protein Fibronectin, where two different anti-PET antibodies directed at different portions of the protein sequence were shown to have similar competitive titration curves.

Synthetic peptides containing PET sequences representing 33 different array elements, including two PETs from the protein Fibronectin were printed on commercially available Aminopropyl Silane slides (Erie Scientific) using a non-contact PiezzoArray printer (Perkin Elmer). 350 pL volumes of each peptide, at a concentration of 25 μM were printed in several replicates. The slide was blocked with a 6% BSA solution for 2 hours, and excess BSA was then removed.

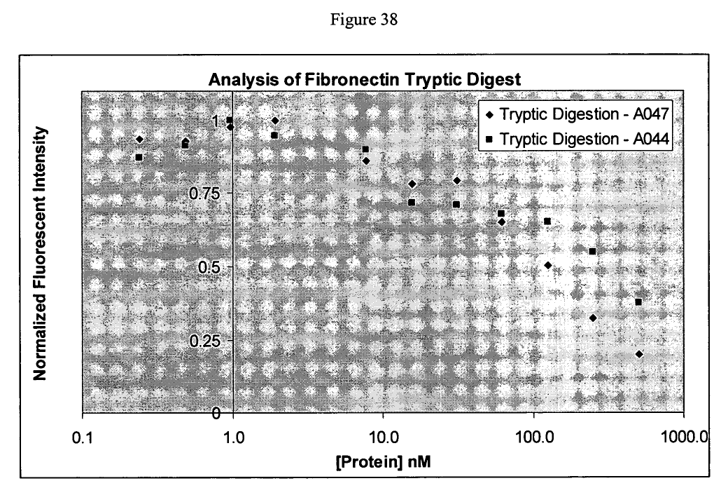

A 10 mg/ml solution of Human Fibronectin (HFBN-796, Molecular Innovations) was digested with trypsin. The tryptic digest, at total protein concentrations ranging from 0.1-1000 nM was titrated against a constant concentration of 4 nM antibody. At low tryptic digest concentration, the antibody binded exclusively to the peptide on the array, and maximum assay signal was generated. As digest concentration increased, competition for antibody binding increased and less antibody binded to the peptide on the array. The titration curves for the two different antibodies were prepared in a PBS buffer containing 6% BSA. Following 1 hour incubation, the slides were washed 5 times with a PBS buffer containing 0.05% tween. A goat-anti-rabbit detection antibody at a concentration of 2 nM, labeled with Alex Fluor 555 (Molecular Probes) was introduced. Following an 1 hour incubation, the slide was washed again and then scanned in a fluorescent scanner (Scan Array HT, Perkin Elmer). The images were analyzed using the QuantArray software and the data was reduced on an Excel Spreadsheet. A representative result was shown in FIG. 38.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

Equivalents

Those skilled in the art will recognize, or be able to ascertain using no more than routine experimentation, many equivalents to the specific embodiments of the invention described herein. Such equivalents are intended to be encompassed by the following claims.

1. A method for obtaining one or more capture agent(s) for identifying one or more target proteins in a sample, the method comprising:
(1) computationally identifying the amino acid sequences of one or more fragments of each said target proteins expected to be present in a variegated sample of proteins, said fragments predictably resulting from a treatment of said target proteins, and each of said fragments encompassing one or more unique PET (proteome epitope tag) sequences;
(2) generating reference reagents for each said unique PET sequences;
(3) obtaining a set of capture agents, each of which selectively binds a PET sequence of one of said reference reagents,
wherein collectively said set of capture agents can bind and identify the occurrence of said target proteins present in said sample under conditions wherein said capture agents are contacted with said target proteins, or said fragments thereof, that have been rendered soluble in solution.

2. The method of claim 1, wherein said step of computationally identifying amino acid sequences includes a Nearest-Neighbor Analysis that identifies PET sequences based on criteria that also include one or more of pI, charge, steric, solubility, hydrophobicity, polarity and solvent exposed area.

3. The method of claim 1, wherein said PET sequence is about 5-30 amino acids in length, preferably about 5-10 amino acids in length, most preferably about 8 amino acids.

4. The method of claim 1, wherein said capture agents are full-length antibodies, or functional antibody fragments selected from: Fab fragments, F(ab′)₂ fragments, Fd fragments, Fv fragments, dAb fragments, isolated complementarity determining regions (CDR), single chain antibodies (scFv), or derivatives thereof.

5. The method of claim 4, wherein at least about 50%, 60%, 70%, 80%, 90% or more of all of said antibodies or functional antibody fragments have affinity constants of no more than about 10 nM.

6. The method of claim 4, further comprising determining the specificity of said antibodies or functional antibody fragments against one or more nearest neighbor antigens, if any, of said PETs, and selecting antibodies or functional antibody fragments that do not substantially cross-react with any other antigens, including their nearest neighbor antigens.

7. The method of claim 1, further comprising derivatizing said capture agents with a detectable label.

8. The method of claim 1, wherein said reference reagents are natural or synthesized antigens comprising said PET sequence, and wherein the N- or C-terminus, or both, of said PET sequence are blocked to eliminate free N- or C-terminus, or both.

9. The method of claim 1, wherein step (3) is effectuated by screening libraries of antibodies or functional antibody fragments, or by de novo antibody production and screening using immunized animals.

10. A method for simultaneously detecting and/or measuring a plurality of target proteins in a sample, the method comprising:
(1) using the method of claim 1, obtaining a plurality of capture agents, each specific for one PET sequence of one of said target proteins, wherein each of said plurality of target proteins is recognized by at least one of said plurality of capture agents;
(2) treating the sample with a predetermined protocol to generate a plurality of poplypeptide fragments, wherein for each of said target proteins, at least one of its polypeptide fragments comprises at least one PET sequence recognized by at least one of said capture agents;
(3) contacting at least a portion of the treated sample with said plurality of capture agents, and,
(4) detecting/measuring binding events, thereby simultaneously detecting and/or measuring said plurality of target proteins in said sample.

11. The method of claim 10, wherein said predetermined protocol comprises denaturing and/or proteolysis of said sample.

12. The method of claim 1, wherein said sample is whole blood, plasma, serum, whole cell lysate, cell fraction obtained by lysis and fractionation of cellular material, extract or fraction of cells obtained directly from a biological entity, or cells grown in an artificial environment.

13. The method of claim 11, wherein said proteolysis is effected by a protease selected from: trypsin, chymotrypsin, pepsin, papain, carboxypeptidase, calpain, subtilisin, gluc-C, endo lys-C or proteinase K.

14. The method of claim 11, wherein said denaturing is effected by thermo-denaturation or chemical denaturation.

15. The method of claim 14, wherein said thermo-denaturation is followed by or concurrent with proteolysis using thermo-stable proteases.

16. The method of claim 10, wherein each of said capture agents is immobilized on a solid support at an addressable location; and wherein for each of said target proteins, each of said at least one of its polypeptide fragments further comprises a second PET sequence or a post-translational modification site.

17. The method of claim 16, wherein said post-translational modification site is a phosphorylation site for phospho-Tyr, Phospho-Ser, or phospho-Thr.

18. The method of claim 17, wherein said phosphorylation site is phosphorylated, and wherein step (4) is effectuated by detecting/measuring said second PET and/or phospho-amino acid at said phosphorylation site by a detectable agent (e.g. labeled secondary antibody or a fluorescent dye).

19. The method of claim 10, wherein each of said capture agents is immobilized on a solid support at an addressable location; and wherein step (3) further comprises simultaneously contacting said capture agents with labeled standard competition peptides, said labeled standard competition peptides are detected/measured in (4).

20. The method of claim 10, wherein each of said capture agents is immobilized on a solid support at an addressable location; wherein step (2) further comprises labeling said plurality of polypeptide fragments with a first label; wherein step (3) further comprises simultaneously contacting said capture agents with standard competition peptides labeled by a second label, said first and second labels are detected/measured in (4).

21. The method of claim 10, wherein each of said capture agents is immobilized on a solid support at an addressable location; and wherein at least one of said target proteins is represented by at least two polypeptide fragments, each comprising at least one PET sequence recognized by at least one of said capture agents.

22. The method of claim 10, further comprising generating an array of reference peptide fragments, each immobilized on an addressable location on a solid support, wherein each of said reference peptide fragments corresponds to one of said at least one polypeptide fragments; and wherein step (3) is carried out on said array.

23. The method of claim 22, wherein step (4) is effectuated by detecting/measuring said capture agents bound to said arrays.


SEQUENCE LISTING (232 sequences)

SEQ ID NO: 1 - protein, 11 aa, Unknown Organism
  Peptide used to illustrate phosphorylation site
YLRRASLAQLT

SEQ ID NO: 2 - protein, 8 aa, Unknown Organism
  Peptide used to illustrate phosphorylation site
FRRLSIST

SEQ ID NO: 3 - protein, 11 aa, Unknown Organism
  Peptide used to illustrate phosphorylation site
AGARRKASGPP

SEQ ID NO: 4 - protein, 11 aa, Unknown Organism
  Peptide used to illustrate phosphorylation site
RTLSVSSLPGL

SEQ ID NO: 5 - protein, 10 aa, Unknown Organism
  Peptide used to illustrate phosphorylation site
DIGSESTEDQ

SEQ ID NO: 6 - protein, 10 aa, Unknown Organism
  Peptide used to illustrate phosphorylation site
ADSESEDEED

SEQ ID NO: 7 - protein, 11 aa, Unknown Organism
  Peptide used to illustrate phosphorylation site
LESEEEGVPST

SEQ ID NO: 8 - protein, 11 aa, Unknown Organism
  Peptide used to illustrate phosphorylation site
EDNSEDEISNL

SEQ ID NO: 9 - protein, 9 aa, Unknown Organism
  Peptide used to illustrate phosphorylation site
SVPPSPSLS

SEQ ID NO: 10 - protein, 9 aa, Unknown Organism
  Peptide used to illustrate phosphorylation site
SVPPSPSLS

SEQ ID NO: 11 - protein, 7 aa, Unknown Organism
  Peptide used to illustrate phosphorylation site
PAKTPVK

SEQ ID NO: 12 - protein, 10 aa, Unknown Organism
  Peptide used to illustrate phosphorylation site
HSTPPKKKRK

SEQ ID NO: 13 - protein, 11 aa, Unknown Organism
  Peptide used to illustrate phosphorylation site
NYLRRRLSDSN

SEQ ID NO: 14 - protein, 10 aa, Unknown Organism
  Peptide used to illustrate phosphorylation site
KMARVFSVLR

SEQ ID NO: 15 - protein, 4 aa, Unknown Organism
  Peptide used to illustrate phosphorylation site
PLSP

SEQ ID NO: 16 - protein, 4 aa, Unknown Organism
  Peptide used to illustrate phosphorylation site
PSSP

SEQ ID NO: 17 - protein, 4 aa, Unknown Organism
  Peptide used to illustrate phosphorylation site
VLSP

SEQ ID NO: 18 - protein, 11 aa, Unknown Organism
  Peptide used to illustrate phosphorylation site
GKKRKRSRKES

SEQ ID NO: 19 - protein, 8 aa, Unknown Organism
  Peptide used to illustrate phosphorylation site
FRRLSIST

SEQ ID NO: 20 - protein, 12 aa, Unknown Organism
  Peptide used to illustrate phosphorylation site
DQEKRKQISVRG

SEQ ID NO: 21 - protein, 10 aa, Unknown Organism
  Peptide used to illustrate phosphorylation site
PLSRTLSVSS

SEQ ID NO: 22 - protein, 9 aa, Unknown Organism
  Peptide used to illustrate phosphorylation site
HEGTHSTKR

SEQ ID NO: 23 - protein, 10 aa, Unknown Organism
  Peptide used to illustrate phosphorylation site
PLSRTLSVSS

SEQ ID NO: 24 - protein, 11 aa, Unknown Organism
  Peptide used to illustrate phosphorylation site
QKRPSQRSKYL

SEQ ID NO: 25 - protein, 11 aa, Unknown Organism
  Peptide used to illustrate phosphorylation site
RENAEYLRVAP

SEQ ID NO: 26 - protein, 10 aa, Unknown Organism
  Peptide used to illustrate phosphorylation site
AEPDYGALYE

SEQ ID NO: 27 - protein, 8 aa, Homo sapiens
YVLEGVPH

SEQ ID NO: 28 - protein, 14 aa, Unknown Organism
  Illustrative peptide having high affinity for its antigen
GATPEDLNQKLAGN

SEQ ID NO: 29 - protein, 17 aa, Tobacco mosaic virus
CRGTGSYNRSSFESSSG

SEQ ID NO: 30 - protein, 15 aa, Unknown Organism
  Illustrative peptide having high affinity for its antigen
NYRAYATEPHAKKKS

SEQ ID NO: 31 - protein, 10 aa, Homo sapiens
RYDIEAKVTK

SEQ ID NO: 32 - protein, 8 aa, Unknown Organism
  Illustrative peptide having high affinity for its antigen
DRVYIHPF

SEQ ID NO: 33 - protein, 12 aa, Homo sapiens
PQSDPSVEPPLS

SEQ ID NO: 34 - protein, 8 aa, Artificial Sequence
  Synthetic HA tag
YDVPDYAS

SEQ ID NO: 35 - protein, 8 aa, Artificial Sequence
  Synthetic FLAG tag
MDYKAFDN

SEQ ID NO: 36 - protein, 5 aa, Artificial Sequence
  Synthetic 5-His tag
HHHHH

SEQ ID NO: 37 - protein, 8 aa, Homo sapiens
EPAELTDA

SEQ ID NO: 38 - protein, 8 aa, Homo sapiens
YEVQGEVF

SEQ ID NO: 39 - protein, 8 aa, Homo sapiens
GYSIFSYA

SEQ ID NO: 40 - protein, 12 aa, Homo sapiens
LSEPAELTDAVK

SEQ ID NO: 41 - protein, 16 aa, Homo sapiens
DEPVELTSAPTGHTFS

SEQ ID NO: 42 - protein, 17 aa, Homo sapiens
AGEAAELQDAEVESSAK

SEQ ID NO: 43 - protein, 15 aa, Homo sapiens
LQEPAELVESDGVPK

SEQ ID NO: 44 - protein, 12 aa, Homo sapiens
AQPAELVDSSGW

SEQ ID NO: 45 - protein, 14 aa, Homo sapiens
GLDPTQLTDALTQR

SEQ ID NO: 46 - protein, 10 aa, Homo sapiens
YEVQGEVFTK

SEQ ID NO: 47 - protein, 11 aa, Homo sapiens
HVEVNGEVFQK

SEQ ID NO: 48 - protein, 11 aa, Homo sapiens
SYEVLGEEFDR

SEQ ID NO: 49 - protein, 13 aa, Homo sapiens
QYAVSGEIFVVDR

SEQ ID NO: 50 - protein, 11 aa, Homo sapiens
VYEEQGEIILK

SEQ ID NO: 51 - protein, 11 aa, Homo sapiens
LYEVRGETYLK

SEQ ID NO: 52 - protein, 8 aa, Homo sapiens
EPVELTSA

SEQ ID NO: 53 - protein, 8 aa, Homo sapiens
DPTQLTDA

SEQ ID NO: 54 - protein, 8 aa, Homo sapiens
VEVNGEVF

SEQ ID NO: 55 - protein, 8 aa, Homo sapiens
YEVLGEEF

SEQ ID NO: 56 - protein, 24 aa, Homo sapiens
HSQPWQVAVYSHGWAHCGGVLVHR

SEQ ID NO: 57 - protein, 33 aa, Homo sapiens
IVGGWECEQHSQPWQAALYHFSTFQCGGILVHK

SEQ ID NO: 58 - protein, 24 aa, Homo sapiens
GSQPWQVSLFNGLSFHCAGVLVDR

SEQ ID NO: 59 - protein, 16 aa, Homo sapiens
NSQPWQVGLFEGTSLR

SEQ ID NO: 60 - protein, 29 aa, Homo sapiens
HECQPHSQPWQAALFQGQQLLCGGVLVGR

SEQ ID NO: 61 - protein, 29 aa, Homo sapiens
EDCSPHSQPWQAALVMENELFCSGVLVHR

SEQ ID NO: 62 - protein, 33 aa, Homo sapiens
VLNTNGTSGFLPGGYTCFPHSQPWQAALLVQGR

SEQ ID NO: 63 - protein, 21 aa, Homo sapiens
LLEGDECAPHSQPWQVALYER

SEQ ID NO: 64 - protein, 15 aa, Homo sapiens
PNSQPWQAGLFHLTR

SEQ ID NO: 65 - protein, 22 aa, Homo sapiens
CVTAGTSCLISGWGSTSSPQLR

SEQ ID NO: 66 - protein, 32 aa, Homo sapiens
VMDLPTQEPALGTTCYASGWGSIEPEEFLTPK

SEQ ID NO: 67 - protein, 44 aa, Homo sapiens
RLNPQVRPAVLPTRCPHPGEACVVSGWGLVSHEPGTAGSPRSQG

SEQ ID NO: 68 - protein, 5 aa, Homo sapiens
RLNPQ

SEQ ID NO: 69 - protein, 40 aa, Homo sapiens
RLNPQGDSGGPLVCGGILQGIVSWGDVPCDNTTKPGVYTK

SEQ ID NO: 70 - protein, 8 aa, Homo sapiens
SGWGLVSH

SEQ ID NO: 71 - protein, 8 aa, Homo sapiens
AGWGIVNH

SEQ ID NO: 72 - protein, 8 aa, Homo sapiens
SGWGITNH

SEQ ID NO: 73 - protein, 8 aa, Homo sapiens
SGWGMVTE

SEQ ID NO: 74 - protein, 8 aa, Homo sapiens
WGDVPCDN

SEQ ID NO: 75 - protein, 8 aa, Homo sapiens
WKDVPCED

SEQ ID NO: 76 - protein, 8 aa, Homo sapiens
WNDAPCDS

SEQ ID NO: 77 - protein, 8 aa, Homo sapiens
WNDAPCDK

SEQ ID NO: 78 - protein, 19 aa, Artificial Sequence
  Description of Artificial Sequence Synthetic peptide tag
MDYKAFDNLEQKLISEEDL

SEQ ID NO: 79 - protein, 28 aa, Homo sapiens
  MOD_RES (8): phosphoTyr
NSFNNPAYYVLEGVPHQLLPPEPPSPAR

SEQ ID NO: 80 - protein, 8 aa, Homo sapiens
YLTEGVPH

SEQ ID NO: 81 - protein, 8 aa, Homo sapiens
YILESMPH

SEQ ID NO: 82 - protein, 8 aa, Homo sapiens
YVIMGMPH

SEQ ID NO: 83 - protein, 15 aa, Homo sapiens
  MOD_RES (6): phosphoTyr
LGGGQYGEVYEGVWK

SEQ ID NO: 84 - protein, 8 aa, Homo sapiens
EVYVGVWK

SEQ ID NO: 85 - protein, 8 aa, Homo sapiens
EVFEGLWK

SEQ ID NO: 86 - protein, 8 aa, Homo sapiens
EVYEGVYT

SEQ ID NO: 87 - protein, 23 aa, Unknown Organism
  Description of Unknown Organism  Illustrative parental tryptic peptide
WSGSHQFEQLSGSILWMAPEVIR

SEQ ID NO: 88 - protein, 16 aa, Unknown Organism
  Description of Unknown Organism  Illustrative parental tryptic peptide
MSFAGTVAWMAPEVIR

SEQ ID NO: 89 - protein, 18 aa, Unknown Organism
  Description of Unknown Organism  Illustrative parental tryptic peptide
SFIGTPYWMAPEVAAVER

SEQ ID NO: 90 - protein, 12 aa, Unknown Organism
  Description of Unknown Organism  Illustrative parental tryptic peptide
WMAPEVFTQCTR

SEQ ID NO: 91 - protein, 20 aa, Unknown Organism
  Description of Unknown Organism  Illustrative parental tryptic peptide
LSFIGTPYWMAPEVAAVALK

SEQ ID NO: 92 - protein, 21 aa, Unknown Organism
  Description of Unknown Organism  Illustrative parental tryptic peptide
DSFIGTPYWMAPEVVMCETMK

SEQ ID NO: 93 - protein, 16 aa, Unknown Organism
  Description of Unknown Organism  Illustrative parental tryptic peptide
MSFAGTVAWMAPEVIR

SEQ ID NO: 94 - protein, 21 aa, Unknown Organism
  Description of Unknown Organism  Illustrative parental tryptic peptide
SMHGTPYWMAPEVINESGYGR

SEQ ID NO: 95 - protein, 24 aa, Unknown Organism
  Description of Unknown Organism  Illustrative parental tryptic peptide
NTVIGTPFWMAPEVIQEIGYNCVR

SEQ ID NO: 96 - protein, 23 aa, Unknown Organism
  Description of Unknown Organism  Illustrative parental tryptic peptide
NTFVGTPFWMAPEVIQQSAYDSK

SEQ ID NO: 97 - protein, 28 aa, Unknown Organism
  Description of Unknown Organism  Illustrative parental tryptic peptide
NTSVGTPFWMAPEVIACEQQLDTTYDAR

SEQ ID NO: 98 - protein, 28 aa, Unknown Organism
  Description of Unknown Organism  Illustrative parental tryptic peptide
NTSVGTPFWMAPEVIACEQQYDSSYDAR

SEQ ID NO: 99 - protein, 28 aa, Unknown Organism
  Description of Unknown Organism  Illustrative parental tryptic peptide
NTFIGTPYWMAPEVIACDENPDATYDYR

SEQ ID NO: 100 - protein, 18 aa, Unknown Organism
  Description of Unknown Organism  Illustrative parental tryptic peptide
TFVGTPCWMAPEVMEQVR

SEQ ID NO: 101 - protein, 17 aa, Unknown Organism
  Description of Unknown Organism  Illustrative parental tryptic peptide
STMVGTPYWMAPEVVTR

SEQ ID NO: 102 - protein, 16 aa, Unknown Organism
  Description of Unknown Organism  Illustrative parental tryptic peptide
SLVGTPYWMAPEVISR

SEQ ID NO: 103 - protein, 23 aa, Unknown Organism
  Description of Unknown Organism  Illustrative parental tryptic peptide
WSGSQQVEQPTGSVLWMAPEVIR

SEQ ID NO: 104 - protein, 18 aa, Unknown Organism
  Description of Unknown Organism  Illustrative parental tryptic peptide
TFVGTPCWMAPEVMEQVR

SEQ ID NO: 105 - protein, 33 aa, Unknown Organism
  Description of Unknown Organism  Illustrative parental tryptic peptide
ASMASPANSFVGTPYWMAPEVILAMDEGQYDGK

SEQ ID NO: 106 - protein, 33 aa, Unknown Organism
  Description of Unknown Organism  Illustrative parental tryptic peptide
ASIMAPANSFVGTPYWMAPEVILAMDEGQYDGK

SEQ ID NO: 107 - protein, 18 aa, Unknown Organism
  Description of Unknown Organism  Illustrative parental tryptic peptide
EPLAVVGSPYWMAPEVLR

SEQ ID NO: 108 - protein, 22 aa, Unknown Organism
  Description of Unknown Organism  Illustrative parental tryptic peptide
TTHMSLVGTFPWMAPEVIQSLR

SEQ ID NO: 109 - protein, 8 aa, Unknown Organism
  Description of Unknown Organism Coronavirus peptide
YSSLNDEF

SEQ ID NO: 110 - protein, 36 aa, Homo sapiens
KVLALYNQHNPGASAAPCCVPQALEPLPIVYYVGRK

SEQ ID NO: 111 - protein, 8 aa, Homo sapiens
NHHSPGGS

SEQ ID NO: 112 - protein, 8 aa, Homo sapiens
QQHNPAAN

SEQ ID NO: 113 - protein, 8 aa, Homo sapiens
NKHGPGVS

SEQ ID NO: 114 - protein, 8 aa, Homo sapiens
EPLTILYY

SEQ ID NO: 115 - protein, 8 aa, Homo sapiens
DPLPVRYY

SEQ ID NO: 116 - protein, 8 aa, Homo sapiens
EPLPSQYY

SEQ ID NO: 117 - protein, 36 aa, Homo sapiens
RVLSLYNTINPEASASPCCVSQDLEPLTILYYIGKT

SEQ ID NO: 118 - protein, 8 aa, Homo sapiens
NTLNPEAS

SEQ ID NO: 119 - protein, 8 aa, Homo sapiens
NKLDPEAS

SEQ ID NO: 120 - protein, 8 aa, Homo sapiens
NTANPERS

SEQ ID NO: 121 - protein, 8 aa, Homo sapiens
PQDLEPLT

SEQ ID NO: 122 - protein, 8 aa, Homo sapiens
SEDLEPLA

SEQ ID NO: 123 - protein, 8 aa, Homo sapiens
SQDLDPMA

SEQ ID NO: 124 - protein, 44 aa, Homo sapiens
RSADTTHSTVLGLYNTLNPEASASPCCVPQDLEPLTILYYVGRT

SEQ ID NO: 125 - protein, 8 aa, Homo sapiens
SAHSTHST

SEQ ID NO: 126 - protein, 8 aa, Homo sapiens
SSDTTHAS

SEQ ID NO: 127 - protein, 8 aa, Homo sapiens
AAEATHST

SEQ ID NO: 128 - protein, 8 aa, Homo sapiens
NTINPEAS

SEQ ID NO: 129 - protein, 8 aa, Homo sapiens
NKLDPEAS

SEQ ID NO: 130 - protein, 8 aa, Homo sapiens
NTANPERS

SEQ ID NO: 131 - protein, 41 aa, Homo sapiens
MKWAKNWVLEPPGFLAYECVGTCQQPPEALAFNWPFLGPRQ

SEQ ID NO: 132 - protein, 8 aa, Homo sapiens
NWAVDPPG

SEQ ID NO: 133 - protein, 8 aa, Homo sapiens
HWVVSPPG

SEQ ID NO: 134 - protein, 8 aa, Homo sapiens
NWVRLPPG

SEQ ID NO: 135 - protein, 8 aa, Homo sapiens
QPPEAFGF

SEQ ID NO: 136 - protein, 8 aa, Homo sapiens
KPPEALAM

SEQ ID NO: 137 - protein, 8 aa, Homo sapiens
QPPEAKKF

SEQ ID NO: 138 - protein, 6 aa, Homo sapiens
RHRRAL

SEQ ID NO: 139 - protein, 6 aa, Homo sapiens
RKKRAL

SEQ ID NO: 140 - protein, 8 aa, Homo sapiens
EQYYDPSD

SEQ ID NO: 141 - protein, 8 aa, Homo sapiens
EQYYDPTD

SEQ ID NO: 142 - protein, 8 aa, Homo sapiens
PTPIQLNP

SEQ ID NO: 143 - protein, 8 aa, Homo sapiens
TNLEALQK

SEQ ID NO: 144 - protein, 8 aa, Homo sapiens
PTPIPLQP

SEQ ID NO: 145 - protein, 8 aa, Homo sapiens
TNLESLEK

SEQ ID NO: 146 - protein, 8 aa, Homo sapiens
PTPIQPKP

SEQ ID NO: 147 - protein, 8 aa, Homo sapiens
TNVKALQK

SEQ ID NO: 148 - protein, 8 aa, Homo sapiens
PTPVQTHP

SEQ ID NO: 149 - protein, 8 aa, Homo sapiens
ENLEALQR

SEQ ID NO: 150 - protein, 8 aa, Homo sapiens
PQQIQLNP

SEQ ID NO: 151 - protein, 8 aa, Homo sapiens
SKLEALQK

SEQ ID NO: 152 - protein, 8 aa, Homo sapiens
PTPIEFSP

SEQ ID NO: 153 - protein, 8 aa, Homo sapiens
TNLEALEE

SEQ ID NO: 154 - protein, 14 aa, Homo sapiens
ITVEEALAHPYLEQ

SEQ ID NO: 155 - protein, 15 aa, Homo sapiens
DEPVAEEPFTFAMEL

SEQ ID NO: 156 - protein, 10 aa, Homo sapiens
IADPEHDHTG

SEQ ID NO: 157 - protein, 14 aa, Homo sapiens
IEVEQALAHPYLEQ

SEQ ID NO: 158 - protein, 13 aa, Homo sapiens
DPSDEPIAEAPFK

SEQ ID NO: 159 - protein, 10 aa, Homo sapiens
VADPDHDHTG

SEQ ID NO: 160 - protein, 12 aa, Homo sapiens
PTPIQLNPAPDG

SEQ ID NO: 161 - protein, 14 aa, Homo sapiens
GTSSAETNLEALQK

SEQ ID NO: 162 - protein, 9 aa, Homo sapiens
CDFGVSGQL

SEQ ID NO: 163 - protein, 15 aa, Homo sapiens
ALYNQHNPGASAAPC

SEQ ID NO: 164 - protein, 16 aa, Homo sapiens
PQALEPLPIVYYVGRC

SEQ ID NO: 165 - protein, 12 aa, Homo sapiens
CYIWSLDTQYSK

SEQ ID NO: 166 - protein, 15 aa, Homo sapiens
ITVEEALAHPYLEQC

SEQ ID NO: 167 - protein, 15 aa, Homo sapiens
CVAEEPFTFDMELDD

SEQ ID NO: 168 - protein, 11 aa, Homo sapiens
IADPEHDHTGC

SEQ ID NO: 169 - protein, 15 aa, Homo sapiens
IEVEQALAHPYLEQC

SEQ ID NO: 170 - protein, 14 aa, Homo sapiens
CDPSDEPIAEAPFK

SEQ ID NO: 171 - protein, 11 aa, Homo sapiens
VADPDHDHTGC

SEQ ID NO: 172 - protein, 13 aa, Homo sapiens
PTPIQLNPAPDGC

SEQ ID NO: 173 - protein, 15 aa, Homo sapiens
CGTSSAETNLEALQK

SEQ ID NO: 174 - protein, 13 aa, Homo sapiens
GGQSAESEEEELC

SEQ ID NO: 175 - protein, 11 aa, Homo sapiens
ELAETGLELLC

SEQ ID NO: 176 - protein, 12 aa, Homo sapiens
CEIYDTIFAGLD

SEQ ID NO: 177 - protein, 13 aa, Homo sapiens
CDFSTPLAHFDLR

SEQ ID NO: 178 - protein, 12 aa, Homo sapiens
CDEAGQELEGMR

SEQ ID NO: 179 - protein, 10 aa, Homo sapiens
AYATEPHAKC

SEQ ID NO: 180 - protein, 12 aa, Homo sapiens
NIDALSGMEGRC

SEQ ID NO: 181 - protein, 13 aa, Homo sapiens
LSEPAELTDAVKC

SEQ ID NO: 182 - protein, 11 aa, Homo sapiens
CDLPTQEPALG

SEQ ID NO: 183 - protein, 13 aa, Homo sapiens
CGSIEPEEFLTPK

SEQ ID NO: 184 - protein, 14 aa, Homo sapiens
CNEDDLFFEADGPK

SEQ ID NO: 185 - protein, 12 aa, Homo sapiens
SLVMSGPYELKC

SEQ ID NO: 186 - protein, 12 aa, Homo sapiens
PTLQLESVDPKC

SEQ ID NO: 187 - protein, 13 aa, Homo sapiens
CLEFESAQFPNWY

SEQ ID NO: 188 - protein, 12 aa, Homo sapiens
CENMPVFLGGTK

SEQ ID NO: 189 - protein, 12 aa, Homo sapiens
CHYLDETEQWEK

SEQ ID NO: 190 - protein, 13 aa, Homo sapiens
CQTDQPIYNPGQR

SEQ ID NO: 191 - protein, 14 aa, Homo sapiens
CEANEDYEDYEYDE

SEQ ID NO: 192 - protein, 12 aa, Homo sapiens
TPFDNEFYNGLC

SEQ ID NO: 193 - protein, 13 aa, Homo sapiens
CTEHYEEQIEAFK

SEQ ID NO: 194 - protein, 13 aa, Homo sapiens
PWNVASLIYETKC

SEQ ID NO: 195 - protein, 11 aa, Homo sapiens
CYEVQGEVFTK

SEQ ID NO: 196 - protein, 9 aa, Homo sapiens
CHEGGQSYK

SEQ ID NO: 197 - protein, 12 aa, Homo sapiens
CTFYQIGDSWEK

SEQ ID NO: 198 - protein, 11 aa, Homo sapiens
CTTQNYDADQK

SEQ ID NO: 199 - protein, 16 aa, Homo sapiens
VDVIPVNLPGEHGQRC

SEQ ID NO: 200 - protein, 15 aa, Homo sapiens
CVAGWGHQFEGAEEY

SEQ ID NO: 201 - protein, 30 aa, Homo sapiens
PTPIQLNPAPDGSAVNGTSSAETNLEALQK

SEQ ID NO: 202 - protein, 22 aa, Homo sapiens
LCDFGVSGQLIDSMANSFVGTR

SEQ ID NO: 203 - protein, 33 aa, Homo sapiens
PVLPALTINPTIAEGPSPTSEGASEAHLVDLQK

SEQ ID NO: 204 - protein, 19 aa, Homo sapiens
VADPDHDHTGFLTEYVATR

SEQ ID NO: 205 - protein, 29 aa, Homo sapiens
IEVEQALAHPYLEQYYDPSDEPIAEAPFK

SEQ ID NO: 206 - protein, 19 aa, Homo sapiens
IADPEHDHTGFLTEYVATR

SEQ ID NO: 207 - protein, 39 aa, Homo sapiens
ITVEEALAHPYLEQYYDPTDEPVAEEPFTFAMELDDLPK

SEQ ID NO: 208 - protein, 14 aa, Homo sapiens
IEVEQALAHPYLEQ

SEQ ID NO: 209 - protein, 14 aa, Homo sapiens
ISAKDALAHPYLDE

SEQ ID NO: 210 - protein, 14 aa, Homo sapiens
LTAEQALQHPFFER

SEQ ID NO: 211 - protein, 14 aa, Homo sapiens
YTAEEALAHPFFQQ

SEQ ID NO: 212 - protein, 14 aa, Homo sapiens
IAAHQALQHPYFQE

SEQ ID NO: 213 - protein, 14 aa, Homo sapiens
ITAAQALAHAYFAQ

SEQ ID NO: 214 - protein, 14 aa, Homo sapiens
ISVDEALQHPYINV

SEQ ID NO: 215 - protein, 14 aa, Homo sapiens
VTAGEALAHPYFES

SEQ ID NO: 216 - protein, 14 aa, Homo sapiens
ISAEEALQHPYFSD

SEQ ID NO: 217 - protein, 14 aa, Homo sapiens
LTCEQLLHHPYFEN

SEQ ID NO: 218 - protein, 13 aa, Homo sapiens
DPSDEPIAEAPFK

SEQ ID NO: 219 - protein, 13 aa, Homo sapiens
DPTDEPVAEEPFT

SEQ ID NO: 220 - protein, 13 aa, Homo sapiens
FPTDEPISSHPFR

SEQ ID NO: 221 - protein, 13 aa, Homo sapiens
DPSLPPLAQSPPK

SEQ ID NO: 222 - protein, 13 aa, Homo sapiens
KPTHKPIAEAPSA

SEQ ID NO: 223 - protein, 13 aa, Homo sapiens
DPDDEPDCAPPFD

SEQ ID NO: 224 - protein, 13 aa, Homo sapiens
DPSDEEAASSPKE

SEQ ID NO: 225 - protein, 13 aa, Homo sapiens
FPMDEPISSHPFH

SEQ ID NO: 226 - protein, 13 aa, Homo sapiens
DPSDLPVDRAATK

SEQ ID NO: 227 - protein, 13 aa, Homo sapiens
EPSFEPQAPAPYL

SEQ ID NO: 228 - protein, 13 aa, Homo sapiens
DKNKEPNAEEKFK

SEQ ID NO: 229 - protein, 30 aa, Homo sapiens; identical to SEQ ID NO: 201

SEQ ID NO: 230 - protein, 32 aa, Homo sapiens
KPTPIQLNPAPDGSAVNGTSSAETNLEALQKK

SEQ ID NO: 231 - protein, 22 aa, Homo sapiens
  MOD_RES 13, 17: phosphoSer
LCDFGVSGQLIDSMANSFVGTR

SEQ ID NO: 232 - protein, 26 aa, Homo sapiens
  MOD_RES 17, 21: phosphoSer
GEIKLCDFGVSGQLIDSMANSFVGTR